SEQUENCE LISTING (13 sequences)

SEQ ID NO: 1 - DNA, 20 bp, Artificial Sequence
  synthetic probe
gttggtgtgccactagttcc

SEQ ID NO: 2 - DNA, 20 bp, Artificial Sequence
  synthetic probe
ggaactagtggcacaccaac

SEQ ID NO: 3 - DNA, 27 bp, Artificial Sequence
  synthetic primer
gagatattattttcaatgggatagaac

SEQ ID NO: 4 - DNA, 23 bp, Artificial Sequence
  synthetic primer
caatgctctatttgtttgccatg

SEQ ID NO: 5 - DNA, 48 bp, Artificial Sequence
  synthetic primer
gaacatcatctggatctgtaccaaccatctcatactggaactagtggc

SEQ ID NO: 6 - DNA, 48 bp, Artificial Sequence
  synthetic primer
ctgtgccagtacacttactaagagtgttagtctacatggtttacaatc

SEQ ID NO: 7 - DNA, 23 bp, Artificial Sequence
  synthetic primer
tggtatatatttgttggtgtgcc

SEQ ID NO: 8 - DNA, 23 bp, Artificial Sequence
  synthetic primer
acaggtgatgaatttgctacagg

SEQ ID NO: 9 - DNA, 1000 bp, Parvo virus
aaacgctaatacgactcactatagggcgatctacgggtactttcaataatcagacggaatttaaatttttggaaaacggatgggtggaaatcacagcaaactcaagcagacttgtacatttaaatatgccagaaagtgaaaattatagaagagtggttgtaaataatttggataaaactgcagttaacggaaacatggctttagatgatactcatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttctaaattctttgcctcaagctgaaggaggtactaactttggttatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacccttccctttagtgagggttaataa

SEQ ID NO: 10 - DNA, 27 bp, Artificial Sequence
  synthetic probe
gaaggcatcctaagaaatcgctactac

SEQ ID NO: 11 - DNA, 28 bp, Artificial Sequence
  synthetic probe
tgctcggctgtatcatgaaacaaaagga

SEQ ID NO: 12 - DNA, 51 bp, Artificial Sequence
  synthetic probe
gtagtagcgatttcttaggatgccttctggtatatatttgttggtgtgcca

SEQ ID NO: 13 - DNA, 57 bp, Artificial Sequence
  synthetic probe
gtagtagcgatttcttaggatgccttctggtatatatttgttggtgtgccactagtt

(19) United States
(12) Patent Application Publication
(10) Pub. No.: US 2017/0191122 A1
(43) Pub. Date: Jul. 6, 2017

(54) METHOD OF DETECTING TARGET NUCLEIC ACID, ASSAY KIT AND NUCLEIC ACID PROBE IMMOBILIZED SUBSTRATE

(71) Applicant: KABUSHIKI KAISHA TOSHIBA, Minato-ku (JP)

(72) Inventors: Koji HASHIMOTO, Atsugi (JP); Naoko Nakamura, Machida (JP); Keiko Ito, Kawasaki (JP)

(73) Assignee: KABUSHIKI KAISHA TOSHIBA, Minato-ku (JP)

(21) Appl. No.: 15/420,917

(22) Filed: Jan. 31, 2017

Related U.S. Application Data
(63) Continuation of application No. PCT/JP2015/080957, filed on Nov. 2, 2015.

(30) Foreign Application Priority Data
Feb. 27, 2015 (JP) 2015-038670

(51) Int. Cl. C12Q 1/68

(52) U.S. Cl. CPC C12Q 1/6837; C12Q 1/6851; C12Q 1/6823; C12Q 1/6825

(57) ABSTRACT

According to one embodiment, a method for detecting a target nucleic acid includes (A) placing a reaction field formed by a reaction solution under an isothermal amplification reaction condition, the reaction solution including a sample which includes the target nucleic acid, a nucleic acid probe, a covering nucleic acid chain, a labeling substance, and a primer set, (B) monitoring or detecting the signal under the isothermal amplification reaction condition, and (C) obtaining a detection result, and the detection of the detectable signal produced by the labeling substance is inhibited by a presence of the nucleic acid which is bonded to the nucleic acid probe.

### CROSS REFERENCE TO RELATED APPLICATIONS

This application is a Continuation Application of PCT Application No. PCT/JP2015/080957, filed Nov. 2, 2015 and based upon and claiming the benefit of priority from Japanese Patent Application No. 2015-038670, filed Feb. 27, 2015, the entire contents of all of which are incorporated herein by reference.

### FIELD

Embodiments described herein relate generally to a method of detecting target nucleic acid, an assay kit, and a nucleic acid probe immobilized substrate.

### BACKGROUND

As genetic test technology is developed, nucleic acid tests are conducted in various scenes including clinical diagnosis and criminal investigation. Quantification of the nucleic acid becomes important to efficiently conduct a subsequent test and analyze a gene expression amount.

Real-time PCR, the microarray method, etc. are known as methods of quantifying nucleic acids.

In real-time PCR, sensitivity is high due to amplification of a nucleic acid, and analysis can be conducted in a wide quantitative range. In contrast, if the type of nucleic acids to be detected is increased, analysis needs to be conducted for each of the types. On the other hand, more than several tens of thousands of types of nucleic acids can be simultaneously analyzed in the microarray method. However, sensitivity and accuracy in quantitative analysis are inferior to those of the real-time PCR.

### BRIEF DESCRIPTION OF THE DRAWINGS

FIG. 1 is flowcharts showing a method of detecting a nucleic acid of the embodiment.

FIG. 2 is schematic illustrations showing a nucleic acid probe immobilized substrate of one of embodiments.

FIG. 3 is schematic illustration showing the nucleic acid probe immobilized substrate of the embodiment in which a first sequence and a second sequence of a covering nucleic acid chain are sequential, superposed in part or completely, or form a single sequence.

FIG. 4 is schematic illustrations showing the nucleic acid probe immobilized substrate of the embodiment comprising an electrochemically active substance as a labeling substance, and the nucleic acid probe immobilized substrate of the embodiment wherein a labeling substance is an optically active substance and the covering nucleic acid chain contains a substance for emphasizing a signal emitted from the optically active substance.

FIG. 5 are illustrations showing the nucleic acid probe immobilized substrate of the embodiment.

FIG. 6 is illustrations showing situations of use of the nucleic acid probe immobilized substrate of the embodiment.

FIG. 7 is a plan view showing an array-type nucleic acid probe immobilized substrate of the embodiment.

FIG. 8 is a plan view showing the array-type nucleic acid probe immobilized substrate of the embodiment.

FIG. 9 is illustrations showing parts of the array-type nucleic acid probe immobilized substrate of the embodiment and a detection signal thereby obtained.

FIG. 10 is illustrations showing parts of the array-type nucleic acid probe immobilized substrate of the embodiment and a detection signal thereby obtained.

FIG. 11 is are illustrations showing parts of the array-type nucleic acid probe immobilized substrate of the embodiment and a detection signal thereby obtained.

FIG. 12 is illustrations showing an example of a chip material of the embodiment.

FIG. 13 is illustrations showing an example of a multi-nucleic-acid amplification detection reaction tool of the embodiment.

FIG. 14 is illustrations showing an example of the multi-nucleic-acid amplification detection reaction tool of the embodiment.

FIG. 15 is illustrations showing a use example of the multi-nucleic-acid amplification detection reaction tool of the embodiment.

FIG. 16 is a flowchart showing a target nucleic acid measuring method of the embodiment.

FIG. 17 is an illustration showing an example of a wavelength of an electric signal measured in an embodiment.

FIG. 18 is an illustration showing an example of a situation of use of a nucleic acid probe immobilized substrate of an embodiment.

FIG. 19 is an illustration showing an example of a situation of use of a nucleic acid probe immobilized substrate of the embodiment.

FIG. 20 is a block illustration showing an example of a device for detecting a target nucleic acid of an embodiment.

FIG. 21 is schematic illustrations showing the array-type nucleic acid probe immobilized substrate of the embodiment for electrochemical detection.

FIG. 22 is graphs showing experiment results of Example 1.

FIG. 23 is a graph showing experiment results of Example 1.

FIG. 24 is schematic illustrations showing the array-type nucleic acid probe immobilized substrate of the embodiment for fluorescence detection.

FIG. 25 is a graph showing experiment results of Example 2.

FIG. 26 is a graph showing experiment results of Example 2.

FIG. 27 is a graph showing experiment results of Example 3.

FIG. 28 is a graph showing experiment results of Example 3.

FIG. 29 is a graph showing experiment results of Example 4.

### DETAILED DESCRIPTION

In general, according to one embodiment, embodiments will be hereinafter described with reference to the accompanying drawings. Common constituent elements are denoted by the same numbers or symbols, throughout the embodiments, and duplicated explanations are omitted. Each figure is a schematic illustration for explaining the embodiments and facilitating understandings thereof, and shapes, dimensions, ratios and the like in the figure may be different from the actual shapes, dimensions, ratios and the like, but their designs can be appropriately changed by referring to the following descriptions and publicly known techniques.

### 1. Definition

The term “amplification” means continuously replicating a template nucleic acid replicated using a primer set. The amplification method used in the embodiments may be a method of isothermally amplifying a target nucleic acid, using a primer set. The amplification method may include, for example, PCR amplification, LAMP amplification, RT-LAMP amplification, SMAP amplification, ICAN amplification, etc., but is not limited to these. In addition, a reverse transcription reaction may be conducted simultaneously with the amplification reaction as desired.

The term “target sequence” means a sequence to be amplified by a primer set, and may include a region to which a primer to be used is bonded.

The term “target nucleic acid” means a nucleic acid including the target sequence. The target nucleic acid is a sequence used as a template by a primer set to be used, and is also referred to as a “template nucleic acid”. The target nucleic acid may be a test nucleic acid contained in a sample which is to be subjected to the amplification reaction or may be an amplification product obtained by amplifying the target sequence using a primer set to amplify the target sequence.

The term “primer set” means a set of primers necessary to amplify a target nucleic acid. For example, in the case of a primer set for PCR amplification, one primer set may include one type of forward primer and one type of reverse primer for amplifying one target nucleic acid. For example, in the case of a primer set for LAMP amplification, one primer set may include a FIP primer and a BIP primer for amplifying at least one target nucleic acid, and may include an F3 primer, a B3 primer, an LP primer, i.e., an LF primer and/or LB primer as needed.

The “sample” is a substance containing a target nucleic acid which is brought to the reaction field and which is to be amplified and detected in the field. Examples of the sample may include, for example, blood, serum, leukocyte, urine, feces, semen, saliva, tissue, biopsy, oral mucosa, culture cell, sputum, etc., or may be those obtained by extracting any of the aforementioned samples or a mixture thereof into a nucleic acid component using any means, but are not limited to these.

### 2. First Embodiment

2-1. Method for Detecting a Target Nucleic Acid

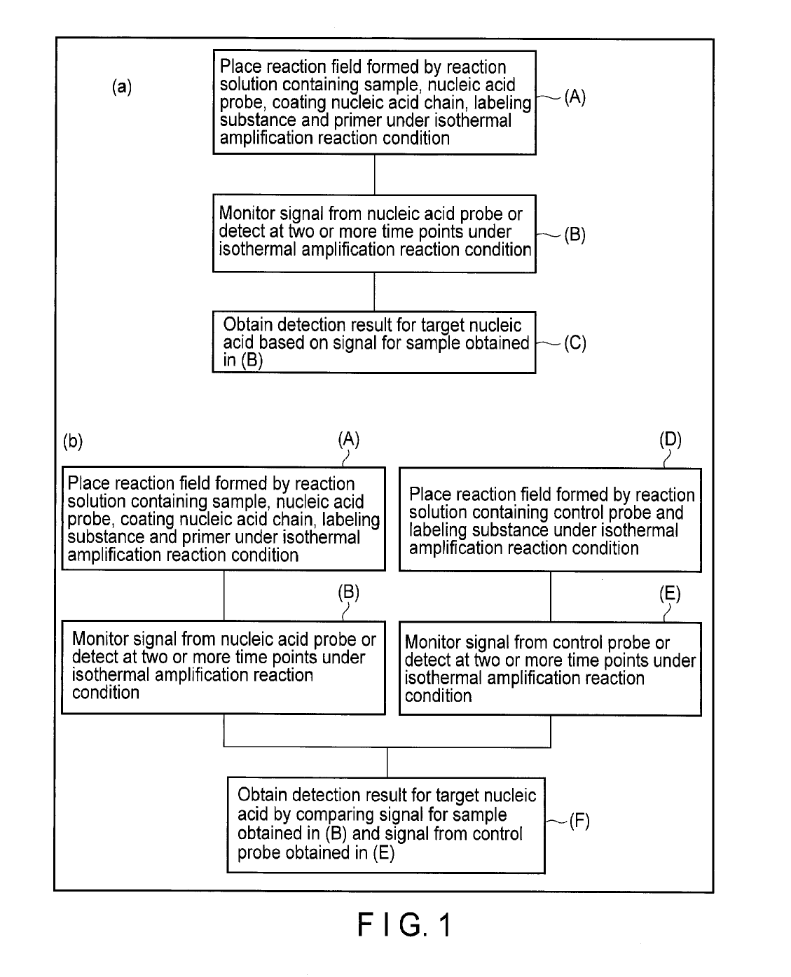

According to a first embodiment, a method for detecting a target nucleic acid is provided. A target nucleic acid to be detected comprises a first sequence and/or a complementary sequence thereof. The method for detecting the target nucleic acid may comprise the following processes (A) to (C) as shown in FIG. 1(a).

In (A), a reaction field formed by a reaction solution containing a sample, a nucleic acid probe, a covering nucleic acid chain, a labeling substance, and a primer set is placed under an isothermal amplification reaction condition. The sample may comprise the target nucleic acid. The nucleic acid probe comprises a nucleic acid chain comprising a second sequence different from the first sequence, thereby being immobilized to at least one surface of a substrate configured to support the reaction field. The covering nucleic acid chain comprises a second sequence bonding region complementary to the second sequence, and a first sequence bonding region complementary to the first sequence. The second sequence bonding region of the covering nucleic acid chain hybridizes to the second sequence of the nucleic acid probe such that the covering nucleic acid chain is bonded to the nucleic acid probe. In the labeling substance which produces a detectable signal, detection of the detectable signal produced by the labeling substance is inhibited by a presence or an increase in abundance of the nucleic acid which is bonded to the nucleic acid probe. A primer set is a primer set configured to amplify the first sequence of the target nucleic acid, and the primer set forms an amplification product comprising the first sequence.

In (B), the signal from the nucleic acid probe is monitored or detected at two or more time points, under the isothermal amplification reaction condition.

In (C), a detection result for the target nucleic acid is obtained based on the signal for the sample obtained in (B).

Here, each of a sequence of the nucleic acid probe and the covering nucleic acid chain have the following characteristics (a) or (b):

(a) a sequence for obtaining competition of the amplification product and the nucleic acid probe to the covering nucleic acid chain, desorption of the covering nucleic acid chain from the nucleic acid probe by the competition, and bonding via hybridization between the first sequence bonding region of the covering nucleic acid chain and the first sequence of the amplification product, under the isothermal amplification reaction condition.

(b) a sequence for obtaining the bonding via hybridization between the first sequence bonding region of the covering nucleic acid chain and the first sequence of the amplification product, and elongation of the covering nucleic acid chain using the amplification product as a template, with the bonding between the nucleic acid probe and the covering nucleic acid chain being maintained, under the isothermal amplification reaction condition.

For example, when each of a sequence of the nucleic acid probe and the covering nucleic acid chain are the sequences of the above (a), lengths and Tm values of the base sequence of the nucleic acid probe and the covering nucleic acid chain are set such that, for example, under the isothermal amplification reaction condition, the bonding via hybridization between the nucleic acid probe and the covering nucleic acid chain is maintained if the target nucleic acid is not present in the reaction field, and that the bonding is eliminated if the target nucleic acid is present in the reaction field and if the target nucleic acid and the nucleic acid probe are competitive to the covering nucleic acid chain.

In addition, for example, when each of the sequence of the nucleic acid probe and the covering nucleic acid chain are the sequences of the above (b), lengths and Tm values of the base sequence of the nucleic acid probe and the covering nucleic acid chain are set such that, for example, under the isothermal amplification reaction condition, the bonding via hybridization between the nucleic acid probe and the covering nucleic acid chain is maintained regardless of both cases where the target nucleic acid is present in the reaction field, and where the target nucleic acid is not present in the reaction field.

In addition, the detection result for the target nucleic acid may also be obtained by comparison with a signal from a control probe. In this case, a method comprising the following processes (D), (E) and (F) may be provided in addition to the above (A) and (B) as shown in FIG. 1(b).

In (D), the reaction field formed by a reaction solution containing a control probe and a labeling substance is placed under the isothermal amplification reaction condition.

In (E), the signal from the control probe is monitored or detected at two or more time points, under the isothermal amplification reaction condition.

In (F), a detection result for the target nucleic acid is obtained by comparing the signal for the sample obtained in (B) and the signal from the control probe obtained in (E).

Such a method is capable of being performed using, for example, the following assay kit and probe immobilized substrate.

2-2. Assay Kit

According to an embodiment, the assay kit may be provided. An example of the assay kit configured to detect the target nucleic acid includes a primer set for amplifying the target nucleic acid, a probe immobilized substrate for performing an isothermal amplification reaction therein and detecting an amplification product obtained by the isothermal amplification reaction, a labeling substance which produces a detectable electrochemical signal, and arbitrarily, a reaction reagent.

An example of the probe immobilized substrate includes a substrate configured to support a reaction field in which an isothermal amplification reaction is performed, a probe immobilization region arranged on at least one surface of the substrate in contact with the reaction field when the reaction field is formed, a nucleic acid probe immobilized to the probe immobilization region and including a nucleic acid chain which includes a second sequence, and a covering nucleic acid chain which includes a first sequence bonding region complementary to a first sequence and a second sequence bonding region complementary to the second sequence, and which is bonded to the nucleic acid probe via hybridization to the second sequence in the second sequence bonding region.

The detection of the detectable signal produced by the labeling substance may be inhibited by a presence or an increase in abundance of the nucleic acid which is bonded to the nucleic acid probe. The labeling substance may be included in the assay kit independently from the probe immobilized substrate, or may be indirectly immobilized or may be releasably and directly immobilized at a position corresponding to the nucleic acid probe on at least one surface of the substrate to which the nucleic acid probe is immobilized.

Another example of the probe immobilized substrate includes a substrate configured to support a reaction field in which an isothermal amplification reaction for amplifying a first sequence and/or a complementary sequence thereof using a primer set to obtain an amplification product is performed, a probe immobilization region arranged on at least one surface of the substrate in contact with the reaction field when the reaction field is formed, a nucleic acid probe immobilized to the probe immobilization region and including a nucleic acid chain which includes a second sequence, and a covering nucleic acid chain which includes a first sequence bonding region complementary to the first sequence and a second sequence bonding region complementary to the second sequence, and which is bonded to the nucleic acid probe via hybridization to the second sequence in the second sequence bonding region, and a labeling substance which produces a detectable signal that is indirectly immobilized or releasably and directly immobilized at a position corresponding to the nucleic acid probe on the surface of the substrate.

2-3. Probe Immobilized Substrate

According to an embodiment, for example, the following probe immobilized substrate is provided.

The substrate may support a reaction field in which an isothermal amplification reaction using first to n-th primer sets produces first to n-th amplification products comprising the 1₁-th to mutually different 1ₙ-th sequences, respectively, with the first to n-th target nucleic acids used as templates, respectively.

As probe immobilization regions, first to n-th probe immobilization regions arranged independently on at least one surface of the substrate in contact with the reaction field when the reaction field is formed may be included.

As the nucleic acid probe, a nucleic acid probe group respectively including first to n-th nucleic acid chains respectively comprising 2₁-th to 2ₙ-th sequences immobilized respectively to the respective first to n-th probe immobilization regions may be included.

As the covering nucleic acid chain, first to n-th covering nucleic acid chains which respectively include 1₁-th to 1ₙ-th sequence bonding regions that are respectively complementary to the respective 1₁-th to 1ₙ-th sequences, and 2₁-th to 2ₙ-th sequence bonding regions that are respectively complementary to the respective 2₁-th to 2ₙ-th sequences, and which are bonded to respective first to n-th nucleic acid probes via hybridization with the respective 2₁-th to 2ₙ-th sequences in the respective 2₁-th to 2ₙ-th sequence bonding regions may be included.

Each of a sequence of the first to n-th nucleic acid probes and the first to n-th covering nucleic acid chains satisfy the following condition of (a) or (b):

(a) a sequence for obtaining respective competitions of the first to n-th amplification products and the first to n-th nucleic acid probe sequences corresponding to the respective first to n-th nucleic acid chains, desorption of the first to n-th covering nucleic acid chains from the respective first to n-th nucleic acid probes by the competitions, and respective bondings via respective hybridizations between the 1₁-th to 1ₙ-th sequence bonding regions of the first to n-th covering nucleic acid chains and the 1₁-th to 1ₙ-th sequences of the first to n-th amplification products, under an isothermal amplification reaction condition in the formed reaction field,

(b) a sequence for obtaining respective bondings via respective hybridizations between the 1₁-th to 1ₙ-th sequence bonding regions of the respective first to n-th covering nucleic acid chains and the 1₁-th to 1ₙ-th sequences of the first to n-th amplification products, and respective elongations of the first to n-th covering nucleic acid chains using the respective first to n-th amplification products as templates, under the isothermal amplification reaction condition in the formed reaction field.

According to the first embodiment as described above, the nucleic acid is capable of being easily detected with high sensitivity.

### 3. Second Embodiment

An example of the method for detecting the target nucleic acid in which the each of the sequence of the nucleic acid probe and the covering nucleic acid chain are the sequences of the above (a) is described below in more detail as the second embodiment.

The method for detecting the target nucleic acid of the second embodiment is a method for detecting a target nucleic acid comprising a first sequence and/or a complementary sequence thereof. The first sequence may be an arbitrary sequence. This method executes the detection of an amplification product which has a signal from a nucleic acid prove as index, simultaneously with an isothermal amplification reaction, in the same reaction field, under the same reaction conditions.

A primer set used for the isothermal amplification may be a primer set for amplification of a first sequence included in the target nucleic acid. In addition, the primer set is preferably designed to include the first sequence at a single-stranded site of the amplification product obtained in the reaction field. For example, if LAMP is applied, a LAMP amplification product has a stem loop structure including a loop site which is a single-stranded region and a stem site which is a double-stranded region. In this case, the stem loop structure can be designed such that the loop site includes the first sequence.

The nucleic acid probe includes a nucleic acid chain immobilized on a solid phase and a labeling substance which is bonded to the nucleic acid chain and which can produce a detectable signal. The nucleic acid chain includes a second sequence different from the first sequence. Such a nucleic acid probe is hybridized with a covering nucleic acid chain in an initial state prior to carrying out the detection method.

The covering nucleic acid chain is a nucleic acid including two sequence regions. One of the regions is a second sequence bonding region. This area has a sequence complementary to the second sequence included in the nucleic acid probe. The covering nucleic acid chain is bonded to the nucleic acid probe by hybridization of this region with the second sequence in the nucleic acid probe. Since the covering nucleic acid chain is bonded to the nucleic acid probe, detection possibility of the signal from the labeling substance in the nucleic acid probe is inhibited. The inhibition of detection possibility of the signal from the labeling substance indicates inhibition of detection or inhibition of detectability such as modifying in a state in which the signal originally produced by the labeling substance cannot be detected or a state in which the signal to be detected when the covering nucleic acid chain is not bonded to the nucleic acid probe cannot be detected. The state indicates that, for example, when the nucleic acid probe including the labeling substance is independently present in a single-stranded form, a detected signal is attenuated, eliminated, or changed or modified to an undetectable signal by the bonding of the covering nucleic acid chain to the nucleic acid probe. Such inhibition of detection possibility of the signal from the labeling substance is irreversible. If bonding the covering nucleic acid to the nucleic acid probe is eliminated, i.e., if the covering nucleic acid is desorbed from the nucleic acid probe, the signal which originally produces the labeling substance can be detected.

The second region included in the covering nucleic acid chain is the first sequence bonding region. This region has a sequence complementary to the first sequence included in the amplification product. The first sequence in the amplification product is hybridized with the first sequence bonding region.

Since the covering nucleic acid chain includes the above-explained two regions, i.e., the region for bonding the nucleic acid probe and the area for bonding the amplification product, a competitive reaction between the amplification product and the nucleic acid probe on the covering nucleic acid chain can be obtained. The covering nucleic acid chain is desorbed from the nucleic acid probe in accordance with abundance of the amplification product, by utilizing the competitive reaction. Then, the sequence of the first sequence bonding region of the covering nucleic acid chain is hybridized with the first sequence in the amplification product. The covering nucleic acid chain is thereby bonded to the amplification product.

In the target nucleic acid detecting method, the isothermal amplification reaction is carried out in the reaction field in which the nucleic acid probe having detectability masked by such bonding of the covering nucleic acid chain. Simultaneously with this, the competitive reaction between the nucleic acid probe and the amplification product produced by the isothermal amplification reaction is made. Simultaneously with this, the signal from the labeling substance produced by the desorption of the covering nucleic acid chain from the nucleic acid probe is detected. The signal detection may be sequentially monitored or may be intermittently executed at a plurality of times.

The nucleic acid probe and the covering nucleic acid chain meet the following two conditions under the isothermal amplification reaction conditions:

(i) when a nucleic acid including the first sequence is not present in the reaction field, bonding between the nucleic acid probe and the covering nucleic acid chain through hybridization is maintained; and

(ii) when a nucleic acid including the first sequence is present in the reaction field, the nucleic acid and the nucleic acid probe become competitive to the covering nucleic acid chain, and bonding between the nucleic acid probe and the covering nucleic acid chain is eliminated.

These conditions can be met by designing a base sequence and a base length in the nucleic acid probe and the covering nucleic acid chain in consideration of a Tm value. Bringing the primer set to the reaction field may be addition of the primer set to the solid phase to which the nucleic acid probe is immobilized, and the amplification product thereby produced may be releasably immobilized to the solid phase so as to meet the nucleic acid probe.

Such a target nucleic acid detecting method can detect the nucleic acid more simply with higher sensitivity. In addition, the target nucleic acid can be quantitatively detected by the method. This method can be carried out with the nucleic acid probe immobilized substrate.

3-1. Nucleic Acid Probe Immobilized Substrate

An example of a nucleic acid probe immobilized substrate will be explained with reference to FIG. 1. The nucleic acid probe immobilized substrate is an example of a reaction tool for detecting a target nucleic acid in a sample by isothermally amplifying the target nucleic acid in the sample and by detecting the amplification product obtained from the amplification.

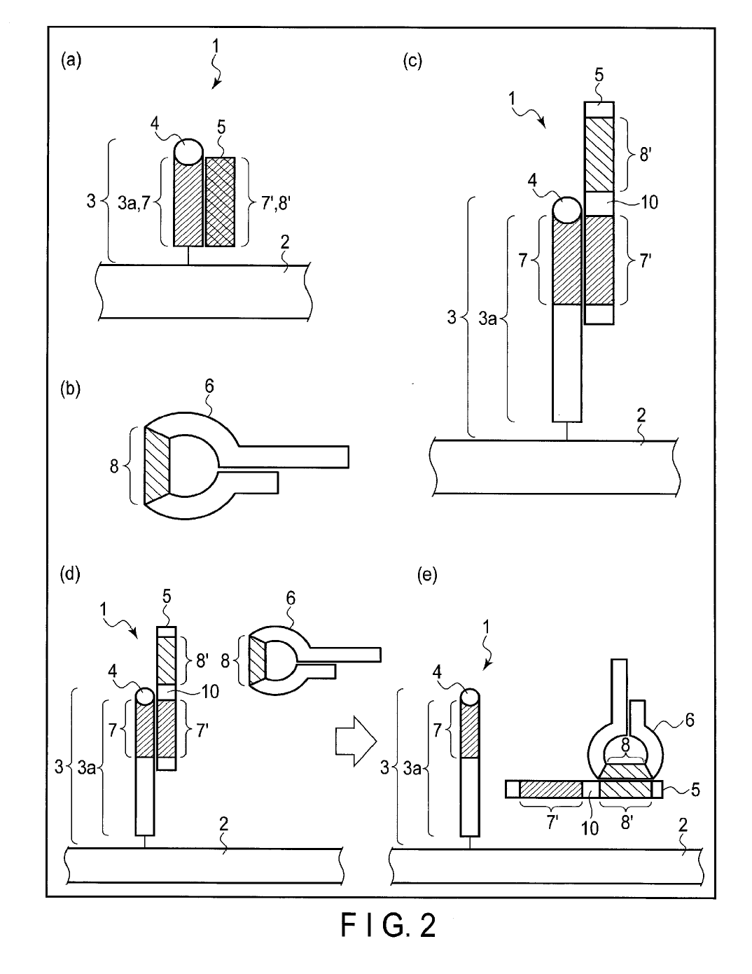

FIG. 2(a) shows an initial state of an example of the nucleic acid probe immobilized substrate. FIG. 2(b) schematically illustrates an example of the amplification product. FIG. 2(c) shows an initial state of another nucleic acid probe immobilized substrate. FIG. 2(d) and FIG. 2(e) are schematic illustrations showing states in which the nucleic acid probe immobilized substrate shown in FIG. 2(c) is used.

As shown in FIG. 2(a), the nucleic acid probe immobilized substrate 1 comprises a substrate 2, a nucleic acid probe 3, and a covering nucleic acid chain 5. The nucleic acid probe 3 comprises a nucleic acid chain 3a immobilized to the substrate 2, and a labeling substance bonded to the nucleic acid chain 3a. FIG. 2(b) illustrates an example of the amplification product obtained from the target nucleic acid which is to be detected by the nucleic acid probe immobilized substrate 1. The amplification product 6 includes a first sequence 8 in a one-dot-chained region. In contrast, the nucleic acid chain 3a of the nucleic acid probe 3 includes a second sequence 7. The covering nucleic acid chain 5 shown in FIG. 2(a) is the sequence of the nucleic acid chain 3a, i.e., a complementary sequence of the second sequence 7 or complementary sequence 8′ of the first sequence 8. In other words, in the covering nucleic acid chain 5, a first sequence bonding region 8′ and a second sequence bonding region 7′ are arranged to be completely superposed, and the sequence of the first sequence bonding region 8′ is equal to the sequence of the second sequence bonding region 7′.

FIG. 2(c) shows another example of the nucleic acid probe immobilized substrate 1. This example has the same structure as the nucleic acid probe immobilized substrate 1 shown in FIG. 2(a) except the sequences in the nucleic acid probe 3 and the covering nucleic acid chain 5. In the present example, the covering nucleic acid chain 5 includes the first sequence bonding region 8′ and the second sequence bonding region 7′ adjacent via another nucleic acid 10 on a nucleic acid chain. In the nucleic acid probe immobilized substrate 1, if the amplification product 6 approaches the nucleic acid probe 3 covered with the covering nucleic acid chain 5 (FIG. 2(d)), the nucleic acid probe 3 and the amplification product 6 become competitive by handling the covering nucleic acid chain 5 as a bonding target, i.e., for the covering nucleic acid chain 5. In FIG. 2(b), an example of the amplification product comprising a stem loop structure including a loop site which is a single-stranded area and a stem site which includes a double-stranded area is shown as the amplification product 6. Thus, bonding between the covering nucleic acid chain 5 and the nucleic acid probe 3 becomes unstable and the amplification product 6 is bonded to the covering nucleic acid chain 5. This bonding is conducted by hybridizing the first sequence with the first sequence bonding region of the covering nucleic acid chain 5. As a result of desorption of the covering nucleic acid chain 5, the signal of the labeling substance 4 included in the nucleic acid probe 3 can be detected. The target nucleic acid in the sample can be measured by using the detectable signal from the labeling substance as an index.

Bonding the labeling substance 4 to the nucleic acid probe 3 may be conducted at any position on the nucleic acid probe 3. In addition, immobilization of the nucleic acid probe 3 to the substrate 2 may be conducted at either the 3′ terminal or the 5′ terminal of the nucleic acid chain 3a. The bonding of the labeling substance 4 may be conducted near a part of the nucleic acid chain 3a bonded to the substrate 2, at or near an unbonded terminal of the nucleic acid chain 3a, or at or near a central part of the nucleic acid chain 3a. A method of bonding the labeling substance 4 to the nucleic acid chain 3a may be selected in accordance with the type of the labeling substance, and any method may be selected for the bonding between the nucleic acid and the labeling substance.

The substrate 2 is constituted to support the reaction field of the liquid phase. The nucleic acid probe 3 immobilized at either of the terminals, on at least one surface of the substrate 2 in contact with the reaction field when the reaction field is formed by the liquid phase. In the drawings, i.e., FIG. 2(a) to FIG. 2(e), the complementary sequences are represented by the same oblique lines. The covering nucleic acid chain 5 shown in FIG. 2(a) is complementary to both the first sequence 8 and the second sequence 7 as represented by cross.

The term “reaction field” means a region defined by the reaction solution where the amplification reaction can proceed theoretically, i.e., a region where the reaction solution exists. Of the reaction field, a region where the amplification reaction actually starts and proceeds is referred to as a “reaction region”. If the amplification reaction actually proceeds in the region alone, the reaction region may be considered as a reaction field.

The substrate 2 may be in a container shape, a plate-like shape, a spherical shape, a rod-like shape and a shape including a part of these shapes. The size and shape of the substrate 2 may be arbitrarily selected by a performer. A base plate having a channel may be used as the substrate 2.

The nucleic acid probe 3 may be immobilized to the substrate 2 after forming the nucleic acid chain 3a included in the nucleic acid probe 3 and bonding the labeling substance 4 to the nucleic acid chain 3a, or the nucleic acid probe 3 may be formed on the substrate and the labeling substance 4 may be bonded to the nucleic acid probe 3. Bonding the covering nucleic acid chain 5 to the nucleic acid probe 3 may be conducted before or after immobilization of the nucleic acid probe 3 to the substrate 2.

The immobilization of the nucleic acid probe 3 to the substrate 2 may be conducted via, for example, a terminal-modified group such as a mercapto group, an amino group, an aldehyde group, a carboxyl group or biotin, but is not limited to these. The selection from these functional groups and the immobilization of the nucleic acid probe 3 can be accomplished by publicly known means.

The length of the nucleic acid probe may be, for example, 3 to 10 bases, 10 to 20 bases, 20 to 30 bases, 30 to 40 bases, 40 to 50 bases or 50 to 60 bases, preferably, 10 to 50 bases.

The covering nucleic acid chain 5 includes the first sequence bonding region 8′ and the second sequence bonding region 7′. The first sequence bonding region 8′ may include the sequence complementary to the sequence in at least a part of the amplification product 6. The second sequence bonding region 7′ may include the sequence complementary to the sequence in at least a part of the nucleic acid probe 3.

As shown in FIG. 2(c), the nucleic acid probe 3 may further include a sequence besides the second sequence 7. In addition, the covering nucleic acid chain 5 may further include a sequence such as a spacer sequence, besides the first sequence bonding region 8′ (complementary sequence of the first sequence 8 of the amplification product 6) and the second sequence bonding region 7′ (complementary sequence of the second sequence 7 of the nucleic acid probe 3).

The covering nucleic acid chain 5 influences the signal emitted from the labeling substance 4 by hybridizing with the nucleic acid probe 3 via the second sequence 7. The hybridization between the covering nucleic acid chain 5 and the nucleic acid probe 3 is eliminated by causing the amplification product 6 and the nucleic acid probe 3 to be competitive to the covering nucleic acid chain 5, desorbing the covering nucleic acid chain 5 from the nucleic acid probe 3, and bonding the covering nucleic acid chain 5 to the amplification product 6 (FIG. 2(e)). Either of the dissociation of the covering nucleic acid chain 5 from the nucleic acid probe 3, and the bonding of the covering nucleic acid chain 5 to the amplification product 6 may first occur, or both of the dissociation and the bonding may occur in parallel.

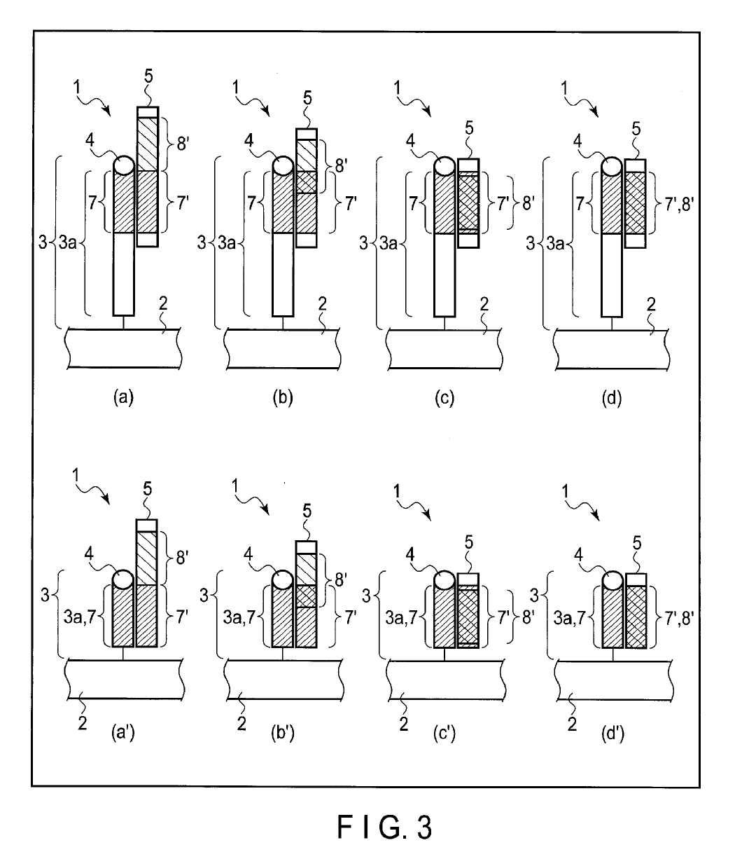

FIG. 3(a) to (d) and (a′) to (d′) show other examples. These examples have the same structure as the nucleic acid probe immobilized substrate 1 shown in FIG. 2(c) except arrangement of the first sequence bonding region 8′ and the second sequence bonding region 7′ of the covering nucleic acid chain 5.

The covering nucleic acid chain 5 may or may not further include a base between the first sequence bonding region 8′ and the second sequence bonding region 7′. FIG. 3(a) and FIG. 3(a′) show examples in which the covering nucleic acid chain 5 does not further include a base between the first sequence bonding region 8′ and the second sequence bonding region 7′, but the regions are arranged adjacent to each other. The first sequence bonding region 8′ and the second sequence bonding region 7′ may be superposed in part on each other (for example, FIG. 3(b) and FIG. 3(b′)). Parts or the entire body of either of the first sequence bonding region 8′ and the second sequence bonding region 7′ may be included in the other region (for example, FIGS. 3(b), (b′), (c) and (c′)). FIGS. 3(c) and (c′) show examples in which the entire body of either of the regions is included in the other region. Alternatively, the first sequence bonding region 8′ and the second sequence bonding region 7′ may be completely superposed to share one sequence (for example, FIG. 2(a), and FIGS. 3(d) and (d′)). The covering nucleic acid chain 5 may include the first sequence bonding region 8′ and the second sequence bonding region 7′ alone (for example, FIG. 2(a)), and may further include bases or base sequences at the 3′ terminal sides and the 5′ terminal sides of the regions (for example, FIG. 2(d), and FIGS. 3(a) to (d) and (a′) to (d′)).

Preferably, the first sequence bonding region 8′ and the second sequence bonding region 7′ of the covering nucleic acid chain 5 should be superposed on each other, but arranged independently of each other, as shown in FIG. 2(c) and FIG. 3(a). In this case, the first sequence bonding region 8′ is used for the hybridization with the amplification product 6 and the second sequence bonding region 7′ is used for the hybridization with the nucleic acid probe 3. Thus, the sequence 7 (i.e., the second sequence) of the nucleic acid probe 3 or the sequence of the second sequence bonding region 7′ of the covering nucleic acid chain 5, which is the complementary chain of the sequence 7, and the sequence (i.e., the first sequence) of the amplification product 6 do not need to be the same sequences. Consequently, the degree of freedom of the nucleic acid probe 3 and the covering nucleic acid chain 5 can be increased and the design can be simplified. The increased degree of freedom and the simplified design are more beneficial when, for example, the nucleic acid probes are used on a nucleic acid probe immobilized substrate as explained later.

The length of the covering nucleic acid chain 5 may be, for example, 3 to 10 bases, 10 to 20 bases, 20 to 30 bases, 30 to 40 bases, 40 to 50 bases, 50 to 60 bases, 60 to 70 bases, 70 to 80 bases, 80 to 90 bases or 90 to 100 bases, preferably, 10 to 50 bases.

The base lengths of the first sequence bonding region 8′ and the second sequence bonding region 7′ may be equal to or different from each other. Preferably, however, affinity (first affinity) between the first sequence bonding region 8′ and the first sequence in the amplification product 6 should be stronger than affinity (second affinity) between the second sequence bonding region 7′ and the nucleic acid probe 3, and should be present more stably after the bonding.

The base lengths of the first sequence bonding region 8′ and the second sequence bonding region 7′ may be independently, for example, 3 to 10 bases, 10 to 20 bases, 20 to 30 bases, 30 to 40 bases, 40 to 50 bases or 50 to 60 bases, preferably 10 to 50 bases. The base lengths of the first sequence bonding region 8′ and the second sequence bonding region 7′ may be equal to or different from each other.

The length of the target sequence may be, for example, 10 to 100 bases, 100 to 200 bases, 200 to 300 bases or 300 to 400 bases, preferably 100 to 300 bases.

The length of the target nucleic acid can be defined by the primer set to be used.

The length of the first sequence in the amplification product may be, for example, 3 to 10 bases, 10 to 20 bases, 20 to 30 bases, 30 to 40 bases, 40 to 50 bases or 50 to 60 bases, preferably 10 to 50 bases.

Bringing the primer set to be used for the isothermal amplification to the reaction field may be conducted by mixing the primer set with a liquid such as a reaction solution and applying the solution to the surface of the substrate on which the nucleic acid probe is immobilized. Alternatively, the primer set may be mixed in the solution such as the reaction solution and the nucleic acid primer immobilized substrate may be dipped in the obtained mixture solution. The primer set may be releasably immobilized on the substrate surface in contact with the reaction field in contact with the substrate surface on which the nucleic acid primer is immobilized, as explained later in detail.

The reaction solution may contain components necessary for a desired amplification reaction. For example, an enzyme such as polymerase, a substrate substance such as deoxynucleoside triphosphate (dNTP) necessary for forming a new polynucleotide chain with a primer used as a start point, a reverse transcriptase and a necessary substrate substance, etc., in a case of performing reverse transcription in parallel, and a buffer such as a salt configured to maintain a proper amplification environment may be included in the reaction solution but the components are not limited to these.

The reaction solution may be a liquid formed by containing, for example, the primer set, a further amplification reagent, for example, an amplification enzyme, dNTP, a buffer, etc., in water. The sample to be tested may be brought to the reaction field in a state of being contained in the reaction solution or may be brought to the reaction field after the reaction solution is carried to the reaction field.

The isothermal amplification reaction conditions such as the temperature and salt concentration of the reaction field are determined in response to selection of the type of the amplification enzyme used in the reaction field. In addition, the lengths and base sequences, of the nucleic acid probe 3 and the covering nucleic acid chain 5 used at the nucleic acid probe immobilized substrate 1 may be designed in response to the type of the selected amplification enzyme, the isothermal amplification reaction conditions such as the temperature and the salt concentration, etc. The nucleic acid probe and the covering nucleic acid chain meet the following two conditions under the isothermal amplification reaction conditions:

(i) when a nucleic acid including the first sequence is not present in the reaction field, bonding between the nucleic acid probe and the covering nucleic acid chain through hybridization is maintained; and

(ii) when a nucleic acid including the first sequence is present in the reaction field, the nucleic acid and the nucleic acid probe become competitive to the covering nucleic acid chain, and bonding between the nucleic acid probe and the covering nucleic acid chain is eliminated.

For example, the Tm value and the sequence length, of the nucleic acid probe 3 and the covering nucleic acid chain 5, are set to maintain hybridization, too, at the salt concentration in the reaction field and the temperature of the amplification reaction.

In association with criteria for designing the nucleic acid probe 3 and the covering nucleic acid chain 5 and determining the isothermal amplification reaction conditions, any of the base sequence lengths of the covering nucleic acid chain and the amplification reactant, and the isothermal amplification reaction conditions such as the temperature and the salt concentration may be first set and then the other conditions may be set to meet the above-explained two conditions.

The salt concentration of the reaction field may be a range in which the amplification reaction can be made, for example, a range of 10 to 120 mM, more preferably, a range of 10 to 60 mM. The temperature of the reaction field in the amplification reaction may be a range in which the amplification reaction can be made, for example, a range of 25 to 70° C., or a range of 55 to 65° C.

For example, the base lengths and the base sequences of the nucleic acid probe and the covering nucleic acid chain should preferably be adjusted such that the temperature condition of the reaction field is a range from 25 to 60° C. and the Tm value of the double-stranded nucleic acid including both the nucleic acid probe and the covering nucleic acid chain by hybridization is 60° C. or higher, as the isothermal amplification reaction conditions.

For example, all the base lengths of the first sequence bonding region 8′, the second sequence bonding region 7′, the first sequence and the second sequence may be the same as or different from each other. The base lengths of the first sequence and the first sequence bonding region may be equal to or different from each other. Additionally or alternatively, the base lengths of the second sequence and the second sequence bonding region may be equal to or different from each other. Furthermore, the base lengths of the first sequence and the second sequence may be equal to or different from each other. Each of the base lengths of the first sequence bonding region 8′, the second sequence bonding region 7′, the first sequence and the second sequence may be selected in accordance with the Tm value under the isothermal amplification reaction conditions.

The base lengths of the nucleic acid probe 3 and the covering nucleic acid chain 5 may be equal to or different from each other. Either of the nucleic acid probe 3 and the covering nucleic acid chain 5 may be longer than the other as shown in, for example, FIG. 2(c) and FIGS. 3(a) to (d). In this case, if the nucleic acid probe 3 and the covering nucleic acid chain 5 are hybridized to form a double strand, either of the 5′ side and the 3′ side is extended to the other of the 3′ side and the 5′ side as a single strand.

If the nucleic acid having the first sequence to be detected, i.e., the target nucleic acid is present in the reaction solution, the primer set present in the reaction field amplifies the target nucleic acid under the isothermal amplification conditions. The amplification product 6 is thereby produced.

The length of the primer may be about 5 bases or more, about 6 bases or more, about 7 bases or more, about 8 bases or more, about 9 bases or more, about 10 bases or more, about 15 bases or more, about 20 bases or more, about 25 bases or more, about 30 bases or more, about 35 bases or more, about 40 bases or more, about 45 bases or more or about 55 bases or more, or may be about 80 bases or fewer, about 75 bases or fewer, about 70 bases or fewer, about 65 bases or fewer, about 60 bases or fewer, about 55 bases or fewer, about 50 bases or fewer, about 45 bases or fewer, about 40 bases or fewer, about 35 bases or fewer, about 30 bases or fewer, about 25 bases or fewer or about 20 bases or fewer, but may be limited to these, or may be in a range of a combination of any of the above-explained upper and lower limits. Preferred examples of the base length may include about 10 to about 60 bases, about 13 to about 40 bases, about 10 to about 30 bases, etc.

The first sequence in the target nucleic acid may be the same as, may be the same in part as, or may be different in part or in an entire length from the sequence of each primer included in the primer set. In a preferred primer set, all the primers included in the primer set have a sequence different from the first sequence.

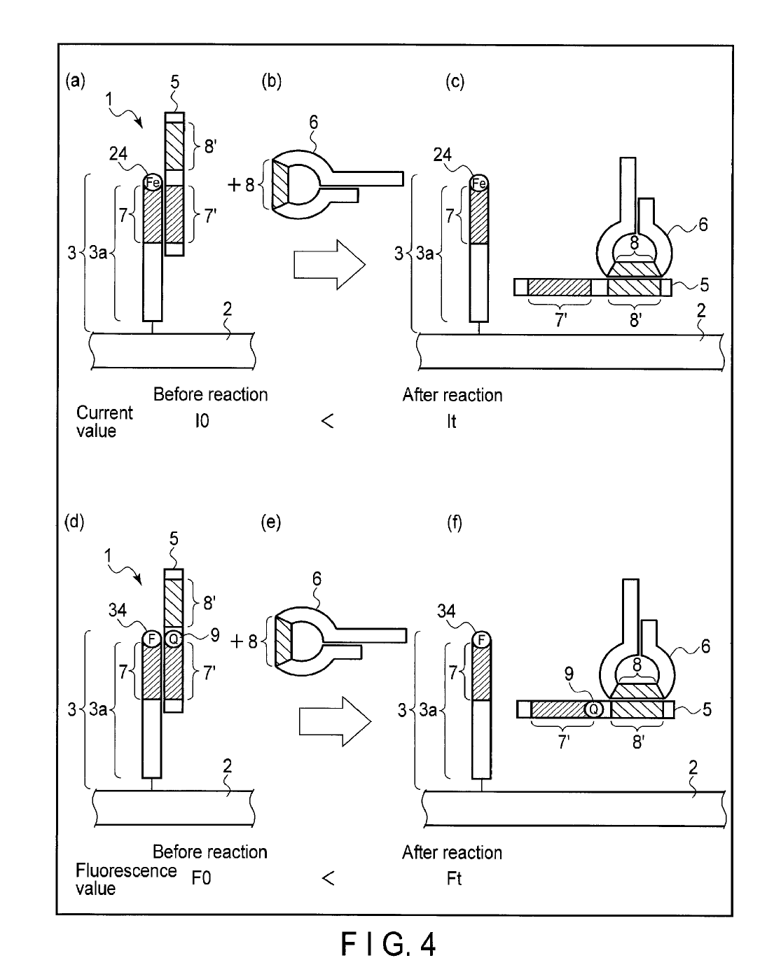

FIGS. 4(a), (b) and (c) are schematic illustrations showing an example of the nucleic acid probe immobilized substrate comprising an electrochemically active substance as the labeling substance 24. This example is structurally the same as that shown in FIG. 2(c) except that the labeling substance 24 is an electrochemically active substance and comprises a sensor for detecting a signal produced by the substance.

As shown in FIG. 4, the nucleic acid probe immobilized substrate 1 of the present example comprises a sensor arranged on the substrate 2 to detect the signal produced by the electrochemically active substance 24, for example, a sensor including an electrode, an interconnect, etc. (not shown). The nucleic acid probe 3 is fixed to the electrode. The double strand including the nucleic acid probe 3 and the covering nucleic acid chain 5 becomes a single strand of the nucleic acid probe 3 as a result of desorbing the covering nucleic acid chain 5 in accordance with presence of the amplification product 6 competitive with the nucleic acid probe 3 (FIGS. 4(b) and (c)). The single strand is the nucleic acid probe 3 immobilized with the substrate 2. As regards the signal from the electrochemically active substance, for example, a current value (I), a current value (Iₜ) obtained when the double strand becomes the single strand due to the presence of the amplification product is greater than a current value (Iₒ) obtained when the corresponding amplification product is not present and the nucleic acid probe 3 and the covering nucleic acid chain 5 are bonded to form the double strand. If the amplification product is present as a result of monitoring for a desired period from the start of the isothermal amplification reaction or measuring at a plurality of times at desired time intervals, a current value greater than a current value in a case where the amplification product is not present can be obtained. Alternatively, rise in increase in the current value can be observed at earlier times. In other words, the magnitude of the signal obtained when the nucleic acid probe 3 is the single strand and the magnitude of the signal obtained when the nucleic acid probe 3 is the double strand need to be different from each other to determine the presence and absence, and the amount of the amplification product. The nucleic acid probe immobilized substrate 1 can detect the target nucleic acid in the sample, simply, with high sensitivity, based on such a difference in signal characteristics. The nucleic acid probe immobilized substrate 1 can quantitatively detect the amplification product present in the reaction field. The nucleic acid probe immobilized substrate 1 can therefore determine the amount of the amplification product in the sample.

The signal from the electrochemically active substance may be, for example, any electronic index such as a current value, a potential value, a capacitance value and an impedance value. Presence and absence or the abundance of the target nucleic acid can be determined by measuring the quantitative variation and/or variation in the predetermined electric characteristic, of the signal involved in the desorption of the covering nucleic acid chain 5 from the nucleic acid probe. The quantitative variation or variation in the electric characteristic, of the signal, may be the variation in magnitude of the signal, for example, reduction or elimination of the signal, length of the time elapsing until these variations in magnitude occur, shift of the start time of the variation in magnitude, variation in a value accumulated within a specific time, etc.

The electrical signal from the nucleic acid probe can be obtained from the substrate onto which the nucleic acid probe is immobilized. In that case, for example, the electrodes may be arranged on at least a part of the substrate surface. In that case, the nucleic acid probe can be immobilized on the electrode.

The electrochemically active substance is not limited to these but, for example, metal complex, iron complex, ruthenium complex, rubidium complex, anthraquinone, methylene blue, etc., which are electrochemically active, can be used as the electrochemically active substance. For example, a compound containing ferrocene can preferably be used. If the electrochemically active substance is used as the labeling substance 4, the labeling substance 4 should preferably be arranged more closely to the sensor than arranged remote from the sensor. Even if the distance from the sensor to the labeling substance 4 is, for example, approximately 50 bases, the electrochemical signal can be detected preferably. The distance from the sensor to the labeling substance 4 may be, for example, 60 bases or fewer, 55 bases or fewer, 50 bases or fewer, 40 bases or fewer, 30 bases or fewer, 20 bases or fewer, or 10 bases or fewer, but is not limited to these. The labeling substance 4 may be arranged in a nucleic acid chain included in the nucleic acid probe, may be imparted to a terminal of a nucleic acid chain which is close to or remote from the base plate, or may be arranged between a terminal-modified base, which bonds a nucleic acid chain in the nucleic acid probe and a substrate, and the nucleic acid chain. Furthermore, a plurality of labeling substances 4 or a single labeling substance 4 may be included in a nucleic acid probe. If the labeling substances 4 are included, they may be in the same type or different types.

In addition, an optically active substance may be used as the labeling substance 4. FIGS. 4(d), (e) and (f) are schematic illustrations showing an example of the nucleic acid probe immobilized substrate comprising an optically active substance as the labeling substance. This example is structurally the same as that shown in FIG. 2(c) and FIGS. 4(a) to (c) except that an optically active substance is used as the labeling substance and that the covering nucleic acid chain 5 includes a quencher 9. The quencher 9 is used to detect an optical signal from the optically active substance more effectively.

As shown in FIG. 4(a), the nucleic acid probe immobilized substrate 1 comprises the substrate 2, the nucleic acid probe 3 bonded to the substrate 2, and the covering nucleic acid chain 5 hybridized with the nucleic acid probe 3, before the nucleic acid probe immobilized substrate 1 is used or when the amplification product is not present. The nucleic acid probe 3 comprises the nucleic acid chain 3a immobilized to the substrate 2 at a terminal thereof, and the optically active substance 34 which is the labeling substance 4 at the other terminal. The covering nucleic acid chain 5 includes a sequence complementary to the sequence 7 (second sequence 7) at a part of the nucleic acid chain 3a (i.e., a sequence of the second sequence bonding region 7′), a sequence complementary to the sequence 8 (first sequence 8) at a part of the amplification product 6 (i.e., a sequence of the first sequence bonding region 8′), and the quencher 9 arranged between the second sequence bonding region 7′ and the first sequence bonding region 8′.

The double strand including the nucleic acid probe 3 and the covering nucleic acid chain 5 becomes a single strand of the nucleic acid probe 3 as a result of desorbing the covering nucleic acid chain 5 in the presence of the amplification product 6 competitive with the nucleic acid probe 3 (FIGS. 4(e), (f)). Detectability of the signal from the optically active substance included in the nucleic acid probe 3 is inhibited by bonding of the covering nucleic acid chain 5. In this example, the covering nucleic acid chain 5 further includes the quencher 9 to reinforce the inhibition.

As regards the signal from the optically active substance, for example, a fluorescence value (F), a fluorescence value (Fₜ) obtained when the double strand becomes the single strand due to the presence of the amplification product is greater than a fluorescence value (Fₒ) obtained when the corresponding amplification product is not present and the covering nucleic acid chain 5 is bonded to the nucleic acid probe 3 to form the double strand. If the amplification product is present as a result of monitoring for a desired period from the start of the isothermal amplification reaction or measuring at a plurality of times at desired time intervals, a fluorescence value greater than a fluorescence value in a case where the amplification product is not present can be obtained. In addition, rise in increase in the fluorescence value can be observed at earlier times. In other words, the signal characteristic obtained when the nucleic acid probe 3 is the single strand and the signal characteristic obtained when the nucleic acid probe 3 is the double strand need to be different from each other to determine the presence and absence, and the amount of the amplification product. The nucleic acid probe immobilized substrate 1 can detect the target nucleic acid in the sample, simply, with high sensitivity, based on such a difference in signal characteristics. The nucleic acid probe immobilized substrate 1 can quantitatively detect the amplification product present in the reaction field. The nucleic acid probe immobilized substrate 1 can therefore determine the amount of the amplification product in the sample.

The signal from the optically active substance may be any optical index, for example, light of a specific wavelength such as fluorescence and light emission. Presence and absence or the abundance of the target nucleic acid can be determined by measuring the variation in quantitative and/or predetermined optical characteristic, of the signal involved in the desorption of the covering nucleic acid chain 5 from the nucleic acid probe. The variation in quantitative or optical characteristic of the signal may be, for example, the variation in light intensity, increase, attenuation and elimination in light intensity, variation in wavelength, etc., or may be the magnitude of the light intensity, length of the time elapsing until the variation in wavelength occurs, shift of the start time of the variation, etc., or may be the variation in a value accumulated within a specific time.

Examples of the fluorescence substance used as the labeling substance are not limited these but include, for example, Alexa flour, BODIPY, Cy3, Cy5, FAM, Fluorescein, HEX, JOE, Marina Blue (trademark), Oregon Green, Pacific Blue (trademark), Rhodamine, Rhodol Green, ROX, TEMRA, TET, Texas Red (registered trademark), etc.

Examples of the quencher contained in the covering nucleic acid chain 5 include BHQ-1, BHQ-2, Dabcyl, etc. For example, if Cy3 or Cy5 is selected as the labeling substance 4, Eu chelate or Ulight may be used as the quencher.

In the above-explained example, the covering nucleic acid chain 5 contains the quencher 9. By containing the quencher 9 in the covering nucleic acid chain 5, the production of the signal from the optically active substance is further suppressed as compared with a case where the covering nucleic acid chain 5 alone is bonded to the nucleic acid probe 3. That is, the difference between the signal value in the state in which the nucleic acid probe 3 and the covering nucleic acid chain 5 are bonded to form the double strand, and the signal value in the state in which the covering nucleic acid chain 5 is desorbed from the covering nucleic acid chain 5 becomes great. In other words, the difference between the signal value in the case where the amplification product 6 is present and the signal value in the case where the amplification product 6 is not present becomes great. The target nucleic acid can be thereby detected with further higher accuracy.

In the nucleic acid probe immobilized substrate, a modifying substance for enhancing or assisting the inhibition effect of signal detectability with the covering nucleic acid chain 5 may be contained in the covering nucleic acid chain 5, similarly to the above-explained quencher. Such a modifying substance may be a substance enhancing or assisting the inhibition of detectability of the signal inherent to the labeling substance, which is inhibited by the bonding of the covering nucleic acid chain 5 to the nucleic probe. For example, such a modifying substance may be a substance enhancing masking, reduction or elimination of the signal from the modifying substance 4 by bonding of the covering nucleic acid chain 5 and/or a substance changing or modifying the signal characteristic of the modifying substance 4 such that it cannot be detected. For example, if an electrochemically active substance is used as a labeling substance, the modifying substance may be a substance enhancing or assisting the reduction or elimination of the electric signal with the covering nucleic acid chain 5. For example, if an optically active substance is used as a labeling substance, the modifying substance may be a substance reducing the optical signal inherently produced by the covering nucleic acid chain 5 and/or changing the wavelength of the optical signal. In other words, the covering nucleic acid chain 5 can increase the variation amount of the signal characteristic of the labeling substance in accordance with the abundance or absence of the amplification product when used together with the modifying substance rather than used by itself. An existence state of the amplification product 6 can be therefore represented with higher accuracy by using of the modifying substance.

As explained above, the primer set is releasably immobilized to the substrate, and may be released to the reaction field when a liquid is brought to form the reaction field. The primer set is brought to the reaction field by the release to the reaction field.

The nucleic acid probe immobilized substrate comprising such an immobilized primer set can further comprise a primer-immobilized region which is arranged on at least one surface of the substrate in contact with the reaction field when the reaction field is formed, and a primer set which is releasably immobilized in the primer-immobilized region. The primer-immobilized region may be arranged on at least one surface of the substrate in contact with the reaction field where a corresponding nucleic acid probe is present.

The nucleic acid probe immobilized substrate may comprise plural types of nucleic acid probes immobilized to the single substrate. When the reaction field is formed in a case where plural types of nucleic acid probes 3 and/or covering nucleic acid chains 5 are used in the single nucleic acid probe immobilized substrate, a plurality of probe-immobilized regions 13 that are arranged independently of each other should preferably be arranged on at least one surface of the substrate 2 in contact with the reaction field.

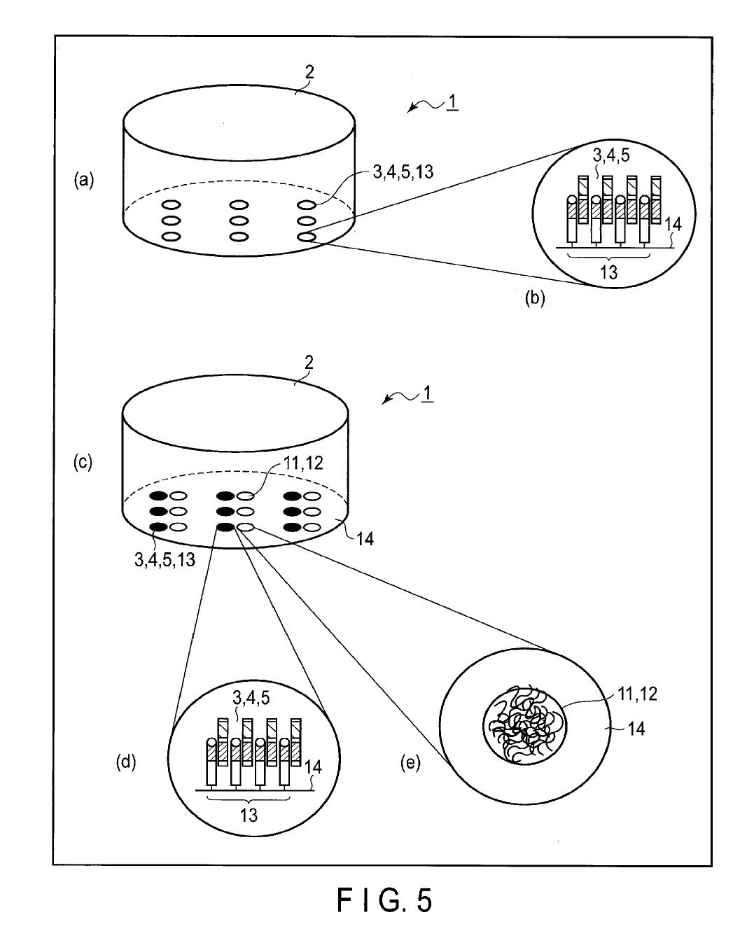

FIG. 5(a) and FIG. 5(c) are perspective views showing another example of the nucleic acid probe immobilized substrate of the embodiment.

The nucleic acid probe immobilized substrate 1 shown in FIG. 5(a) comprises a substrate 2 in container shape. A plurality of probe-immobilized regions 13 that are independent of each other are arranged on an inner bottom surface 14 of the substrate 2. A plurality of double-stranded nucleic acid probes 3 that comprise labeling substances 4 and nucleic acid chains and are bonded to the covering nucleic acid chains 5 are immobilized in the probe-immobilized regions 13. FIG. 5(b) shows a situation of the probe-immobilized region 13 of the nucleic acid probe immobilized substrate 1 shown in FIG. 5(a).

Examples of the container-shaped substrate 2 may include, for example, a tube, a well, a chamber, a channel, a cup and a dish, a plate having a plurality of these forms, such as a multi-well plate, etc. The material of the substrate 2 may be a material that is not itself involved in a reaction, and enables an amplification reaction to be carried out therein. The material may be arbitrarily selected from, for example, silicon, glass, a resin, a metal, etc. Any commercially available container may be used as the container-shaped substrate 2.

The nucleic acid probes 3 immobilized in the probe-immobilized regions 13 arranged in the single substrate 2 may be in the same type over the entire regions, arbitrary regions may be the nucleic acid probes 3 of the same type, and all the regions may be the nucleic acid probes 3 of different types. In addition, the covering nucleic acid chain 5 bonded to the nucleic acid probes 3 may be the same for the nucleic acid probes 3 in all the regions, arbitrary plural regions may be the same as each other or different for each type of the nucleic acid probes 3, and different covering nucleic acid chains 5 may be allocated to all the regions. The nucleic acid probes 3 including covering nucleic acid chains 5 included in one nucleic acid probe immobilized substrate 1 may be nucleic acid probes for detecting an amplification product of one type or plural amplification products of different types. The nucleic acid probes of the same type have the same base length and the same base sequence. Two nucleic acid probes of different types may have different sequences of the same base length, partially similar sequences of different base lengths, or different sequences of different base lengths.

For example, the sequences of the first sequence bonding region 8′ of the covering nucleic acid chain 5 may be different for respective types of the first sequences 8 included in the amplification product 6. Alternatively, the common first sequence bonding region 8′ may be selected for the amplification products of mutually different types that should be detected by one nucleic acid probe immobilized substrate. The common first sequence bonding region 8′ may be selected for several amplification products of the amplification products of plural types that should be detected. Furthermore, plural types of the sequences of the covering nucleic acid chain 5 to be used may be orthonormal sequences. As regards the sequences of the second sequence bonding region 7′ of the covering nucleic acid chain 5, the relationship with the nucleic acid probe and the sequences may be selected similarly.

The orthonormal sequences represent a sequence group having a specific relationship. The sequences are mutually different sequences designed such that the Tm values are uniform, i.e., the Tm values fall within a certain range. They do not inhibit hybridization with the complementary sequence since the nucleic acid molecules themselves are not structured in molecules. They are sequences which do not form stable hybridization with sequences other than the complementary base sequence. Use of such orthonormal sequences is also preferable.

For example, the distance between adjacent probe fixing regions 13 may be 0.1 to 1 μm, 1 to 10 μm, 10 to 100 μm, 100 μm to 1 mm, 1 to 10 mm or more, or may be preferably 100 μm to 10 mm.

Plural types of primer sets may be used in one nucleic acid probe immobilized substrate 1. For example, plural types of primer sets may be contained in a liquid for forming the reaction field, an aqueous solution containing at least a buffer, or a reaction solution further containing a reaction reagent and/or a sample, and brought to the reaction field or a desired substrate surface. Alternatively, plural types of primer sets may be releasably immobilized to the substrate 2 and brought to the reaction field. For example, when plural types of target nucleic acids are detected, plural types of primer sets, nucleic acid probes 3 and/or covering nucleic acid chains 5 can be used.

The nucleic acid probe immobilized substrate to which a primer set is immobilized can contain a substrate, a primer set immobilized to be independently releasable on at least one of surfaces of the substrate, a nucleic acid probe which is independently immobilized to correspond to the primer set and includes a labeling substance producing a detectable signal, and a covering nucleic acid chain which is bonded to the nucleic acid probe 3 and thereby inhibits detectability of the signal from the nucleic acid probe.

When a reaction field is formed in a case of using plural types of primer sets, a plurality of primer-immobilized regions should preferably be arranged independently of each other on at least one surface of the substrate 2 in contact with the reaction field.

FIG. 5(c) is a perspective view showing an example of the nucleic acid probe immobilized substrate immobilizing and comprising the nucleic acid probes and primer sets. In this example, the primer-immobilized regions 11 are arranged in close vicinity to respective probe-immobilized regions 13. FIG. 5(d) shows a situation of the probe-immobilized region 13 of the nucleic acid probe immobilized substrate 1 shown in FIG. 5(c). A plurality of nucleic acid probes 3 labeled by the labeling substance 4 and bonded to the covering nucleic acid chains 5 are immobilized in the probe-immobilized region 13. FIG. 5(e) is an enlarged view showing a situation of the primer-immobilized region 11. The nucleic acid chains, which are plural primers, are immobilized in the primer-immobilized region 11.

The nucleic acid probe immobilized substrate shown in FIG. 5(c) has the same structure as the nucleic acid probe immobilized substrate 1 shown in FIG. 5(a) except immobilization of the primer sets 12 together with the nucleic acid probes 3. A plurality of primer-immobilized regions 11 independent of each other are arranged on an inner bottom surface of the substrate 2 to correspond to a plurality of probe-immobilized regions 13 independent of each other, respectively. The primer sets 12 are immobilized in respective primer-immobilized regions 11.

The primer sets 12 may be immobilized to the substrate 2 in a state of being releasable in contact with a liquid phase for providing a reaction field. For example, immobilization of the primer sets 12 to the substrate 2 can be achieved by dropping a solution containing the primer sets 12 to the substrate 2 and then drying the primer sets 12. In the method of dropping the solution containing the primer sets 12, the solution containing the primer sets 12 may be, for example, water, a buffer solution or an organic solvent. The solution containing desired types of primer sets 12 is dropped to the plurality of primer-immobilized regions 11 and the primer sets are dried. The primer sets 12 are thereby releasably immobilized to the plurality of or all the primer-immobilized regions 11 arranged independently on one of surfaces of the substrate 2.

The immobilization of the primer sets 12 to the substrate 2 may be executed before or after immobilization of the nucleic acid probes 3.

A plurality of primer sets 12 can be immobilized in plural sets, in the single primer-immobilized region 11. A plurality of primer sets 12 can be immobilized to a plurality of primer-immobilized regions 11, respectively, in each type.

Plural types of primer sets 12 can be prepared to amplify a plurality of target nucleic acids, respectively. For example, one type of primer set 12 for amplifying one specific target nucleic acid can be immobilized in plural sets, in one primer-immobilized region 11. For example, In the case of a reaction tool for LAMP amplification, one primer-immobilized region 11 can include a plurality of FIP primers and BIP primers that are necessary to amplify one type of specific target nucleic acid, and can include a plurality of F3 primers, B3 primers and LP primers as needed.

The term “independently arranged” in the immobilized region means that the immobilized regions are arranged at such intervals that amplification made to start and/or proceed for each primer set in a reaction field is not hindered. For example, adjacent immobilized regions may be arranged in contact with each other, or may be arranged in close vicinity of each other with a short distance therebetween, or may be arranged at an interval equivalent to a distance between probes immobilized in a detection reaction tool such as so called a DNA chip which is usually used.

For example, the distance between the adjacent primer-immobilized regions 11 may be 0.1 to 1 μm, 1 to 10 μm, 10 to 100 μm, 100 μm to 1 mm, 1 to 10 mm or more, or may be preferably 100 μm to 10 mm.

The liquid phase for providing a reaction field may be a liquid phase in which after immobilized primer sets 12 are separated, an amplification reaction can be caused to proceed using the primer sets and may be, for example, a reaction solution necessary for desired amplification.

For example, the distance between the probe-immobilized region 13 and the primer-immobilized region 11 may be 0 to 0.1 μm, 0.1 to 1 μm, 1 to 10 μm, 10 to 100 μm, 100 μm to 1 mm, 1 to 10 mm or more, or may be preferably 100 μm to 10 mm.

For example, if the distance between the probe-immobilized region 13 and the primer-immobilized region 11 is 0 μm, the probe-immobilized region 13 and the primer-immobilized region 11 may be considered to be at the same position on the surface of the substrate 2. The probe-immobilized region 13 may be included in the primer-immobilized region 11 or the primer-immobilized region 11 may be included in the probe-immobilized region 13.

The number of types of primer sets 12 immobilized in one primer-immobilized region 11 may be one for amplifying one type of target nucleic acid or may be plural, for example, at least two for amplifying at least two types of target nucleic acids, respectively. Thus, a plurality of primer sets 12 immobilized in one primer-immobilized region 11 may be different from each other or may be partially different in sequence from or partially the same in sequence as each other as desired. The lengths of primers immobilized to one substrate 2 may be the same as each other for each primer or may be different from each other for every primer, or some of the primers may be the same in length as or different in length from each other. The lengths may be different for each primer set or types of the primers included in the primer set.

The numbers of the primer-immobilized regions 11 and the probe-immobilized regions 13 arranged on one nucleic acid probe immobilized substrate 1 may be the same as or different from each other. That is, the probe-immobilized regions 13 which are the same in number as the primer-immobilized regions 11 may be arranged to correspond to all the primer-immobilized regions 11, the number of primer-immobilized regions 11 may be larger than the number of probe-immobilized regions 13, or the number of primer-immobilized regions 11 may be smaller than the number of probe-immobilized regions 13.

The nucleic acid probe immobilized substrate 1 may further include a positive control and/or a negative control for checking a positive signal and/or a negative signal. The positive control and the negative control can be immobilized in a control-immobilized region. The positive control and/or negative control may be provided for the primer set 12 and/or nucleic acid probe 3.

The positive control can use, for example, a labeled single-stranded probe. The negative control can use, for example, a double-stranded probe having no sequence complementary to the amplification product.

In FIG. 5(a) and FIG. 5(c), the probe-immobilized regions 13 and the primer-immobilized regions 11 are arranged on the inner bottom surface of the substrate 2, but are not limited to this example, and may be arranged on at least a part of the inner side surface of the substrate 2, or may be arranged on any or all of the inner bottom surface and the inner side surface such as the ceiling surface defined by the cover attached to the substrate 2.

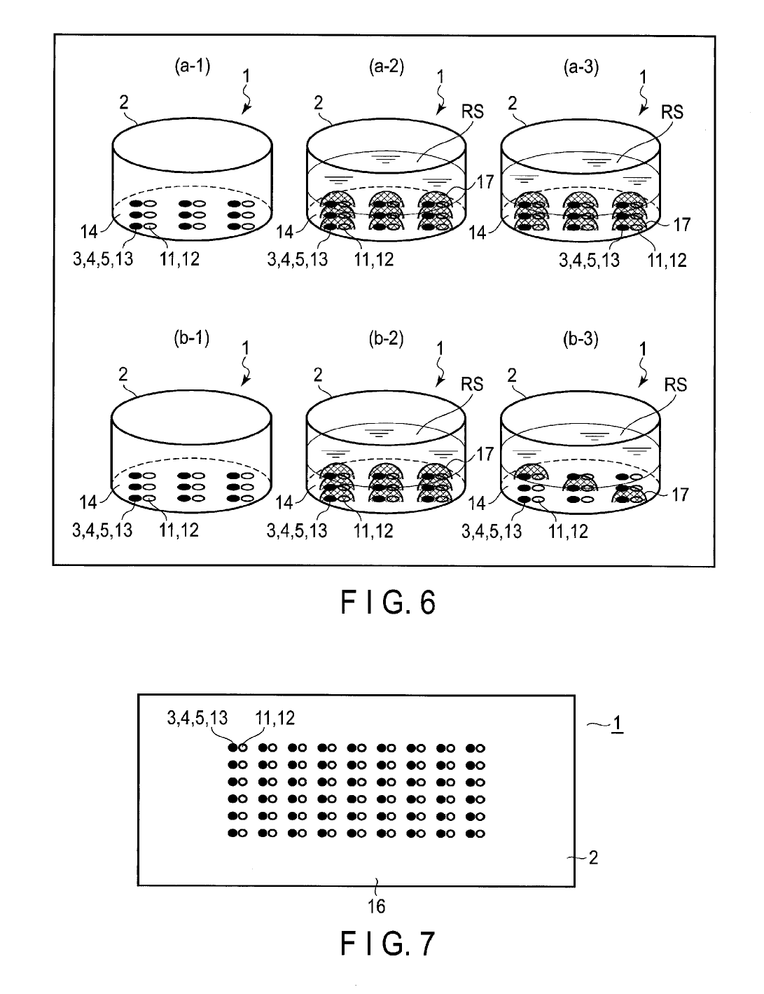

Situations of use of the nucleic acid probe immobilized substrate 1 shown in FIG. 5(c) will be explained with reference to FIG. 6. FIG. 6 is a schematic illustration showing over time situations of a nucleic acid reaction executed in the container-shaped nucleic acid probe immobilized substrate 1.

FIG. 6(a-1) and FIG. 6(b-1) show the nucleic acid probe immobilized substrate 1 set prior to a reaction. A plurality of primer sets 12 are immobilized on a plurality of primer-immobilized regions 11 arranged on the inner bottom surface 14 of the substrate 2, respectively. The probe-immobilized regions 13 are arranged in close vicinity of the respective primer-immobilized regions 11 so as to correspond to the respective primer-immobilized regions 11. A plurality of nucleic acid probes 3 are immobilized for each desired type, in the probe-immobilized regions 13, and the covering nucleic acid chains are hybridized to the plurality of nucleic acid probes 3.

FIG. 6(a-2) and FIG. 6(b-2) show a state in which a reaction solution RS is added and stored in the nucleic acid probe immobilized substrate 1.

The addition of the sample to the inside of the substrate 2 may be executed by preliminarily adding the sample to the reaction solution RS prior to adding the reaction solution RS to the nucleic acid probe immobilized substrate 1. Alternatively, it may be executed by adding the reaction solution RS to the nucleic acid probe immobilized substrate 1 and then adding the sample to the reaction solution RS. It may be executed by adding the sample to the nucleic acid probe immobilized substrate 1 prior to adding the reaction solution RS to the nucleic acid probe immobilized substrate 1.

As shown in FIG. 6(a-2) and FIG. 6(b-2), the primer sets 12 immobilized on the inner bottom surface 14 are released and gradually diffused, in the nucleic acid probe immobilized substrate 1, after the reaction solution RS is added. A region where the primer sets are released and diffused is schematically illustrated by a region. The released and diffused primer sets 12 encounter other components necessary for amplification of a template nucleic acid, polymerase, a substrate substance, etc. that exist in close vicinity, and an amplification reaction is started. A plurality of primer sets 12 independently immobilized for each type can cause the amplification reaction to start and proceed for the template nucleic acid independently for each type. The amplification for a plurality of template sequences using plural types of primer sets 12 is thereby achieved independently in parallel.

FIG. 6(a-3) and FIG. 6(b-3) schematically illustrate a state in which the template nucleic acid to be amplified is present, and the amplification reaction occurs and proceeds in the region where the primer sets are released and diffused.

FIG. 6(a-3) schematically illustrates a region where the amplification reaction occurs by the primer sets 12 immobilized on all the primer-immobilized regions 11 and proceeds, as the reaction region. The amplification occurs in some of all the primer fixing regions 11 where the primer sets are immobilized on the inner bottom 14, i.e., three regions alone, in FIG. 6(b-3), and the regions where the amplification proceeds are schematically illustrated as the reaction regions in FIG. 6(b-3).

FIG. 7 shows a chip-type nucleic acid probe immobilized substrate, i.e., the array-type of the embodiment. The array-type nucleic acid probe immobilized substrate 1 shown in FIG. 7 is an example using a base plate as the substrate 2. A plurality of primer-immobilized regions 11 are arranged independently of each other, on a surface 16 of the substrate 2. In the primer-immobilized regions 11, one type of primer set 12 is immobilized in one primer-immobilized region 11, similarly to the example shown in FIG. 5(c). A plurality of primer sets 12 are immobilized, for each type, in the plurality of primer-immobilized regions 11, respectively. The primer set 12 included in one primer-immobilized region 11 can include, for example, different types of primers necessary to amplify one type of specific target nucleic acid.

Probe-immobilized regions 13 are arranged in close vicinity of the primer-immobilized regions 11 so as to correspond to the primer-immobilized regions 11, respectively. A plurality of nucleic acid probes 3 are immobilized for each desired type, in the probe-immobilized regions 13, and the covering nucleic acid chains 5 are hybridized to the nucleic acid probes 3.

The amplification and detection of the nucleic acid using the nucleic acid probe immobilized substrate 1 can be executed by placing a reaction solution on at least a region or a surface of the substrate 2 on which the primer sets 12 and the nucleic acid probes 3 are immobilized to form a reaction field.

Alternatively, the nucleic acid probe immobilized substrate 1 can be arranged inside the container when the amplification and detection of the nucleic acid are executed. The reaction field can be formed by adding a reaction solution in the container. In this case, the primer sets 12 and the nucleic acid probes 3 can be immobilized on both surfaces of the substrate 2. More types of primer sets 12 and nucleic acid probes 3 can be thereby immobilized on the substrate 2 of the nucleic acid probe immobilized substrate 1. Consequently, more target sequences can be amplified and detected.

Furthermore, a label to distinguish positions of the primer sets 12 and/or nucleic acid probes 3 on the nucleic acid probe immobilized substrate 1 can be imparted to the substrate 2. Impartment of the label can be executed by means which is publicly known itself.

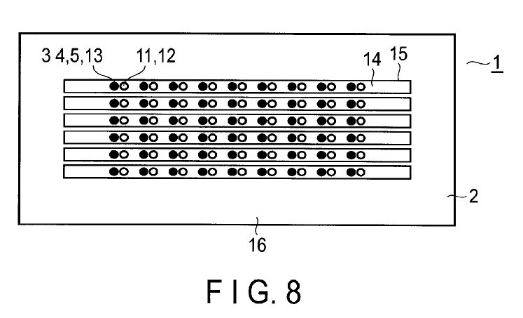

Another example of the nucleic acid probe immobilized substrate will be explained with reference to FIG. 8.

FIG. 8 is a plan view showing the array-type nucleic acid probe immobilized substrate. The array-type nucleic acid probe immobilized substrate 1 shown in FIG. 8 is an example using a base plate comprising a channel as a substrate 2. A plurality of channels 15 composed of a plurality of grooves extended linearly and arranged parallel to each other are formed on a surface 16 of the substrate 2. A plurality of primer-immobilized regions 11 are arranged independently of each other, on a bottom portion 16 of each of the channels 15, along a longitudinal direction of the channel 15. In the primer-immobilized regions 11, one type of primer set 12 is fixed on one primer-immobilized region 11, similarly to the example shown in FIG. 5(c). A plurality of primer sets 12 are immobilized, for each type, in the plurality of primer-immobilized regions 11, respectively. The primer set 12 included in one primer-immobilized region 11 may include, for example, different types of primers necessary to amplify one type of specific target nucleic acid.

Probe-immobilized regions 13 are arranged in close vicinity of the primer-immobilized regions 11 so as to correspond to the respective primer-immobilized regions 11. In each channel 15, the primer-immobilized regions 11 and the probe-immobilized regions 13 are arranged alternately, along a longitudinal direction of the channel 15. A nucleic acid probe which includes a labeling substance 4 and is bonded to a covering nucleic acid chain 5 is immobilized in one probe-immobilized region 13. The nucleic acid probe for detecting mutually different target nucleic acids can be immobilized in each of the plurality of probe-immobilized regions 13.

The amplification and detection of the nucleic acid according to the present embodiment can be executed similarly to the embodiment shown in FIG. 7. Alternatively, the reaction field may be formed and the amplification and detection of the nucleic acid may be executed by allowing a fluid to flow in the channel 15. Furthermore, a trench forming the channel 15 may be covered with a desired lid (not shown).

The above-explained nucleic acid probe immobilized substrate 1 may be produced by forming channels on the surface of the substrate 2, immobilizing the primer sets 12 and the nucleic acid probes 3 on at least one wall surface in each of the formed channels, and hybridizing the covering nucleic acid chains 5 to the nucleic acid probes 3.

Formation of the channels 15 may be executed by forming recess portions or protruding portions, or both the recess portions and the protruding portions on a surface of the substrate 2. The shape of the channels 15 can be thereby defined by the recess portions or protruding portions, or both the recess portions and the protruding portions. For example, the formation of the channels 15 can be executed by subjecting the surface of the substrate 2 to means for forming a trench on the base plate, which is publicly known itself, such as etching. The number of channels 15 included in the substrate 2 may be one or plural, preferably, plural.

Arrangement of the primer-immobilized regions 11 and the probe-immobilized regions 13, and immobilization of the primer sets 12 and the nucleic acid probes 3 may be executed, similarly to the above-explained embodiment.

The positions of the primer-immobilized regions 11 and the probe-immobilized regions 13 arranged for the channels 15 are not limited to the channel bottom surface alone, but may be on any surfaces of the channel. For example, such a surface may be the bottom surface, side surfaces and/or an arbitrary ceiling surface of the channel 15. The ceiling surface of the channel 15 can be, for example, a ceiling surface of the channel 15 provided by attaching a cover or a lid configured to cover all the channels 15 or each channel 15 independently, on the substrate 2.

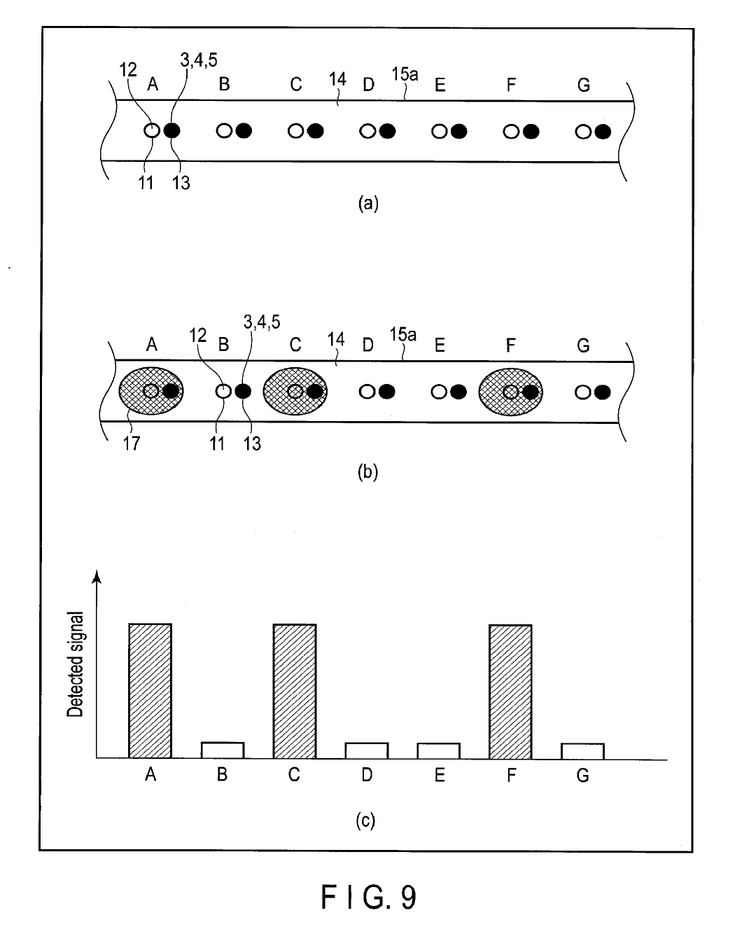

Situations of the amplification reaction and detection results, in the array-type nucleic acid probe immobilized substrate 1 in which the primer-immobilized regions 11 and the probe-immobilized regions 13 are arranged in the channels 15, will be explained with reference to FIG. 9 to FIG. 11.

FIG. 9 shows one of the channels 15 formed in the array-type nucleic acid probe immobilized substrate 1 shown in FIG. 8.

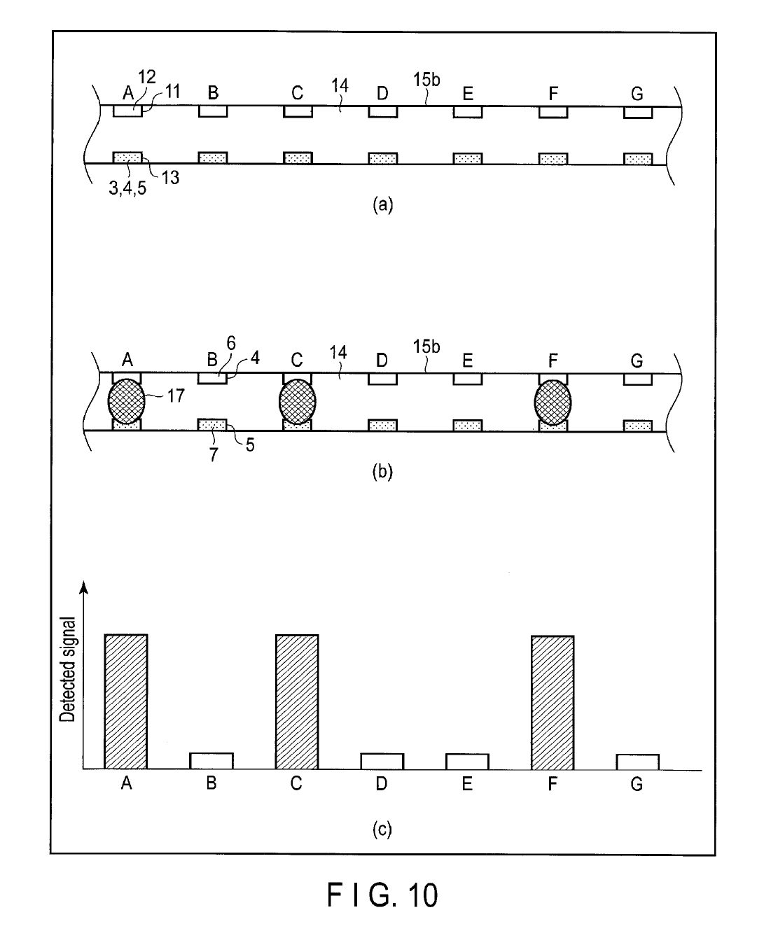

FIG. 10 shows a channel in a nucleic acid probe immobilized substrate similar to the array-type nucleic acid probe immobilized substrate 1 shown in FIG. 9 except a feature that the positions of the primer-immobilized regions 11 and the probe-immobilized regions 13 are on side surfaces of the channel 15.

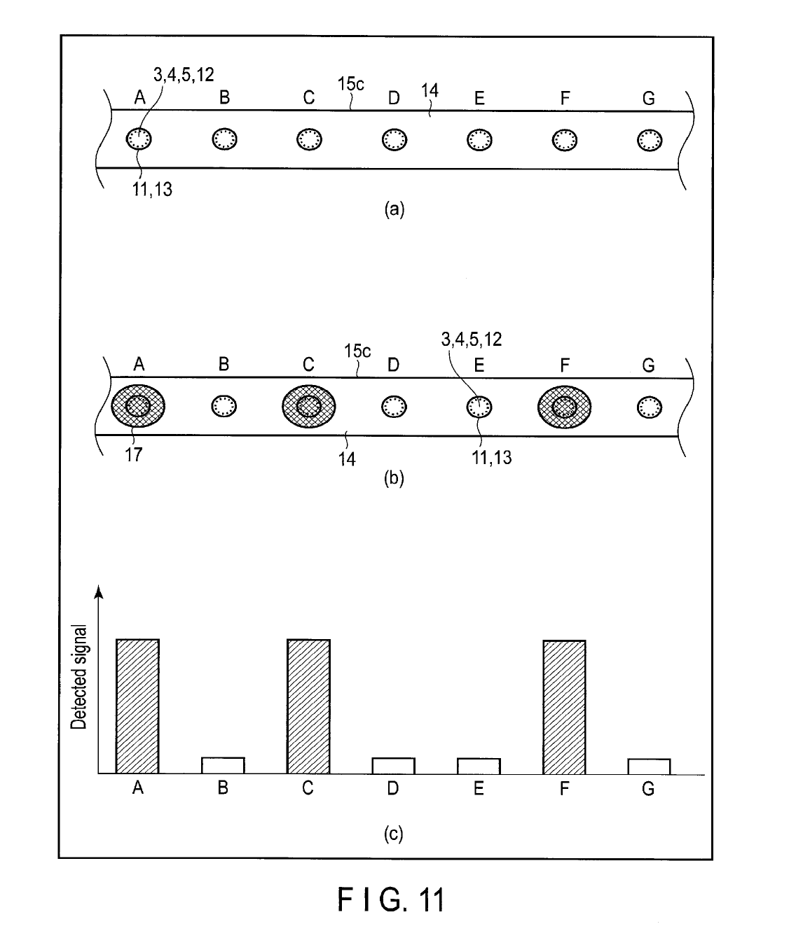

FIG. 11 shows a channel 15 in a nucleic acid probe immobilized substrate similar to the array-type nucleic acid probe immobilized substrate 1 shown in FIG. 9 except a feature that the primer-immobilized regions 11 and the probe-immobilized regions 13 are arranged on substantially the same positions.

Each of FIG. 9(a), FIG. 10(a) and FIG. 11(a) shows a state in which the primer sets 12 and the nucleic acid probe sets are immobilized in the channel 15. In any one of FIG. 9(a), FIG. 10(a) and FIG. 11(a), regions A, B, C, D, E, F, and G are arranged on an inner surface of the channel 15 extended along the longitudinal direction of the channel 15. In each of the regions A, B, C, D, E, F, and G, the primer sets 12 are immobilized to be releasable, and the nucleic acid probes 3 to be arranged to correspond thereto are immobilized. The primer sets 12 immobilized in the respective regions A to G are designed to be different from each other so as to amplify mutually different target sequences. The covering nucleic acid chains 5 hybridized to the nucleic acid probes 3 have mutually different sequences to detect first sequences different in region. In other words, the primer sets 12 and the nucleic acid probes 3 having the sequences of different types in region as the target sequences and the first sequences are immobilized in the primer-immobilized regions 11 and the probe-immobilized regions 13 arranged in the respective regions A to G, and the nucleic acid probes 3 are hybridized to the covering nucleic acid chains 5.

More specifically, the primer sets 12 and the nucleic acid probes 3 are immobilized at positions explained below, in the regions A to G in each of the channels. In FIG. 9(a), the primer sets 12 and the nucleic acid probes 3 corresponding thereto are arranged adjacent to each other, along a longitudinal direction of the channel 15, on the bottom surface 16 of the channel 15 corresponding to positions of respective regions. In FIG. 10(a), the primer sets 12 are releasably immobilized to one of side surfaces of the channel 15 corresponding to positions of respective regions. The nucleic acid probes 3 are immobilized to the other side surface of the channel 15 opposed to the side surface to which the primer set 12 is immobilized. In FIG. 11(a), the primer sets 12 are releasably immobilized and the nucleic acid probes 3 are immobilized, to the same positions of the bottom surface 16 corresponding to positions of the respective regions of the channel 15. The covering nucleic acid chains 5 are bonded to the nucleic acid probes 3.

FIG. 9(b), FIG. 10(b) and FIG. 11(b) show states in which a reaction solution has been added to the respective channels 15 shown in FIG. 9(a), FIG. 10(a) and FIG. 11(a). When the reaction solution is added to each channel 15, the primer sets 12 are released and diffused in the reaction solution. If a template nucleic acid which is to be a target is present in the reaction solution, the amplification reaction occurs and the amplification product 6 is produced. Each of FIG. 9(b), FIG. 10(b) and FIG. 11(b) schematically illustrates a region in which the amplification reaction occurs and proceeds as an amplification region 17. If the first sequence that should be detected is included in the amplification product 6 produced in the amplification region 17, the nucleic acid probes 3 and the amplification product 6 make a competitive reaction for the corresponding covering nucleic acid chains 5. Then, the hybridization between the nucleic acid probes 3 and the covering nucleic acid chains 5 is eliminated and a detection signal is produced.

FIG. 9(c), FIG. 10(c) and FIG. 11(c) illustrate graphs representing magnitudes of detection signals detected in regions A to G of the respective channels 15a, b, c. Each of the graphs indicates that a sample contained in the added reaction solution includes the target sequences amplified by the primer sets 12 immobilized in the regions A, C and F of each channel 15a, b, c and that amplification products 6 obtained by the primer sets 12 include the first sequence complementary to the sequences of the first sequence bonding region 8′ of the covering nucleic acid chains 5 hybridizing to the nucleic acid probes 3 immobilized to the regions A, C and F. In other words, in the graphs of FIG. 9(c), FIG. 10(c) and FIG. 11(c), the signals obtained in relation to the regions A, C and F (represented as “detection signals” in the drawings) are detection signals greater than the background level. Such a result indicates that the amplification products of interest including the first sequence are present in the sample. The signals obtained in relation to the regions B, D and E are below the background level. It is discriminated that “the target nucleic acid to be detected is present in the sample”, based on these results.

In the above-described example, the primer sets 12 alone are immobilized to the substrate 2 as reagents for amplification. However, the primer sets are not limited to this example, and other components necessary for amplification, for example, enzymes such as polymerase and a reverse transcriptase, a substrate substance, a substrate and/or a buffer, can be immobilized on the substrate 2 together with the primer sets 12, under conditions that the primer sets 12 are immobilized in each immobilization region for each type. In this case, substances to be fixed may be included in a desired liquid medium together with the primer sets 12, and immobilized by dropping and drying, similarly to the above-explained method. When the amplification reaction is carried out in the nucleic acid probe immobilized substrate 1, a composition of the reaction solution to be added thereto may be selected according to fixed components.

In one of the embodiments, the nucleic acid probe immobilized substrate may be a nucleic acid probe immobilized substrate for detecting first to n-th target nucleic acids (n is an integer greater than or equal to 2). The first to n-th target nucleic acids include eleventh to 1n-th sequences and/or first to n-th complementary sequences which are the complementary sequences thereof, respectively. The nucleic acid probe immobilized substrate may comprise:

(i) a substrate configured such that an isothermal amplification reaction using first to n-th primer sets supports a reaction field for producing first to n-th amplification products including the 1₁-th to mutually different 1ₙ-th sequences, respectively, with the first to n-th target nucleic acids used as templates;

(ii) first to n-th probe immobilized regions arranged independently on at least one surface of the substrate in contact with the reaction field when the reaction field is formed;

(iii) first to n-th nucleic acid probes including first to n-th nucleic acid chains which include 2₁-th to 2ₙ-th sequences immobilized to the respective first to n-th probe immobilized regions, and first to n-th labeling substances which produce detectable signals bonded to the respective first to n-th nucleic acid chains, respectively; and

(iv) first to n-th covering nucleic acid chains which include 1₁-th to 1ₙ-th sequence bonding regions complementary to the respective 1₁-th to 1ₙ-th sequences, and 2₁-th to 2ₙ-th sequence bonding regions complementary to the respective 2₁-th to 2ₙ-th sequences, and which are bonded to the respective first to n-th nucleic acid probes by hybridization to the respective 2₁-th to 2ₙ-th sequences in the 2₁-th to 2ₙ-th sequence bonding regions, thereby inhibiting detectabilities of the signals from the respective first to n-th nucleic acid probes.

Base sequences of the respective first to n-th nucleic acid probes and the respective first to n-th covering nucleic acid chains are designed to obtain competition between the corresponding first to n-th amplification products and the first to n-th nucleic acid probe sequences, for the first to n-th covering nucleic acid chains, desorption of the first to n-th covering nucleic acid chains from the first to n-th nucleic acid probes, caused by the competition, and bonding of the first to n-th covering nucleic acid chains to the first to n-th amplification products via hybridization to the 1₁-th to 1ₙ-th sequences in the respective 1₁-th to 1ₙ-th sequence bonding regions, under the isothermal amplification reaction conditions in the formed reaction field.

The bonding of the first to n-th covering nucleic acid chains to the first to n-th amplification products to the respectively corresponding first to n-th nucleic acid probes by the hybridization, under the isothermal amplification reaction conditions in the formed reaction field, in such an embodiment, will be explained below. The bonding is maintained if the nucleic acids including the corresponding 1₁-th to 1ₙ-th sequences, respectively, are not present in the reaction field. The bonding is eliminated if the nucleic acids including the corresponding 1₁-th to 1ₙ-th sequences, respectively, are present in the reaction field and if the nucleic acids and the corresponding nucleic acid probes are competitive to the corresponding covering nucleic acid chains. Such conditions can be met by, for example, adjusting lengths and Tm values of base sequences in the first to n-th nucleic acid probes and the first to n-th covering nucleic acid chains.

The nucleic acid probe immobilized substrate may further comprise first to n-th primer-immobilized regions arranged at the same positions as or independently in close vicinity of the respective first to n-th probe-immobilized regions, and the first to n-th primer sets releasably immobilized to the respective first to n-th primer-immobilized regions.

It is preferable that the 2₁-th to 2ₙ-th sequences have the same sequences as each other and the eleventh to 1n-th sequence bonding regions have mutually different sequences.

In prior art, the nucleic acid chain to be detected was hybridized to the nucleic acid probe not with the covering nucleic acid chain 5, but directly, and then the nucleic acid chain is detected. According to this technology, the amplification and the detection can hardly be executed simultaneously in one reaction field. For example, a salt concentration suitable to the amplification reaction is a salt concentration suitable to hybridization of nucleic acid. For this reason, a stable hybridization could not be obtained in the reaction field in which the amplification reaction was executed, and the amplification reaction and the measurement of hybridization of the nucleic acid could hardly be conducted in one reaction field.

According to the nucleic acid probe immobilized substrate of the present embodiment, the detection is executed by using elimination of the hybridization between the nucleic acid probes and the covering nucleic acid chains, which occurs in response to the presence of the amplification product. For this reason, the amplification product can be measured with high sensitivity and high accuracy, simultaneously with the amplification reaction, under the amplification reaction conditions. The target nucleic acids in the sample can be thereby quantified. In addition, since a plurality of target nucleic acids can be amplified simultaneously and detected simultaneously, a test on the target nucleic acids can be executed for a time shorter than that in prior art. An error is unlikely to occur in selection of the sample.

When a reaction tool comprising a channel is used, the reaction can be made in the channel. The array-type nucleic acid probe immobilized substrate having more excellent operability can be therefore provided.

In addition, the reaction solution can be extended to all the primer sets and the nucleic acid probes more easily, in a short time, by providing the channel. Detection of the target nucleic acid can also be executed for each of a plurality of samples, by providing a plurality of channels.

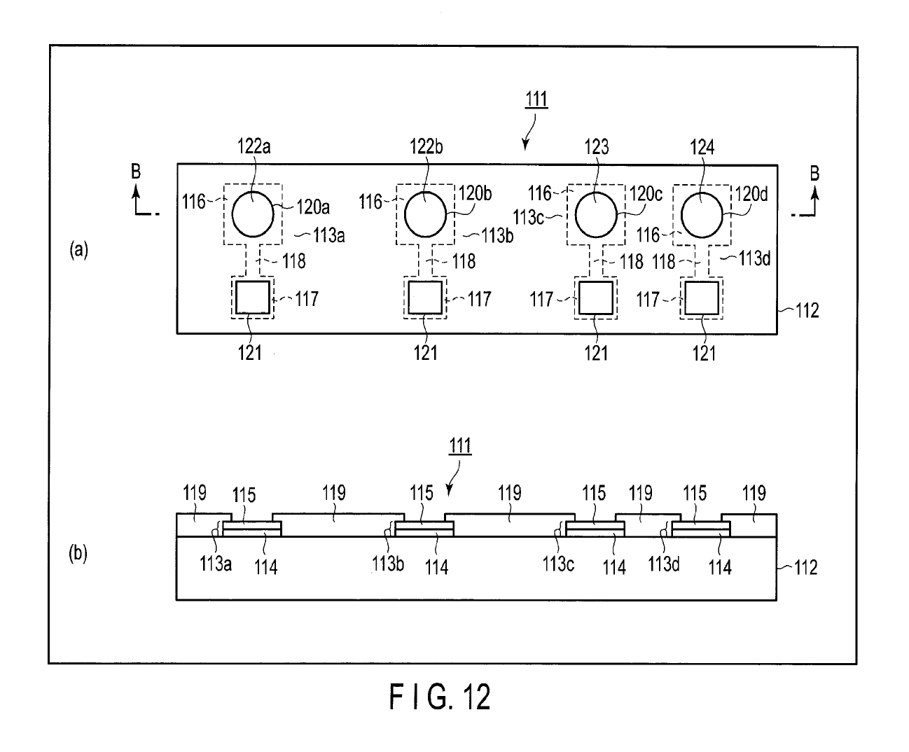

An example of a multi-nucleic-acid amplification detection reaction tool for detecting a plurality of target nucleic acid will be explained with reference to FIGS. 12 to 15 as another embodiment.

(1) Chip Material

First, an example of a configuration of a chip material of a multi-nucleic-acid amplification detection reaction tool configured to detect a signal from a nucleic acid probe by electrochemical detection and a method of producing the chip material will be explained with reference to FIG. 12(a) and FIG. 12(b). FIG. 12(a) is a plan view of a chip material 111, and FIG. 12(b) is a cross-sectional view of the chip material 111 seen along line B-B in FIG. 11(a).

The chip material 111 includes, on a rectangular base plate 112, for example, four electrodes 113a to 113d arranged along a longitudinal direction of the base plate. Each of the electrodes 113a to 113d has a structure in which a first metal thin-film pattern 114 and a second metal thin-film pattern 115 are stacked in this order. Each of the electrodes 113a to 113d has a shape in which a large rectangular portion 116 and a small rectangular portion 117 are connected to each other by a fine wire 118. An insulating film 119 is coated on the base plate 112 including each of the electrodes 113a to 113d. A circular window 120 is opened at a part of the insulating film 119 corresponding to the large rectangular portion 116. A rectangular window 121 is opened at a part of the insulating film 119 corresponding to the small rectangular portion 117. The large rectangular portion 116 exposed from the circular window 120 of the electrode 113a functions as a first working electrode 122a. The large rectangular portion 116 exposed from the circular window 120 of the electrode 113b functions as a second working electrode 122b. The large rectangular portion 116 exposed from the circular window 120 of the electrode 113c functions as a counter-electrode 123. The large rectangular portion 116 exposed from the circular window 120 of the electrode 113d functions as a reference electrode 124. The small rectangular portion 117 exposed from the rectangular window 121 of each of the electrodes 113a to 113d functions a prober contact portion.

The chip material 111 can be prepared by the following method.

First, a first metal thin film and a second metal thin film are stacked in this order on the base plate 112 by, for example, sputtering or vacuum deposition. Subsequently, for example, four electrodes 113a to 113d are formed along the longitudinal direction of the base plate 112 by sequentially subjecting the metal thin films to selective etching using, for example, a resist pattern as a mask, and by stacking the first metal thin-film pattern 114 and the second metal thin-film pattern 115 in this order. Each of the electrodes 113a to 113d has a shape in which the large rectangular portion 116 and the small rectangular portion 117 are connected to each other by a fine wire 118.

Then, the insulating film 119 is deposited on the base plate 112 including the electrodes 113a to 113d by, for example, sputtering or CVD. Subsequently, a part of the insulating film 119 corresponding to the large rectangular portion 116 of each of the electrodes 113a to 113d and a part of the insulating film 119 corresponding to the small rectangular portion 117 of each of the electrodes 113a to 113d are selectively etched to open the circular window 120 at the part of the insulating film 119 corresponding to the large rectangular portion 116 and the rectangular window 121 at the part of the insulating film 119 corresponding to the small rectangular portion 117. The above-explained chip material 111 is thereby prepared.

The base plate 112 is formed from a glass such as Pyrex (trademark) (registered trademark) or a resin.

The first metal thin film acts as a base metal film which brings the second metal thin film into close contact with the base plate 112, and is of, for example, Ti. The second metal thin film is of, for example, Au.

Examples of etching for patterning the first and second metal thin films include plasma etching or reactive ion etching using an etching gas.

Examples of the insulating film 119 include metal oxide films such as a silicon oxide film and metal nitride films such as a silicon nitride film.

Examples of etching for patterning the insulating film 119 include plasma etching or reactive ion etching using an etching gas.

(2) Multi-Nucleic-Acid Amplification Detection Reaction Tool

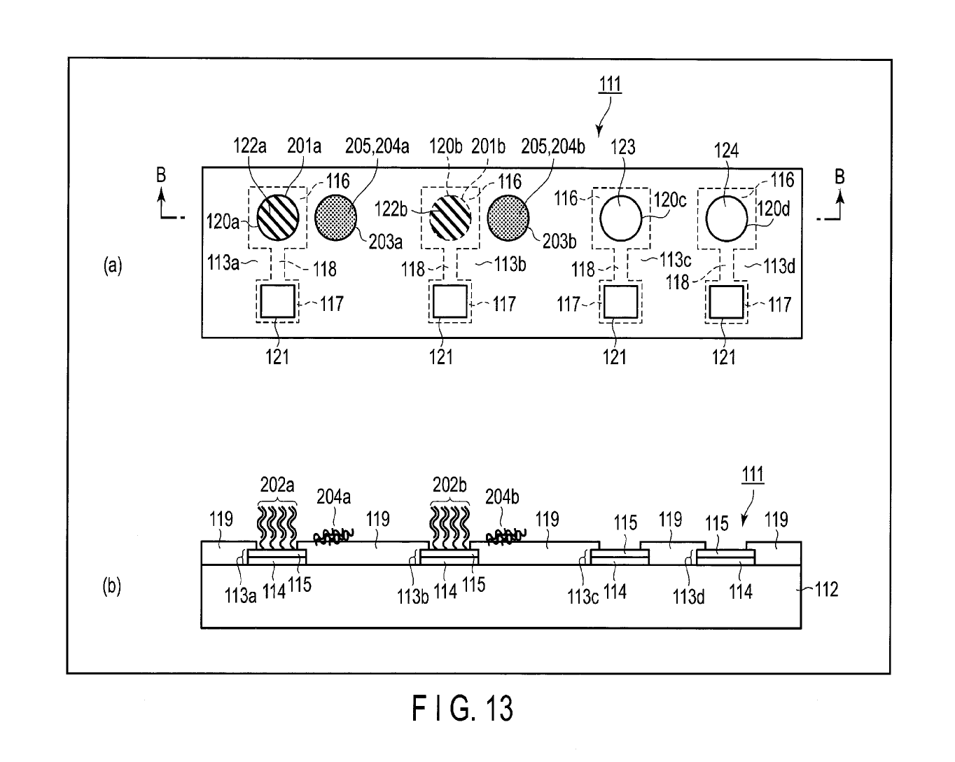

Next, an example of a configuration of a multi-nucleic-acid amplification detection reaction tool with primer sets and probe nucleic acids immobilized on the chip material 111 produced as explained above in (1) and a method of producing the multi-nucleic-acid amplification detection reaction tool will be explained with reference to FIG. 13(a) and FIG. 13(b). FIG. 13(a) is a plan view of the multi-nucleic-acid amplification detection reaction tool, and FIG. 13(b) is a cross-sectional view of the multi-nucleic-acid amplification detection reaction tool seen along line B-B in FIG. 13(a). The multi-nucleic-acid amplification detection reaction tool is a device for detecting presence of two types of amplification products each including two mutually different sequences. Each of two types of target nucleic acids that should be detected includes the 1₁-th sequence and the 1₂-th sequence. A first nucleic acid probe 202a for detection of the 1₁-th sequence contains a first nucleic acid chain including the 1₁-th sequence and a first labeling substance bonded to the first nucleic acid chain. This forms a first double-stranded nucleic acid which bonds to and contains a first covering nucleic acid chain having a sequence complementary to the sequence of the first nucleic acid chain, in an initial state. A first nucleic acid probe 202b for detection of the 1₂-th sequence contains a second nucleic acid chain including the 1₂-th sequence and a second labeling substance bonded to the second nucleic acid chain. This forms a second double-stranded nucleic acid which contains a second covering nucleic acid chain having a sequence complementary to the sequence of the second nucleic acid chain, in an initial state.

The first working electrode 122a of the electrode 113a formed on the chip material 111 is defined as a first probe-immobilized region 201a, and the first double-stranded nucleic acid is immobilized to the first probe-immobilized region 201a. A plurality of first double-stranded nucleic acids each containing the first nucleic acid probe 202a are immobilized to one immobilization region, and function as one nucleic acid probe group.

Similarly, the second working electrode 122b of the electrode 113b is defined as a second probe-immobilized region, and a plurality of second double-stranded nucleic acids each containing a second nucleic acid probe 202b are immobilized to the second probe-immobilized region. A plurality of second double-stranded nucleic acids each containing the second nucleic acid probe 202b are immobilized to one immobilization region, and function as one nucleic acid probe group.

Examples of a method of immobilizing the first nucleic acid probe 202a and the second nucleic acid probe 202b to the first probe-immobilized region 201a and the second probe-immobilized region 201b include a method introducing a thiol group to a 3′ terminal of each of the first nucleic acid probe 202a and the second nucleic acid probe 202b in a case of using the chip material 111 comprising metallic electrodes.

Then, the first primer-immobilized region 203a is arranged in close vicinity of the first working electrode 122a, and the second primer-immobilized region 203b is arranged in close vicinity of the second working electrode 122b. A first primer set 204a and a thickener 205 are releasably immobilized on the first primer-immobilized region 203a, and a second primer set 204b and a thickener 205 are releasably immobilized on the second primer-immobilized region 203b. The multi-nucleic-acid amplification detection reaction tool is thereby prepared.

The first primer set 204a contains a plurality of primers designed to amplify the 1₁-th sequence, and the second primer set 204b contains a plurality of primers designed to amplify the 1₂-th sequence different from the 1₁-th sequence.

Immobilizing the first primer set 204a and the second primer set 204b to the first primer-immobilized region 203a and the second primer-immobilized region 203b, respectively, is executed by, for example, including the primer sets in a liquid such as water, a buffer solution or an organic solvent, dropping the liquid on the regions, and leaving the liquid for a time until the liquid is dried under an appropriate temperature condition such as room temperature, for example, ten minutes at room temperature.

The thickener may be used arbitrarily and, if the thickener is used, the thickener may be immobilized or may be contained in the reaction solution. Immobilization of the thickener is executed by dissolving a desired thickener in a liquid, and dropping and drying the liquid at a desired position before or after the immobilization of the primer sets. The liquid in which the thickener is dissolved may be a liquid prepared for the immobilization of the primer sets or the other liquid. The immobilization may be executed in the primer-immobilized region or in close vicinity of the primer-immobilized region and/or the probe-immobilized region.

(3) Multi-Nucleic-Acid Amplification Detection Reaction Tool in Use

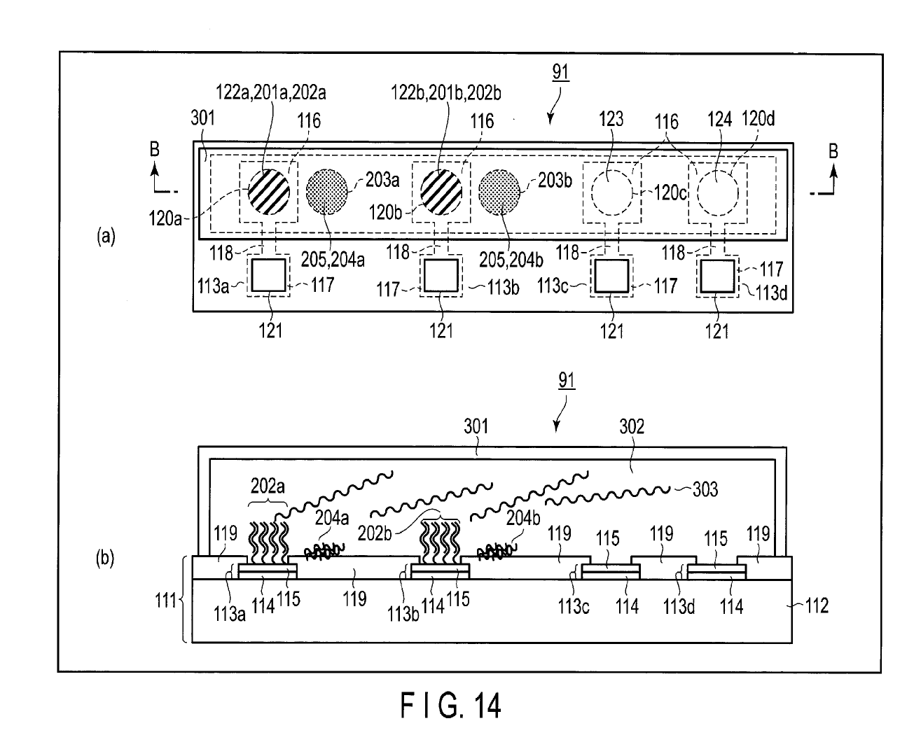

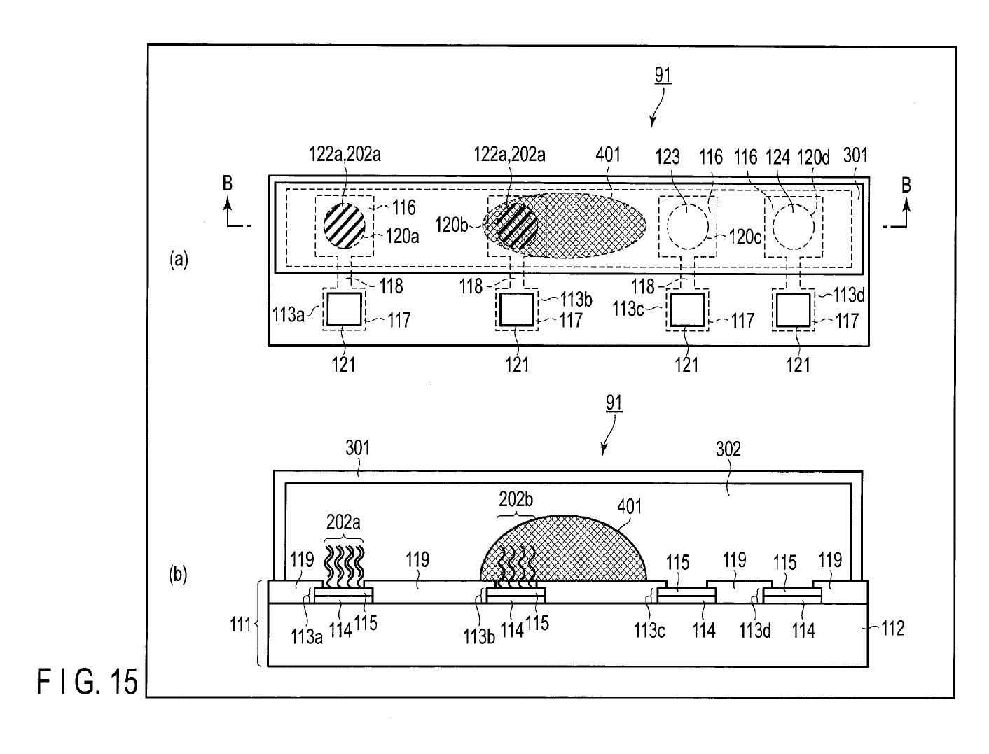

An example of use of the multi-nucleic-acid amplification detection reaction tool prepared as explained above in (2) will be explained with reference to FIG. 14 and FIG. 15.

FIG. 14(a) is a plan view of the multi-nucleic-acid amplification detection reaction tool in use, and FIG. 14(b) is a cross-sectional view of the multi-nucleic-acid amplification detection reaction tool seen along line B-B in FIG. 14(a).

When a multi-nucleic-acid amplification detection reaction tool 91 of the present embodiment is used, the reaction solution is maintained such that the first working electrode 122a, the second working electrode 122b, the counter-electrode 123 and the reference electrode 124 formed in the electrodes 113a to 113d, respectively, and the first primer-immobilized region 203a and the second primer-immobilized region 203b are included in the same single reaction field. For this purpose, a cover 301 formed by molding, for example, a silicon resin such as a silicon rubber and/or a fluororesin, etc., or any resin by any resin molding method that is publicly known itself, such as, for example, extrusion molding, injection molding or stamping molding and/or bonding using an adhesive is mounted on the multi-nucleic-acid amplification detection reaction tool 91 before the multi-nucleic-acid amplification detection reaction tool 91 is used. After the cover 301 is mounted, a reaction solution 302 containing a template nucleic acid 303 is added to a space formed by the multi-nucleic-acid amplification detection reaction tool 91 and the cover 301.

In the multi-nucleic-acid amplification detection reaction tool 91 on which the cover 301 is mounted, the small rectangular portion 117 exposed from the rectangular window 121 of each of the electrodes 113a to 113d is exposed.

Examples of mounting the cover 301 on the multi-nucleic-acid amplification detection reaction tool 91 include, for example, press bonding, bonding using an adhesive, etc.

Then, the reaction solution 302 is added after the cover 301 is mounted on the multi-nucleic-acid amplification detection reaction tool 91.

As a method of adding a liquid to a space formed by the multi-nucleic-acid amplification detection reaction tool 91 and the cover 301, for example, an opening may be preliminarily formed at a part of the cover 301 to add the liquid through the opening, or a syringe having a sharp tip such as a needle-like tip may be inserted into a part of the cover 301 to add the liquid.

The reaction solution 302 may include, for example, the sample, the thickener, the amplification reagent, an enzyme such as polymerase, a substrate such as deoxynucleoside triphosphate necessary for formation of a new polynucleotide chain using a primer as a start point and, when performing reverse transcription in parallel, a reverse transcriptase and a substrate necessary therefor, etc., and a buffer such as a salt for maintaining a proper amplification environment, etc. When a template nucleic acid including a target sequence to be amplified by a primer set immobilized on a specific primer-immobilized region is present in a sample to be examined, an amplification product is formed in the reaction field including the primer-immobilized region and a probe-immobilized region corresponding thereto. This situation is schematically illustrated in FIG. 15.

FIG. 15(a) schematically illustrates a state in which an amplification product is formed in a reaction field 401. FIG. 15(a) is a plan view of the multi-nucleic-acid amplification detection reaction tool in use, and FIG. 15(b) is a cross-sectional view of the multi-nucleic-acid amplification detection reaction tool seen along line B-B in FIG. 15(a). Since a nucleic acid including a sequence to which the second primer set 204b can be bonded is included in the added sample in FIG. 14 as explained above, the second primer set is released and diffused to the reaction field 401, and encounters a template nucleic acid, and an amplification reaction occurs as illustrated in FIG. 15(a) and FIG. 15(b). The amplification product is thereby formed. The amplification product of the second primer set 204b is diffused to the periphery of the second primer-immobilized region 203b and reaches the second probe-immobilized region 201b. When the arriving amplification product includes the twelfth sequence, the amplification product and the covering nucleic acid chain bonded to the second nucleic acid probe 202b become competitive, the covering nucleic acid chain is desorbed from the second nucleic acid probe 202b and is hybridized to the amplification product. The nucleic acid probe 202b thereby becomes a single-stranded nucleic acid. Since the nucleic acid probe 202b becomes a single-stranded nucleic acid, the signal from the labeling substance included in the second nucleic acid probe can be detected.

The signal from the nucleic acid probe can be produced by, for example, bringing a prober into contact with the small rectangular portion 117 exposed from the rectangular window 121 of each of the electrodes 113a to 113d, and measuring a current response of the labeling substance.

By using an array-type primer probe chip using electrochemical detection, a target nucleic acid included in the sample can be amplified more easily, in a short time, and then the nucleic acid which should be detected, included in the amplification product, can be detected simply and more accurately. A plurality of target nucleic acids can also be detected quantitatively.

(4) Method of Detecting Nucleic Acid

A method of amplifying a plurality of target nucleic acids and detecting an amplification product with a signal from a labeling substance used as an indicator by using, for example, the above-explained multi-nucleic-acid amplification detection reaction tool is also provided as another embodiment.

A method of detecting a target nucleic acid, comprising a step of releasably immobilizing plural types of primer sets designed to amplify plural types of target nucleic acids, respectively, to at least one surface of a support such as a specific container, a tube, a dish, or a base plate provided with a channel, and/or a step of immobilizing at least one type of nucleic acid probe to a probe-immobilized region, is also provided as yet another embodiment.

Such target nucleic acid detection methods may comprise, for example, releasably immobilizing plural types of primer sets designed to amplify plural types of target nucleic acids, respectively, to at least one surface of a desired substrate, immobilizing nucleic acid probes corresponding to the respective primer sets at positions at which a plurality of primers are immobilized or positions near the positions, adding a reaction solution to the primer sets and the nucleic acid probes, adding a sample to the reaction solution, forming a reaction field with the reaction solution, maintaining a reaction environment of the reaction field as an environment suitable for an amplification reaction, executing a nucleic acid amplification reaction, and detecting and/or measuring detectable signals from the nucleic acid probes.

The reaction solution may include a reagent necessary for the amplification reaction, for example, an enzyme such as polymerase, a substrate such as deoxynucleoside triphosphate necessary for formation of a new polynucleotide chain using the primer as a start point and, when performing reverse transcription in parallel, an enzyme such as a reverse transcriptase and a substrate necessary therefor, etc., and a buffer such as a salt for maintaining a proper amplification environment, etc. A thickener may be further included as a reaction reagent.

The addition of the sample to the reaction solution may be executed before or after the addition of the reaction solution to the primer sets and the nucleic acid probes.

The formation and maintenance of the reaction environment may be executed by adjusting a temperature and/or a salt concentration within a range suitable for the amplification reaction. The nucleic acid amplification reaction executed in the reaction environment is an amplification reaction of the target nucleic acid corresponding to the plural types of primer sets, and may be executed sequentially or in parallel with the plurality of primer sets. According to the method of the embodiment, the amplification reaction is executed with the plurality of primer sets in a sequential space of one reaction tool, but such an amplification reaction may be a reaction which is generally called a multi-nucleic-acid amplification reaction.

According to the method of the embodiment, the target nucleic acid can be detected simply, at high sensitivity. In addition, plural types of target sequences can be amplified independently in parallel without interference from different sequences. The presence and absence and/or the amount of the amplification product including a specific sequence generated by the amplification reaction can be detected under isothermal amplification reaction conditions, in parallel with the amplification reaction. Furthermore, if the thickener is applied, amplification reactions which are carried out in parallel for a plurality of types of target sequences can be achieved more efficiently.

The thickener may be immobilized to the substrate as explained above, instead of adding the thickener to the reaction solution. Alternatively, the thickener may be contained in the reaction solution and provided to the reaction field and/or may be immobilized to the support surface and provided.

If the amplification reaction is carried out by using the multi-nucleic-acid amplification reaction tool, the amplification reaction proceeds near the region in which the primer sets are immobilized alone, and detection of the amplification product is executed in parallel with the amplification reaction, and a plurality of amplification reactions of various types of target nucleic acids can be made to proceed independently without interfering with one another although the amplification reactions are carried out in the same container and/or the same reaction solution. According to the embodiment, the amplification product can be executed at high sensitivity and detected quantitatively. The target nucleic acid can be detected quantitatively, at high sensitivity.

3-2. Target Nucleic Acid Measurement Method

A target nucleic acid measurement method will be provided as one of the embodiments. The target nucleic acid measurement method may comprise preparing a nucleic acid probe immobilized substrate, bringing a sample containing a target nucleic acid to a reaction field, amplifying the target nucleic acid, reacting an amplification product with the nucleic acid probe immobilized substrate to eliminate hybridization between a nucleic acid probe and a covering nucleic acid chain, and detecting variation of a detectable signal emitted from a labeling substance by elimination of the hybridization.

For example, the target nucleic acid measurement method may comprise detecting a target nucleic acid containing a first sequence and/or a complementary sequence thereof. The method may comprise the following steps:

(a) making isothermal amplification using a primer set for formation of an amplification product including a first sequence using a target nucleic acid as a template, under presence of a nucleic acid probe in which signal detectability is inhibited, by bonding a covering nucleic acid chain including a first sequence bonding region complementary to the first sequence and a second sequence bonding region complementary to a second sequence, to a nucleic acid probe by hybridization with the second sequence in the second sequence bonding region, the nucleic acid probe including a labeling substance which produces a detectable signal and being immobilized to a solid phase by a nucleic acid chain including the second sequence different from the first sequence;

(b) making the amplification product formed in a reaction of the isothermal amplification competitive with the nucleic acid probe, desorbing the covering nucleic acid chain from the nucleic acid probe, and bonding the amplification product to the covering nucleic acid chain by hybridization of the first sequence in the first sequence bonding region; and

(c) monitoring the signal from the nucleic acid probe or detecting the signal at least two times, under a conditions for the reaction of the isothermal amplification.

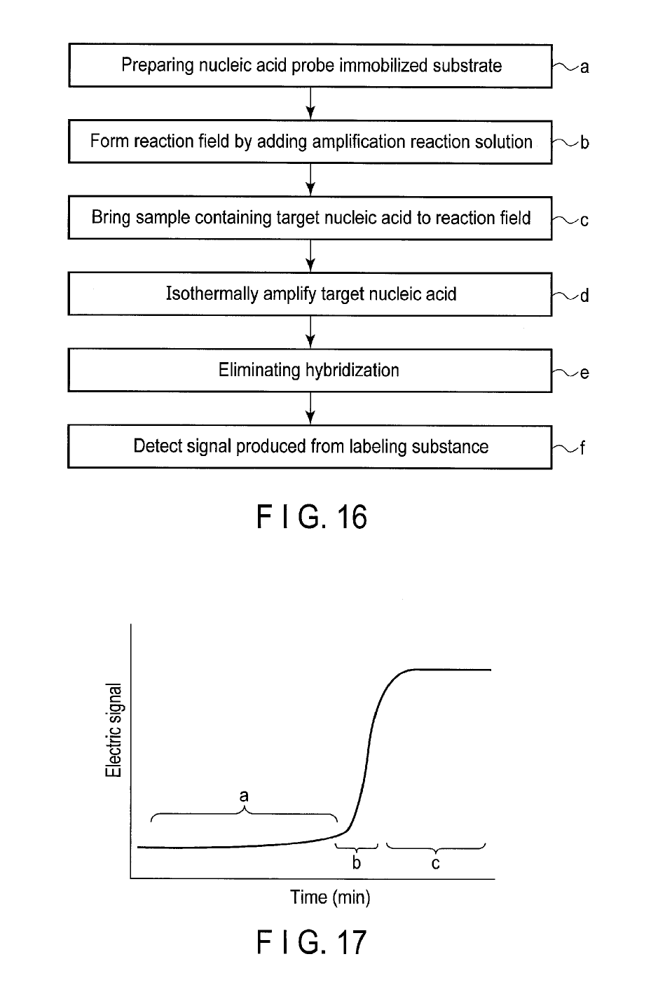

The target nucleic acid detection method may be executed with the nucleic acid probe immobilized substrate as shown in FIG. 16. The method may comprise (a) preparing a nucleic acid probe immobilized substrate, (b) adding an amplification reaction solution to form a reaction field, (c) bringing a target nucleic acid to the reaction field, (d) isothermally amplifying the target nucleic acid, (e) eliminating hybridization between a nucleic acid probe and a covering nucleic acid chain by making the amplification product and the nucleic acid probe competitive, and (f) detecting a signal from a labeling substance.

The preparation of the nucleic acid probe immobilized substrate may comprise immobilizing the nucleic acid probe on at least one surface which is in contact with the reaction field when the reaction field is formed, of a substrate configured to support the reaction field, hybridizing the nucleic acid probe with the covering nucleic acid chain, and bonding the labeling substance which generates the detectable signal to the nucleic acid probe.

As explained above, the nucleic acid probe immobilized substrate may immobilize the primer set releasably in the primer-immobilized region and include the primer set.

The addition of the sample to the inside of the substrate may be executed by, for example, preliminarily adding the sample to the reaction solution prior to adding the reaction solution to the nucleic acid probe immobilized substrate. Alternatively, it may be executed by adding the reaction solution to the nucleic acid probe immobilized substrate and then adding the sample to the reaction solution. It may be executed by adding the sample to the nucleic acid probe immobilized substrate prior to adding the reaction solution to the nucleic acid probe immobilized substrate.

The reaction solution may be a liquid phase such that after the immobilized fixed primer set is separated, an amplification reaction between the primer set and the target nucleic acid can occur. This reaction solution may be injected mechanically or manually to the reaction field (initially filled with air), in any method, prior to the start of the amplification reaction.

If a threshold value is set in advance and a time required for the detection signal to exceed the threshold value is measured as a rise time, quantification of the target nucleic acid can be executed based on the obtained result. Alternatively, the quantification of the target nucleic acid may also be executed by preparing a plurality of different standard sample nucleic acids for which abundance of the nucleic acids is known, measuring using the standard sample nucleic acids, forming a calibration curve based on a measurement result obtained for the abundance of each nucleic acid and comparing the measurement result of the target nucleic acid with the formed calibration curve, and calculating the abundance of the target nucleic acid in the sample.

According to the method of the embodiment, the target nucleic acid can be quantified simply. In addition, more target nucleic acids can be tested in a time shorter than that in prior art. An error may hardly occur in selection of the sample.

### 4. Third Embodiment

Another example of the method for detecting the target nucleic acid of the above-described first embodiment, in which each of a sequence of the nucleic acid probe and the covering nucleic acid chain are the sequences of the above (a) is described as the third embodiment with reference to FIG. 17.

In the second embodiment, an example of the method for detecting the target nucleic acid in which a labeling substance is bonded to a nucleic acid probe is described. However, the labeling substance that emits a signal may not be bonded to the nucleic acid chain but may be contained in a reaction solution. The third embodiment is an example of such a method.

An example of the probe immobilized substrate used for the third embodiment is shown in FIG. 17. This example has the same constitution as those of FIGS. 4(a), (b), and (c) except that the labeling substance is present in the reaction solution and is not bonded to the nucleic acid probe. A substrate 2 of a probe immobilized substrate 101 comprises an electrode 2a on a surface of at least one part of the substrate 2 forming a reaction field. The nucleic acid probe 3 is immobilized to the electrode 2a (FIG. 17(a)). The covering nucleic acid chain 5 is bonded to the nucleic acid probe 3. The labeling substance 44 is present in the reaction solution containing them. The labeling substance 44 is a substance which generates an electrochemical signal and is a substance in which detection of a signal is inhibited by elongation of a covering nucleic acid chain using an amplification product as a template. In addition, the labeling substance may also be an electrochemically active substance having a negative charge in the reaction solution, i.e., in the reaction field. A detectable signal from the labeling substance 44 is an electric signal, which is detected by an electrode to which the nucleic acid probe is immobilized. In addition, the detection of the detectable signal produced by the labeling substance 44 is inhibited by a presence or an increase in abundance of the nucleic acid which is bonded to the nucleic acid probe. That is, as shown in FIG. 17A, since the nucleic acid probe 3 and the covering nucleic acid chain 5 bonded thereto are present above the electrode 2a, negative charges derived from the nucleic acid are largely present as compared to other regions. As a result, the detection of the electric signal from the labeling substance 44 is inhibited. When the reaction field is maintained under the isothermal amplification condition, an amplification reaction using a target nucleic acid as a template proceeds, such that an amplification product 6 is formed (FIG. 17(b)), and an abundance is increased over time. The amplification product 6 competes with the nucleic acid probe 3, and the covering nucleic acid chain 5 is desorbed from the nucleic acid probe 3. As a result, the nucleic acid bonded to the electrode becomes a single strand from a double strand (FIG. 17(c)). When the nucleic acid probe 3 becomes the single strand, negative charges are decreased as compared to a case where it is present in the double strand. As a result, a larger electric signal is generated when it is present in the double strand. By detecting a difference of the electric signals, it is possible to detect the presence of the target nucleic acid or to measure abundance thereof, for example, concentration.

The labeling substance used in the third embodiment may be an electrochemically active substance having a negative or positive charge in the reaction field among the substances in which signal detection is inhibited by elongation of the covering nucleic acid chain using the amplification product as the template. An example of such a substance may be an oxidizing agent or the like in which a redox potential thereof is capable of being a detectable electrical chemical signal. Examples of the labeling substance include, for example, ferricyanide ion, ferrocyanide ion, iron complex ion, ruthenium complex ion, cobalt complex ion, etc. These labeling substances are obtained by dissolving potassium ferricyanide, potassium ferrocyanide, iron complex, ruthenium complex and cobalt complex in the reaction solution. The concentration of such labeling substance in the reaction solution may be, for example, 10 μM to 100 mM, and may also be, for example, about 1 mM.

For example, when a ferricyanide ion (Fe(CN)₆⁴⁻) is used as the labeling substance, electrons are released by an oxidation reaction in which Fe(CN)₆⁴⁻ is converted into Fe(CN)₆³⁻. These electrons flow to the electrode when Fe(CN)₆⁴⁻ is close to the electrode. This flow of electrons produces the electrochemical signal to be detected.

These labeling substances may be used in combination with other labeling substances. For example, when an electrochemically active substance having a negative or positive charge in the reaction field is combined with a nucleic acid probe labeled with ferrocene, since the ferrocene acts as a mediator to amplify the electrochemical signal, sensitivity may be further improved.

When the electrochemically active substance having a negative charge in such a reaction field is used as the labeling substance, it is kept away from a portion in the reaction solution in which a plurality of relatively long nucleic acid chains or a plurality of relatively short nucleic acids are present. This is because the nucleic acid chain has the negative charge likewise, and the charge of the nucleic acid repels the labeling substance. By such a property, detection of the signal from the labeling substance, for example, a redox potential, through the nucleic acid probe to which the covering nucleic acid chain is bonded is inhibited.

On the other hand, as the nucleic acid including a first sequence 8 such as the amplification product 6, etc., is amplified in the reaction field to increase the nucleic acid probe of one chain (FIG. 17(b)), an amount of nucleic acid bonded to the nucleic acid probe 3 is decreased. As a result, the redox potential of the labeling substance 4 is easily detected.

Bringing of the labeling substance into the reaction field may be carried out by immobilizing releasably the labeling substance on at least one surface of the substrate in contact with the reaction field of the nucleic acid probe immobilized substrate, and may be dissolved in the reaction solution in advance. Further, when the reaction field is formed inside a flow channel, it may be releasably immobilized to at least one part of an inner wall of a flow channel. When a plurality of nucleic acid probes are immobilized in the flow channel, it is preferred to be immobilized so as to uniformly correspond to the respective nucleic acid probes. For example, it is preferred to be immobilized by an equivalent amount to inside the flow channel at positions corresponding to the respective nucleic acid probes, that is, within the respective reaction fields where the respective amplification reactions are to be performed or in surroundings thereof, etc.

That is, detection or quantification of the target nucleic acid may be performed by monitoring the signal from the labeling substance, or detecting the signal at two or more time points.

The signal regarding the target nucleic acid in the third embodiment may be measured, for example, by taking the signal from the labeling substance as a current value in a potential function. For example, cyclic voltammetry may be used for the measurement potential to be given is chosen depending on the labeling substance used and this potential may be swept as a triangle wave. Here, an electric signal that reflects presence of the labeling substance from the potential to be given and a current may be obtained.

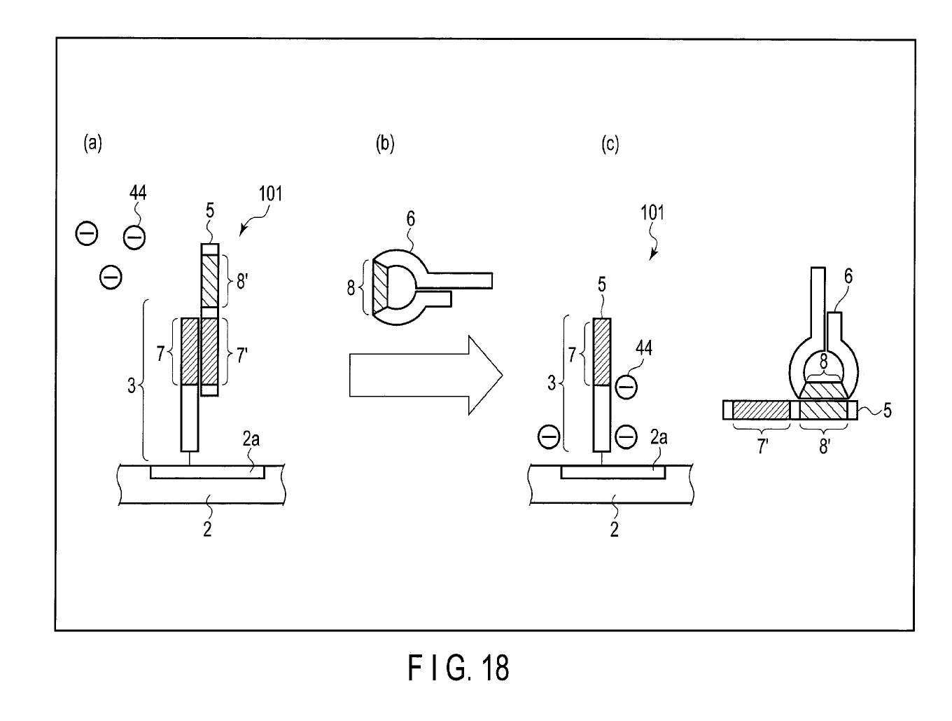

A change over time of an electric signal obtained when the nucleic acid probe to which the covering nucleic acid chain is bonded, the target nucleic acid to be detected, and a primer set for amplifying the target nucleic acid are present in the reaction field and the reaction field is maintained under the isothermal amplification reaction condition is shown in FIG. 18 as an image diagram. As shown in FIG. 18, when the signals from the electrode are continuously monitored, the potential detected at the time of forming the reaction field by the reaction solution is relatively low, and is shown as region a in a waveform of FIG. 18. Here, as shown in FIG. 17(a), the covering nucleic acid chain is bonded to the nucleic acid probe. Accordingly, the labeling substance is repelled by the negative charge increased by the covering nucleic acid chain to be kept away from the electrode. After a predetermined time has elapsed, the electric signal is rapidly increased (FIG. 18, region b). It indicates that the amplification of the target nucleic acid proceeds in the reaction field under the isothermal amplification reaction condition and the covering nucleic acid chain is rapidly desorbed from the nucleic acid probe at a certain point. Then, the electric signal becomes gradually large, but is stabilized at a certain level (FIG. 18, region c).

The detection and quantification of the target nucleic acid may be performed based on waveforms obtained by detection over time or detection at multiple time points of the electric signal derived from such a labeling substance. For example, the electric signal from the labeling substance may be monitored over desired time from start of the isothermal amplification reaction, or may be measured at two or more time points desired from the start of the isothermal amplification reaction. The detection and quantification of the target nucleic acid may be performed based on obtained results, for example, based on a change in waveform, or by comparison with a predetermined threshold value or by comparison with data from a control probe obtained in advance or at the same time, for example, by comparison with a waveform or a numerical value.

For example, the target nucleic acid may be detected or quantified in accordance with time until an obtained peak potential is lower than the predetermined threshold value, or a difference with the peak potential at a certain time point. Otherwise, a calibration curve may be prepared in advance.

For example, a method in which the target nucleic acid is quantified as compared to a control may include the following processes. As the control, for example, a single strand nucleic acid probe that is not hybridized with a covering nucleic acid chain (hereinafter, referred to as a ┌control probe┘) is prepared. This is immobilized to at least one surface of the substrate in contact with the reaction field independent of a reaction field for an immobilizing sample. This control probe may be present in a vessel such as the flow channel in which the reaction field in which a test is performed on the sample is present or may be present in another vessel. Measurement of the signal from the control probe may be performed in the same order as a procedure for measuring the signal from the nucleic acid probe for detecting the target nucleic acid (i.e., a probe for detection) except that the sample is not present in the reaction field for the control. By performing the isothermal amplification reaction for the respective control probe and probe for detection, and taking an electric signal from the labeling substance, the signal may be measured as the current value in the potential function.

For example, cyclic voltammetry may be used for the measurement of the current value. Here, a potential to be given may be chosen depending on the used labeling substance and may be swept as a triangle wave. A graph of an oxidation direction of the potential and a current to be given may show a waveform as shown in FIG. 18. A peak current and a peak potential are obtained from this graph. Subsequently, the difference in peak potentials obtained from the control probe and the probe for detection, respectively, is obtained as Δ peak potential. For example, electric signals such as peak current, peak potential and Δ peak potential are managed and measured by a computer and are capable of being calculated arbitrarily. Measurement or calculation of the Δ peak potential may be performed by any known method from the obtained electric signals. A concentration of nucleic acid may be identified depending on the time until the obtained Δ peak potential is lower than the predetermined threshold value, or a difference with the Δ peak potential at a certain time point.

The method for detecting the target nucleic acid according to the third embodiment may include the following processes:

performing an isothermal amplification using a primer set for forming an amplification product including a first sequence using the target nucleic acid as a template, under presence of a nucleic acid probe that is immobilized on a solid phase by a nucleic acid chain including a second sequence different from the first sequence, and that comprises a covering nucleic acid chain bonded via hybridization to the second sequence in a second sequence bonding region, the covering nucleic acid chain including a first sequence bonding region complementary to the first sequence and a second sequence bonding region complementary to the second sequence;

competing the amplification product formed in the isothermal amplification reaction with the nucleic acid probe to thereby desorb the covering nucleic acid chain from the nucleic acid probe and to bind the amplification product to the covering nucleic acid chain through hybridization of the first sequence in the first sequence bonding region; and

monitoring a signal or detecting the signal at two or more time points from a labeling substance in which possibility for detecting the signal is inhibited due to the bonding of the nucleic acid probe and the covering nucleic acid chain, under the isothermal amplification reaction condition.

The concentration of nucleic acid which is detectable by the method for detecting the nucleic acid of the third embodiment may be 1 aM to 1 nM.

According to the method of the embodiment, the target nucleic acid can be quantified simply. In addition, more target nucleic acids is capable of being tested in a time shorter than that in prior art. Further, an error may hardly occur in selection of the sample. In addition, according to the third embodiment, a concentration of the target nucleic acid may be measured simply at higher accuracy than that of the second embodiment. Constitution of the method for detecting the target nucleic acid of the third embodiment and the probe immobilized substrate used herein may be the same as those of the above-described second embodiment except that the labeling substance is present in the reaction solution and is not bonded to the nucleic acid probe. Accordingly, it is possible to incorporate any constitution, combination, etc., described for the above-described second embodiment into the third embodiment, or to modify a part of the above-described third embodiment.

### 4. Fourth Embodiment

An example of the method for detecting the target nucleic acid of the above-described first embodiment, in which each of the sequence of the nucleic acid probe and the covering nucleic acid chain are the sequences of the above (b) is described below as the fourth embodiment. In this embodiment, similar to the third embodiment, an electrochemically active substance having a negative charge in a reaction field is used as a labeling substance. The fourth embodiment may be performed in the same manner using the same nucleic acid probe immobilized substrate as the third embodiment except that each of the sequence of the nucleic acid probe and the covering nucleic acid chain are the sequences of the above (b).

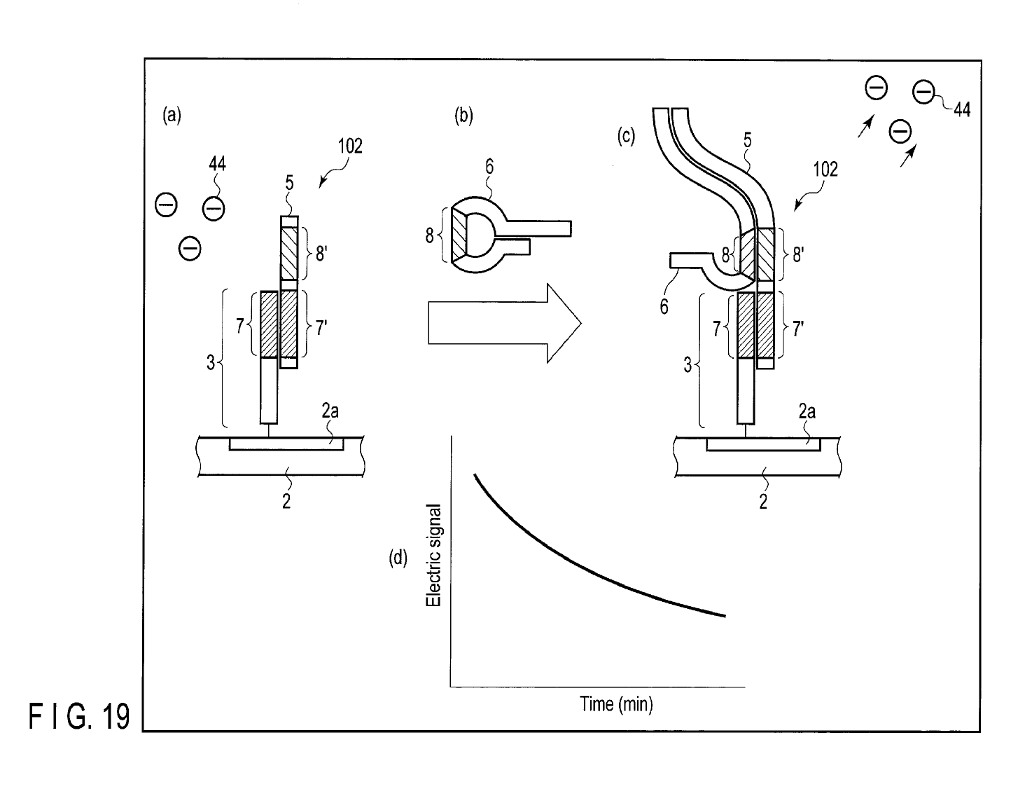

An example of the probe immobilized substrate used for the fourth embodiment is described with reference to FIG. 19. A basic structure of the probe immobilized substrate is the same as that shown in FIG. 17A. The nucleic acid probe 3 is immobilized on the electrode 2a of the substrate 2 of the probe immobilized substrate 102. The covering nucleic acid chain 5 is bonded to the nucleic acid probe 3 (FIG. 19(a)). The labeling substance 44 is present in the reaction field in which they are present. When the reaction field is maintained under an isothermal amplification condition, an amplification reaction using a target nucleic acid as a template proceeds, such that an amplification product 6 is formed over time (FIG. 19(b)). The formed amplification product 6 is bonded to the covering nucleic acid chain 5, and while maintaining this state, elongation of the covering nucleic acid chain 5 using the amplification product 6 as a template is started. Accordingly, the negative charge derived from the nucleic acid bonded to the nucleic acid probe 3 becomes larger than that of the initial covering nucleic acid chain 5 (FIG. 19(c)). As a result, an electric signal obtained by an electrode 2a becomes smaller over time. This is because repulsion between the negative charges of the labeling substance 44 and the negative charge of the nucleic acid bonded to the nucleic acid probe 3 becomes large.

A change over time of the electric signal obtained from the electrode which is continuously monitored by using the probe immobilized substrate 102 is shown in FIG. 19(d) as an image diagram. As shown herein, the obtained electric signal is decreased depending on formation and an increase of the amplification product from beginning of the amplification reaction. That is, detection of a detectable signal produced by the labeling substance is inhibited by a presence or an increase in abundance of the nucleic acid which is bonded to the nucleic acid probe.

By using this phenomenon, it is possible to detect the presence of the target nucleic acid or to measure an abundance of the target nucleic acid based on the electrical signal detected from the labeling substance.

The method for detecting the target nucleic acid according to the fourth embodiment may include the following processes;

performing an isothermal amplification using a primer set for forming an amplification product including a first sequence using the target nucleic acid as a template, under presence of a nucleic acid probe that is immobilized on a solid phase by a nucleic acid chain including a second sequence different from the first sequence, and that comprises a covering nucleic acid chain bonded via hybridization to the second sequence in a second sequence bonding region, the covering nucleic acid chain including a first sequence bonding region complementary to the first sequence and a second sequence bonding region complementary to the second sequence;

bonding the first sequence bonding region of the covering nucleic acid chain with the first sequence of the amplification product formed in the isothermal amplification reaction via hybridization and elongating the covering nucleic acid chain using the amplification product as a template; and

monitoring a signal or detecting the signal at two or more time points from a labeling substance in which possibility for detecting the signal is inhibited due to the elongating of the covering nucleic acid chain using the amplification product as a template, under the isothermal amplification reaction condition.

The concentration of nucleic acid which is detectable by the method for detecting the nucleic acid of the fourth embodiment may be 1 aM to 1 nM.

According to the method of the embodiment, the target nucleic acid may be easily quantified. In addition, more target nucleic acids is capable of being tested in a time shorter than that in prior art. Further, an error may hardly occur in selection of the sample.

The labeling substance used in the fourth embodiment may be an electrochemically active substance having a negative charge in the reaction field among the substances in which signal detection is inhibited by elongation of the covering nucleic acid chain using the amplification product as the template, similar to the labeling substance used in the third embodiment. Examples of the specific substance are as described above. For example, Tm values and sequence length of the nucleic acid probe and the covering nucleic acid chain are set so as to maintain hybridization even at a salt concentration of the reaction field and a temperature at the time of the amplification reaction, and to maintain hybridization even in an environment in which the amplification product from the target nucleic acid is present.

In regard to criteria for setting the nucleic acid probe 3 and the covering nucleic acid chain 5 and for determining the isothermal amplification reaction condition, the nucleic acid probe, the covering nucleic acid chain, and a base sequence length of the amplification reaction product, and the isothermal amplification reaction condition, for example, any one of a temperature and a salt concentration may be set first, and other conditions may be set so as to satisfy the above two conditions.

The constitution of the method for detecting the target nucleic acid of the fourth embodiment and the probe immobilized substrate used herein are the same as the above-described third embodiment except that the bonding with the covering nucleic acid chain is maintained regardless of the presence of the amplification product, and in addition thereto, may be the same as the above-described second embodiment except that the labeling substance is present in the reaction solution and is not bonded to the nucleic acid probe. It is possible to incorporate any constitution, combination, etc., described for the above-described second and third embodiments into the fourth embodiment, or to modify a part of the above-described third embodiment. In other words, with respect to any of the embodiments described herein, any of the constitution described in the other embodiments and the combination thereof, and parts thereof, etc., may be incorporated into each other, or any part thereof may be modified to replace a part of another embodiment.

### 5. Assay Kit for Detecting Target Nucleic Acid

The present embodiment may be provided as an assay kit for performing the above-described target nucleic acid detection. The assay kit for detecting a target nucleic acid may comprise a nucleic acid probe immobilized substrate and a primer set. Otherwise, the kit for detecting the target nucleic acid may comprise a primer set, a nucleic acid probe immobilized substrate, and a labeling substance that produces a detectable signal.

Details for these nucleic acid probe immobilized substrate, primer set, and labeling substance are the same as described above. All of the components included in the kit for detecting the target nucleic acid may be included in the assay kit in an independent form, and in order to be introduced into a corresponding reaction field formed by a presence of a reaction solution at the time of using the kit, all or a part of the components other than the nucleic acid probe immobilized substrate may be included in the assay kit in a state where they are immobilized on at least one surface of the nucleic acid probe immobilized substrate.

The primer set may be releasably immobilized to the nucleic acid probe immobilized substrate or may be included in the kit without being immobilized to the nucleic acid probe immobilized substrate.

In addition, the assay kit may include a further reaction reagent for amplification of the target nucleic acid besides the nucleic acid probe immobilized substrate and the primer set. The further reaction reagent may be, for example, an enzyme, dNTP and/or a buffer.

The assay kit of the embodiment can detect the target nucleic acid simply, at higher accuracy, and can further detect the target nucleic acid quantitatively. The target nucleic acid can be tested in a time shorter than that in prior art. An error may hardly occur in selection of the sample.

The method for detecting the nucleic acid using the above-described assay kit, the probe immobilized substrate may be performed using, for example, a device for detecting a target nucleic acid as described below.

### 6. Device for Detecting Target Nucleic Acid

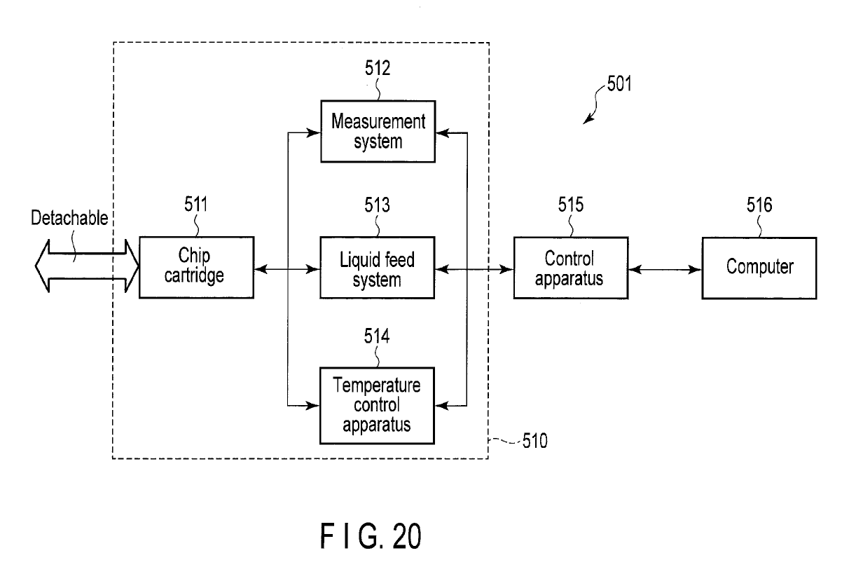

According to an embodiment, the device for detecting the target nucleic acid may be provided. FIG. 20 is a block illustration showing an example of the embodiment of the device for detecting the target nucleic acid. A device for detecting a target nucleic acid 501 for detecting a target nucleic acid is provided with a measurement unit 510, a control apparatus 515 that controls the measurement unit 510, and a computer 516 that controls the control apparatus 515. The measurement unit 510 comprises a chip cartridge 511 detachably arranged therefrom and performing a reaction thereon, a measurement system 512 that obtains a signal from the chip cartridge 511, a liquid feed system 513 that sends and/or takes out liquid to/from the chip cartridge 511, and a temperature control apparatus 514 that controls a temperature of the chip cartridge 511.

The device for detecting the target nucleic acid 501 may have the following constitution according to constitutions of the labeling substance and the chip cartridge to be used, etc.

(1) When a reaction solution is provided from outside of the chip cartridge and a labeling substance that emits an electric signal is used,

the device for detecting the target nucleic acid 501 is provided with the chip cartridge 511, the measurement system 512 electrically connected to the chip cartridge 511, the liquid feed system 513 that is physically connected to a flow channel installed in the chip cartridge 511 through an interface part and that sends a reagent to the chip cartridge 511, the reagent being stored in a container arranged outside the chip cartridge 511, and the temperature control apparatus 514 that controls a temperature of the chip cartridge 511.

(2) When the reaction solution is provided inside the chip cartridge and the labeling substance that emits an electric signal is used,

the device for detecting the target nucleic acid 501 is provided with the chip cartridge 511, the measurement system 512 electrically connected to the chip cartridge 511, the liquid feed system 513 in which the reagent stored in the chip cartridge 511 is moved to a predetermined position by physically opening and closing a valve installed in the chip cartridge 511 through the interface part, and the temperature control apparatus 514 that controls a temperature of the chip cartridge 511.

(3) When a reaction solution is provided from outside of the chip cartridge and a labeling substance that emits an optical signal is used,

the device for detecting the target nucleic acid 501 is provided with the chip cartridge 511, the measurement system 512 for measuring an optical signal from the chip cartridge 511, the liquid feed system 513 that is physically connected to a flow channel installed in the chip cartridge 511 through the interface part and that sends the reagent to the chip cartridge 511, the reagent being stored in the container arranged outside the chip cartridge 511, and the temperature control apparatus 514 that controls a temperature of the chip cartridge 511.

(4) When the reaction solution is provided inside the chip cartridge and the labeling substance that emits an optical signal is used,

the device for detecting the target nucleic acid 501 is provided with the chip cartridge 511, the measurement system 512 for measuring the optical signal from the chip cartridge 511, the liquid feed system 513 in which the reagent stored in the chip cartridge 511 is moved to a predetermined position by physically opening and closing the valve installed in the chip cartridge 511 through the interface part, and the temperature control apparatus 514 that controls a temperature of the chip cartridge 511.

For example, an example of the device for detecting the target nucleic acid shown in the above (1) is described below.

The chip cartridge 511 includes, for example, a multi-nucleic-acid amplification detection reaction tool 91 and a cover 301 immobilized on the reaction tool 91 shown in FIG. 14. A space formed by the reaction tool 91 and the cover 301 forms a flow channel in which a left hand side is upstream and a right hand side is downstream while facing each other. An inner part of the flow channel corresponds to a reaction part, and the reaction field is formed therein to perform desired amplification and detection reactions. A feeding port for feeding liquid is provided on an upper surface of the cover 301 on the upstream side (not shown). A discharging port for discharging the liquid is installed on an upper surface of the cover 301 on the downstream side (not shown).

The measurement system 512 applies a voltage to the electrode of the chip cartridge 511 and simultaneously receives an electrical signal from the chip cartridge 511 and sends it to the control apparatus 515.

The liquid feed system 513 may include a container for receiving liquid such as a reaction solution, etc., and an interface with the chip cartridge 511. The liquid feed system 513 sends the liquid in the container into the chip cartridge 511 via the interface as needed by the control of the control apparatus 515.

The temperature control apparatus 514 controls a temperature of at least the reaction part in the chip cartridge 511 so as to satisfy a temperature condition for the amplification and detection reactions. For this purpose, the temperature control apparatus 514 may include, for example, a heater and/or a Peltier element, etc. The temperature control apparatus 514 is controlled by the control apparatus 515 and controls the temperature of the reaction part in the chip cartridge 511.

The control apparatus 515 is electrically connected to the measurement system 512, the liquid feed system 513, the temperature control apparatus 514, and the computer 516. The control apparatus 515 controls the measurement system 512, the liquid feed system 513 and the temperature control apparatus 514 according to a program provided in the computer 516, detects a signal obtained from the measurement system 512, and stores the signal as measurement data.

The computer 516 controls the control apparatus 515 by providing a control condition parameter to the control apparatus 515, and simultaneously executes an analysis process based on the measurement data stored in the control apparatus 515 to detect and/or quantify the nucleic acid.

The nucleic acid detection by the device for detecting a nucleic acid may be performed, for example, as follows. First, an operator injects a sample into the reaction part of the chip cartridge 511, inserts the chip cartridge 511 into the measurement unit 510, and starts detection by the device for detecting the target nucleic acid 501. A container of the liquid feed system 513 is filled with a reaction solution including a labeling substance in advance. The computer operates the liquid feed system 213 according to the program stored in advance in the computer, and the reaction solution is sent to the reaction part of the chip cartridge 511. Under the control of the computer and the control apparatus, the temperature control apparatus 514 adjusts the temperature of the reaction field to start the isothermal amplification reaction. Under the control of the computer and control apparatus, the measurement system 512 acquires an electric signal from the reaction part. The electric signal obtained by the measurement system 512 is sent to the control apparatus, and stored as data. The stored data are called and processed and interpreted by the computer according to the program to obtain information about the nucleic acid to be detected, i.e., a detection result and/or a quantification result, included in the sample. The result obtained by the computer may be outputted to a display, a printer, or the like, provided by the computer as desired, or may be stored in the computer.

Although an example of a device for detecting an electrical signal is shown as an example of the above-described embodiment, it is also possible to use a device for detecting a target nucleic acid in the same manner even at the time of using a labeling substance which produces an optical signal. In this case, the device for detecting a target nucleic acid may have the same constitution as described above except that the measurement system 512 is configured to detect the optical signal. For example, when a fluorescent substance is used as a labeling substance, the measurement system 512 may be provided with a light irradiation unit which irradiates an excitation light to the reaction part, a sensing unit which obtains fluorescence from the labeling substance as an optical signal, and a photoelectric conversion unit which converts the optical signal into an electric signal, etc.

The device for detecting the target nucleic acid according to the embodiment is capable of detecting or quantifying the target nucleic acid simply at higher accuracy than that of the prior art. Further, according to the device for detecting the target nucleic acid, the target nucleic acid is capable of being tested in a time shorter than that in prior art.

### EXAMPLES

### Example 1

An example of producing and using an array-type nucleic acid probe immobilized substrate 1 for electrochemical detection having the same configuration as the reaction tool shown in FIG. 14 will be explained. Any array-type nucleic acid probe immobilized substrate for electrochemical detection includes a primer set immobilized to a primer-immobilized region and a probe DNA used as a nucleic acid probe immobilized to a probe-immobilized region in close vicinity to the primer-immobilized region. The probe-immobilized region was arranged on an electrode and used as a sensor for detecting a current response generated depending on the existence of hybridization.

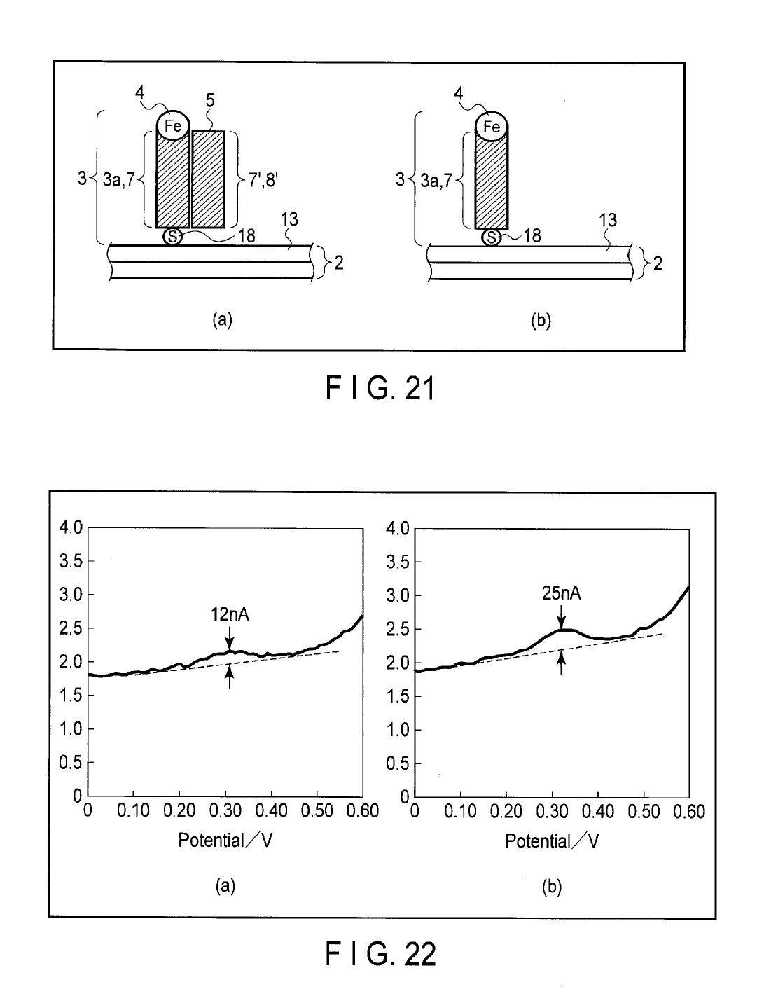

FIG. 21(a) and FIG. 21(b) are partially enlarged illustrations schematically showing the probe-immobilized region of the array-type nucleic acid probe immobilized substrate. The probe DNA used as the nucleic acid probe 3 is immobilized to the probe-immobilized region 13 arranged on the electrode. The nucleic acid probe 3 includes a nucleic acid chain 3a, a labeling substance 4 imparted to one of terminals of the nucleic acid chain 3a, and a terminal-modifier 18 bonded to the other terminal. A covering nucleic acid chain 5 is bonded to the nucleic acid probe 3. The sequence of the nucleic acid chain 3a and the sequence of the covering nucleic acid chain 5 are complementary to each other. The detection signal from the labeling substance, which can be detected by desorption from the nucleic acid probe 3 of the covering nucleic acid chain 5, is detected by a sensor including the electrode on which the nucleic acid probe 3 is immobilized.

FIG. 21(b) is a partially enlarged illustration schematically showing the probe-immobilized region of the array-type nucleic acid probe immobilized substrate in a case of immobilizing the nucleic acid probe 3 to which the covering nucleic acid chain is not bonded. FIG. 21(b) is similar to FIG. 21(a) except a feature that the covering nucleic acid chain 5 is not bonded to the nucleic acid probe 3.

(1) Preparation of Chip Material

A thin film of titanium and gold was formed on the surface of Pyrex (trademark) glass by sputtering. After that, an electrode of titanium and gold was formed on the glass surface by etching. Furthermore, an insulating film was applied thereon, and a circular window and a rectangular window were opened in the insulating film by etching to expose a working electrode, a counter-electrode, a reference electrode and a prober contact portion. This was set as a chip material for an array-type primer probe chip.

(2) Preparation of Array-Type Nucleic Acid Probe Immobilized Substrate

First, a sequence (A) was prepared as a nucleic acid chain included in the nucleic acid probe. 3′ terminal of the sequence (A) was labeled with thiol and 5′ terminal of the sequence (A) was labeled with ferrocene. A nucleic acid chain composed of a sequence (B) was prepared as a covering nucleic acid chain and hybridized with the nucleic acid probe including the sequence (A) to prepare a double-stranded nucleic acid probe (A). Similarly, a sequence (A) was prepared as a nucleic acid chain included in the nucleic acid probe. 3′ terminal of the sequence (A) was labeled with thiol and 5′ terminal of the sequence (A) was labeled with ferrocene. A single-stranded nucleic acid probe (B) was formed without bonding the covering nucleic acid chain. The double-stranded nucleic acid probe can also be formed by adding the covering nucleic acid chain on the base plate on which the single-stranded nucleic acid probe is immobilized.

The double-stranded nucleic acid probe (A) and the single-stranded nucleic acid probe (B) were immobilized on two working electrodes, respectively, for each type. Base sequences of the nucleic acid chain used are shown in Table 1.

TABLE 1

| Sequence | SEQ ID NO. | Sequence |
|---|---|---|
| (A) | 1 | GTTGGTGTGCCACTAGTTCC |
| (B) | 2 | GGAACTAGTGGCACACCAAC |

The immobilization was executed in a manner explained below. Probe solutions (A) and (B) containing 3 μM of the double-stranded nucleic acid probe (A) and 3 μM of the single-stranded nucleic acid probe (B), respectively, were prepared. 100 nL of the probe solutions were spotted on the respective working electrodes. Drying was performed at 40° C., followed by performing washing with ultrapure water. After that, the ultrapure water left on the surfaces of the working electrodes was removed and the nucleic acid probes were immobilized on two working electrodes of the chip material. FIG. 21(a) schematically illustrates the double-stranded nucleic acid probe (A) immobilized on the probe-immobilized region 13 arranged on the electrode. FIG. 21(b) schematically illustrates the single-stranded nucleic acid probe (B) immobilized on the probe-immobilized region 13 arranged on the electrode.

(3) Detection of Electrochemical Signal

Electrochemical responses from the nucleic acid probe (A) and the nucleic acid probe (B) were measured with electrochemical analyzer ALS660a. Cyclic voltammetry was applied as a measurement method and a potential sweep rate was 100 V/s.

The measurement results are shown in FIG. 22(a) and FIG. 22(b). In these graphs, a horizontal axis represents a potential (V) and a vertical axis represents a current (nA). FIG. 22(a) shows a result detected from the electrode on which the nucleic acid probe (A) was immobilized, and FIG. 22(b) shows a result detected from the electrode on which the nucleic acid probe (B) was immobilized. Since the nucleic acid probe was double-stranded by hybridization between the nucleic acid probe and the covering nucleic acid chain, the current value obtained by the nucleic acid probe to which the covering nucleic acid chain was bonded was approximately half the current value obtained by the nucleic acid probe to which the covering nucleic acid chain was not bonded. It was clarified from this result that the signal from the labeling substance in the nucleic acid probe can be masked by hybridization of the covering nucleic acid chain to the nucleic acid probe.

(4) Preparation of Primer Set

Next, a primer DNA to be used as a primer set 12 was prepared. The primer DNA to be used is a primer set 12 for amplification in the loop-mediated isothermal amplification (LAMP) method. Base sequences of primer DNAs used are shown in Table 2.

TABLE 2

| SEQ | | |
|---|---|---|
| ID | | |
| NO. | | Sequence |
| 3 | F3 | GAGATATTATTTTCAATGGGATAGAAC |
| 4 | B3 | CAATGCTCTATTTGTTTGCCATG |
| 5 | FIP | GAACATCATCTGGATCTGTACCAACCATCTCATACTGGAACTA |
| | | GTGGC |
| 6 | BIP | CTGTGCCAGTACACTTACTAAGAGTGTTAGTCTACATGGTTTA |
| | | CAATC |
| 7 | LPF | TGGTATATATTTGTTGGTGTGCC |
| 8 | | ACAGGTGATGAATTTGCTACAGG |

As for concentrations of primers used for the LAMP amplification reaction, the concentration of FIP primer and BIP primer was 1.6 μM, the concentration of F3 primer and B3 primer was 0.12 μM, and the concentration of LPF primer was 0.4 μM.

(5) Preparation of LAMP Reaction Solution

A composition of a LAMP reaction solution is presented below in Table 3.

TABLE 3

| Components | Concentration | |
|---|---|---|
| Tris-HCl (pH 8.0) | 20 | mM |
| KCl | 10 | mM |
| MgSO4 | 8 | mM |
| (NH4)2SO4 | 10 | mM |
| Tween20 | 0.1% | |
| dNTPs | 1.4 | mM each |
| Bst polymerase | 8 | unit |

0.8 M betaine was added in a general LAMP reaction, but was not used since betaine inhibits a reaction in an electrochemical measurement. 10⁵ copy/μL plasmid (length: approximately 4 kbp) was used as a template and the LAMP amplification reaction was carried out at 63° C. The plasmid used was obtained by inserting a sequence represented by SEQ ID No. 9 shown in Table 3-2 (VP gene of parvo virus, with a length of 1000 bp) into a pMA vector. SEQ ID No. 9 partially includes polynucleotide of SEQ ID No. 2. A portion corresponding to the sequence of SEQ ID No. 2 of Table 4 is underlined.

TABLE 4
Parvo VP2 gene

AAACGCTAATACGACTCACTATAGGGCGATCTACGGGTACTTTCA
ATAATGAGACGGAATTTAAATITTTGGAAAACGGATGGGTGGAAA
TCACAGCAAACTCAAGCAGACTTGTACATTTAAATATGCCAGAAA
GTGAAAATTATAGAAGAGTGGTTGTAAATAATTTGGATAAAACTG
CAGTTAACGGAAACATGGCTTTAGATGATACTCATGCACAAATTG
TAACACCTTGGTCATTGGTTGATGCAAATGCTTGGGGAGTTTGGT
TTAATCCAGGAGATTGGCAACTAATTGTTAATACTATGAGTGAGT
TGCATTTAGTTAGTTTTGAACAAGAAATTTTTAATGTTGTTTTAA
AGACTGTTTCAGAATCTGCTACTCAGCCACCAACTAAAGTTTATA
ATAATGATTTAACTGCATCATTGATGGTTGCATTAGATAGTAATA
ATACTATGCCATTTACTCCAGCAGCTATGAGATCTGAGACATTGG
GTTTTTATCCATGGAAACCAACCATACCAACTCCATGGAGATATT
ATTTTCAATGGGATAGAACATTAATACCATCTCATACTGGAACTA
GTGGCACACCAACAAATATATACCATGGTACAGATCCAGATGATG
TTCAATTTTATACTATTGAAAATTCTGTGCCAGTACACTTACTAA
GAACAGGTGATGAATTTGCTACAGGAACATTTTTTTTTGATTGTA
AACCATGTAGACTAACACATACATGGCAAACAAATAGAGCATTGG
GCTTACCACCATTTCTAAATTCTTTGCCTCAAGCTGAAGGAGGTA
CTAACTTTGGTTATATAGGAGTTCAACAAGATAAAAGACGTGGTG
TAACTCAAATGGGAAATACAAACTATATTACTGAAGCTACTATTA
TGAGACCAGCTGAGGTTGGTTATAGTGCACCATATTATTCTTTTG
AGGCGTCTACACAAGGGCCATTTAAAACACCCTTCCCTTTAGTGA
GGGTTAATAA (SEQ ID No. 9)

(5) LAMP Amplification Using Array-Type Nucleic Acid Probe Immobilized Substrate for Electrochemical Detection and Detection of Target Nucleic Acid Using Nucleic Acid Probe

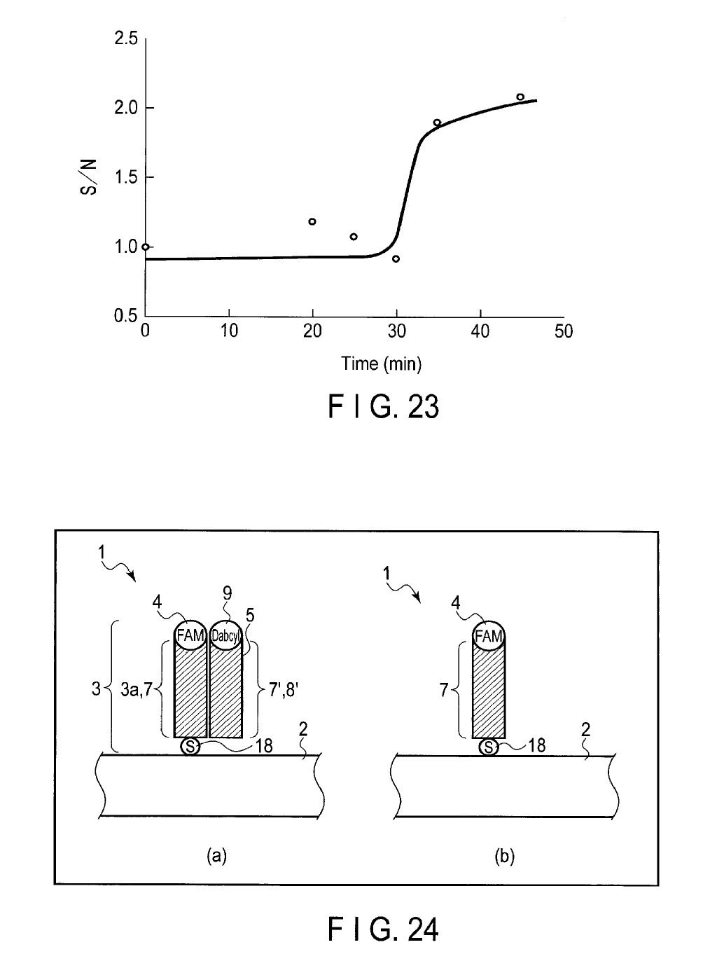

An oxidation current of ferrocene was measured while executing the LAMP amplification on a chip on which the nucleic acid probe was immobilized. The obtained results are shown in FIG. 23. As a result of plotting a current ratio (S/N) of a chip to which a template was added and a chip to which a template was not added, S/N started varying at the thirtieth minute and varied to 2 at the forty-fifth minute. It was clarified from this result that the target nucleic acid can be detected quantitatively by an electrochemical method of monitoring the current of ferrocene and monitoring the time of S/N variation.

### Example 2

An example of producing and using an array-type nucleic acid probe immobilized substrate 1 for detection of fluorescence will be hereinafter explained.

The array-type nucleic acid probe immobilized substrate 1 for detection of fluorescence was prepared similarly to Example 1 except a feature that any array-type nucleic acid probe immobilized substrate uses a fluorescent substance as a labeling substance and further uses a modifying substance for assisting inhibition of detectability of the signal from the fluorescent substance by the covering nucleic acid chain. The array-type nucleic acid probe immobilized substrate for optical detection includes a primer set immobilized to a primer-immobilized region and a probe DNA used as a nucleic acid probe immobilized to a probe-immobilized region in close vicinity to the primer-immobilized region. The probe-immobilized region was arranged on an electrode, but the detection of the detection signal was executed by optically measuring a fluorescent intensity from the labeling substance.

FIG. 24(a) and FIG. 24(b) are partially enlarged illustrations schematically showing the probe-immobilized region of the array-type nucleic acid probe immobilized substrate. The nucleic acid probe 3 shown in FIG. 24(a) includes a nucleic acid chain 3a, a labeling substance 4 imparted to one of terminals of the nucleic acid chain 3a, and a terminal-modifier bonded to the other terminal. The covering nucleic acid chain 5 and the nucleic acid chain 3a have mutually complementary sequences. When the covering nucleic acid chain 5 and the nucleic acid chain 3a are hybridized, a modifying substance is imparted to one of terminals of the covering nucleic acid chain 5, which is opposed to the terminal of the nucleic acid chain 3a to which the labeling substance 4 is bonded. The covering nucleic acid chain 5 is not bonded to the nucleic acid probe 3 shown in FIG. 24(b).

(1) Preparation of Chip Material

A thin film of titanium and gold was formed on the surface of Pyrex (trademark) glass by sputtering. This was set as a chip material for an array-type primer probe chip.

(2) Preparation of Array-Type Nucleic Acid Probe Immobilized Substrate

First, a sequence (A) was prepared as a nucleic acid chain included in the nucleic acid probe. 3′ terminal of the sequence (A) was labeled with thiol and 5′ terminal of the sequence (A) was labeled with FAM. This was set as a nucleic acid probe (C). A nucleic acid chain composed of a sequence (B) was prepared as a covering nucleic acid chain and hybridized with a nucleic acid probe (C) including the sequence (A) to prepare a double-stranded nucleic acid probe (C). Similarly, a sequence (A) was prepared as a nucleic acid chain included in the nucleic acid probe. 3′ terminal of the sequence (A) was labeled with thiol and 5′ terminal of the sequence (A) was labeled with FAM. A single-stranded nucleic acid probe (D) was formed without bonding the covering nucleic acid chain.

The double-stranded nucleic acid probe (C) and the single-stranded nucleic acid probe (D) were immobilized on two working electrodes, respectively, for each type. A base sequence of the nucleic acid used is shown in Table 5.

TABLE 5

| Sequence | SEQ ID NO. | Sequence |
|---|---|---|
| (A) | 1 | GTTGGTGTGCCACTAGTTCC |
| (B) | 2 | GGAACTAGTGGCACACCAAC |

The immobilization was executed in a manner explained below. Probe solutions (C) and (D) containing 3 μM of the double-stranded nucleic acid probe (C) and 3 μM of the single-stranded nucleic acid probe (D), respectively, were prepared. 100 nL of the probe solutions were spotted on the respective working electrodes. Drying was performed at 40° C., followed by performing washing with ultrapure water. After that, the ultrapure water left on the surfaces of the working electrodes was removed and the nucleic acid probes were immobilized on two working electrodes of the chip material. FIG. 24(a) schematically illustrates the double-stranded nucleic acid probe (C) immobilized on the probe-immobilized region 13 arranged on the electrode. FIG. 24(b) schematically illustrates the single-stranded nucleic acid probe (D) immobilized on the probe-immobilized region 13 arranged on the electrode.

(3) Detection of Optical Signal

Fluorescence intensities from the nucleic acid probes were measured. As a result, the fluorescence intensity from the double-stranded nucleic acid probe (C) was measured to be approximately double the measured fluorescence intensity from the single-stranded nucleic acid probe (D).

(4) Preparation of Primer Set

Next, a primer DNA to be used as a primer set 12 was prepared. The primer DNA to be used is a primer set for amplification in the LAMP method. Base sequences of the primer DNA used are shown in Table 6.

TABLE 6

| SEQ | | |
|---|---|---|
| ID | | |
| NO. | | Sequence |
| 3 | F3 | GAGATATTATTTTCAATGGGATAGAAC |
| 4 | B3 | CAATGCTCTATTTGTTTGCCATG |
| 5 | FTP | GAACATCATCTGGATCTGTACCAACCATCTCATACTGGAACTA |
| | | GTGGC |
| 6 | BIP | CTGTGCCAGTACACTTACTAAGAGTGTTAGTCTACATGGTTTA |
| | | CAATC |
| 7 | LPF | TGGTATATATTTGTTGGTGTGCC |
| 8 | | ACAGGTGATGAATTTGCTACAGG |

As for concentrations of primers used for the LAMP amplification reaction, the concentration of FIP primer and BIP primer was 1.6 μM, the concentration of F3 primer and B3 primer was 0.12 μM, and the concentration of LPF primer was 0.4 μM.

(5) Preparation of LAMP Reaction Solution

A composition of a LAMP reaction solution is presented below in Table 7.

TABLE 7

| Components | Concentration | |
|---|---|---|
| Tris-HCl (pH 8.0) | 20 | mM |
| KCl | 10 | mM |
| MgSO4 | 8 | mM |
| (NH4)2SO4 | 10 | mM |
| Tween20 | 0.1% | |
| dNTPs | 1.4 | mM each |
| Betain | 0.8M | |
| Bst polymerase | 8 | unit |

In the fluorescence measurement, 0.8M betaine was added, similarly to a general LAMP reaction. 10⁵ copy/μL plasmid (length: approximately 4 kbp) was used as a template and the LAMP amplification reaction was carried out at 63° C. The same plasmid as that in Example 1 was used.

(6) LAMP Amplification Using Array-Type Nucleic Acid Probe Immobilized Substrate for Fluorescence Detection and Detection of Target Nucleic Acid Using Nucleic Acid Probe

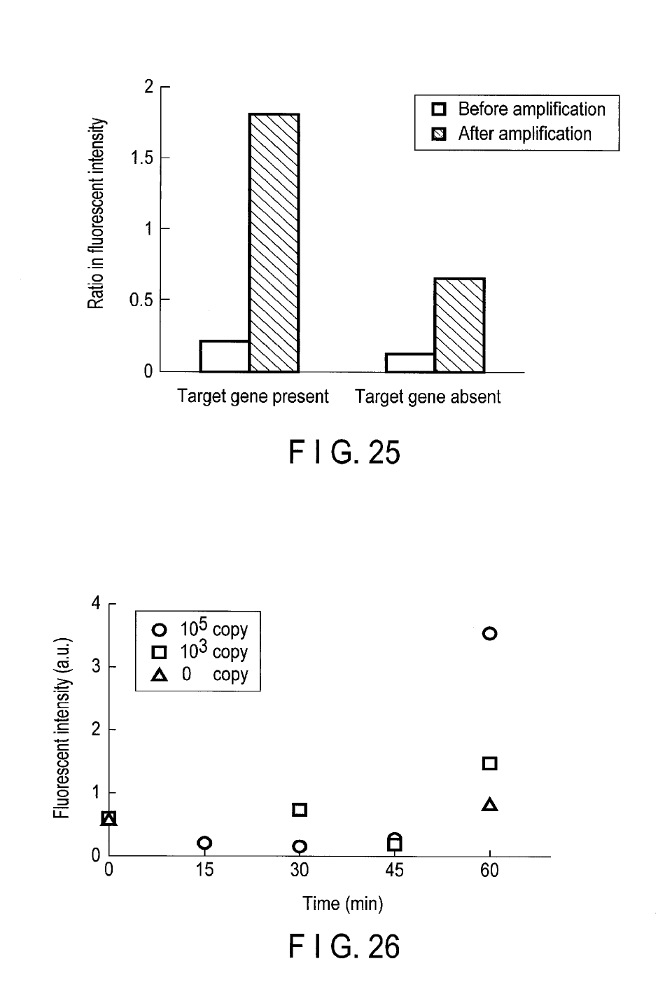

The LAMP amplification was executed on a chip on which the double-stranded nucleic acid probe (C) was immobilized, in the same method as explained above, and the fluorescence was measured after sixty minutes. An experiment was executed with a LAMP reaction solution containing no template, as control. The obtained results are shown in FIG. 25. A fluorescence intensity ratio obtained under a condition of containing no template was approximately 0.7 (explained as “no target gene” in the drawing). In contrast, the fluorescence intensity ratio obtained under a condition of containing a template was increased to approximately 1.8 times (explained as “target gene present” in the drawing). It was clarified from these results that presence or absence of the target nucleic acid can be optically determined by measuring the fluorescence intensity of FAM.

(7) LAMP Amplification Using Array-Type Nucleic Acid Probe Immobilized Substrate for Fluorescence Detection and Detection of Target Nucleic Acid Using Nucleic Acid Probe

Samples having template concentrations of 10⁵ copy/μL and 10³ copy/μL, respectively, were prepared. In contrast, an array-type nucleic acid probe immobilized substrate similar to (6) was prepared. The fluorescence intensities were measured on the array-type nucleic acid probe immobilized substrate for fluorescence detection over time after the start of the amplification reaction. The obtained results are shown in FIG. 26. In the graph, a horizontal axis represents a time (min.) elapsing after the start of the reaction.

As clarified from FIG. 26, increase in the fluorescence intensities depending on concentration of the template nucleic acid was observed at the sixtieth minute after the reaction. In particular, a remarkably strong fluorescence intensity was obtained at a sample having a high template concentration of 10⁵ copy/μL. It was clarified from this result that the target nucleic acid can be optically quantified by monitoring the fluorescence intensity of FAM, etc. from the fluorescent substance.

It is certified based on the above matters that the present embodiment can provide a substrate, a kit and a method capable of measuring a nucleic acid simply at high sensitivity.

### Example 3

Hereinafter, an example of quantifying a target nucleic acid by the method for detecting the nucleic acid according to the third embodiment is described. The labeling substance is contained in the reaction solution. When the target nucleic acid is present in the reaction field, the covering nucleic acid chain is desorbed from the nucleic acid probe, such that the labeling substance is detectable by the corresponding electrode.

Any array-type nucleic acid probe immobilized substrate includes a primer set immobilized to a primer-immobilization region and a probe DNA used as a nucleic acid probe immobilized to a probe immobilization region in close vicinity to the primer-immobilization region. The probe immobilization region was arranged on an electrode and used as a sensor for detecting a current response generated depending on the existence of hybridization.

The covering nucleic acid chains are bonded to the used nucleic acid probe to form two chains. The labeling substance was also present in the reaction solution.

(1) Preparation of Chip Material

A chip was manufactured in the same manner as in Example 1.

(2) Preparation of Array-Type Nucleic Acid Probe Immobilized Substrate

First, a sequence (E) was prepared as a nucleic acid chain included in the nucleic acid probe. A 3′ end of this sequence was labeled with thiol. A nucleic acid chain composed of the sequence (G) was prepared as a covering nucleic acid chain, and hybridized to a nucleic acid probe including the sequence (E) to prepare a two-chain nucleic acid probe (EG). Similarly, a sequence (F) was prepared as a nucleic acid chain included in the nucleic acid probe. A 3′ end of this sequence (F) was labeled with thiol. A one chain nucleic acid probe (F) was prepared without bonding the covering nucleic acid chain. The two-chain nucleic acid probe is possible to be prepared by adding the covering nucleic acid chain on a substrate to which the one chain nucleic acid probe is immobilized.

The two-chain nucleic acid probe and the one chain nucleic acid probe were immobilized on two working electrodes, respectively. Base sequences of the used nucleic acid chains are shown in Table 8.

TABLE 8

| | SEQ | | |
|---|---|---|---|
| Se- | ID | | |
| quence | NO: | | Sequence |
| (E) | 10 | Nucleic | GAAGGCATCCTAAGAAATCGCTACTAC |
| | | acid | |
| | | probes | |
| | | (for two | |
| | | chains) | |
| (F) | 11 | Nucleic | TGCTCGGCTGTATCATGAAACAAAAGGA |
| | | acid | |
| | | probes | |
| | | (for one | |
| | | chain) | |
| (G) | 12 | Coating | GTAGTAGCGATTTCTTAGGATGCCTTCTG |
| | | nucleic | GTATATATTTGTTGGTGTGCCA |
| | | acid of | |
| | | Example 3 | |
| (H) | 13 | Coating | GTAGTAGCGATTTCTTAGGATGCCTTCTGG |
| | | nucleic | TATATATTTGTTGGTGTGCCACTAGTT |
| | | acid of | |
| | | Example 4 | |

Immobilization of these nucleic acid probes was performed by the same method as the immobilization of Example 1, i.e., by immobilizing the nucleic acid probes to two working electrodes of the chip material.

(3) Detection of Electrochemical Signal

The electrochemical response from the 1 mM ferricyanide ion by the two-chain nucleic acid probe and the one chain nucleic acid probe was measured using an electrochemical analyzer ALS660a. The measurement was performed by using cyclic voltammetry, and a potential sweep rate was 0.25 V/s.

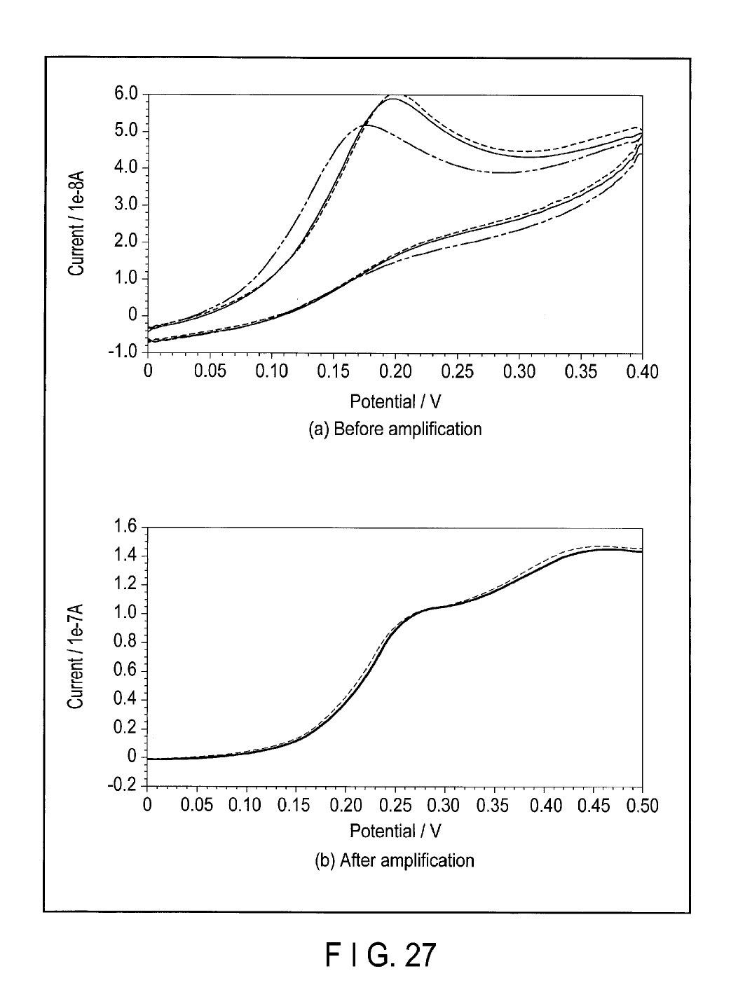

Measurement results are shown in FIGS. 27(a) and (b). Horizontal axes of these graphs represent a potential (V), and vertical axes represent a current (nA).

FIG. 27(a) shows a result of detection from an electrode to which the two-chain nucleic acid probe and the one chain nucleic acid probe are immobilized. It could be appreciated that in a case where the nucleic acid probe had one chain without hybridization with the covering nucleic acid chain, the redox potential obtained from the ferricyanide ion was shifted to positive as compared to a case where the nucleic acid probe has two-chains according to the hybridization between the nucleic acid probe and the covering nucleic acid chain.

(4) Preparation of Primer Set

Next, a primer DNA to be used as a primer set was prepared. The primer DNA to be used is a primer set for amplification by the loop-mediated isothermal amplification (LAMP) method. Base sequences of the primer DNA used are shown in Table 9.

TABLE 9

| SEQ | | |
|---|---|---|
| ID | | |
| NO: | | Sequence |
| 3 | F3 | GAGATATTATTTTCAATGGGATAGAAC |
| 4 | B3 | CAATGCTCTATTTGTTTGCCATG |
| 5 | FIP | GAACATCATCTGGATCTGTACCAACCATCTCATACTGGAACTA |
| | | GTGGC |
| 6 | BIP | CTGTGCCAGTACACTTACTAAGAGTGTTAGTCTACATGGTTTA |
| | | CAATC |
| 8 | LPB | ACAGGTGATGAATTTGCTACAGG |

As for concentrations of primers used for the LAMP amplification reaction, the concentration of F3 primer and B3 primer was 0.12 μM, the concentration of FIP primer and BIP primer was 1.6 μM, and the concentration of LPF primer was 0.4 μM.

(5) Preparation of LAMP Reaction Solution

A composition of a LAMP reaction solution is presented below in Table 10.

TABLE 10

| Components | Concentration | |
|---|---|---|
| Tris-HCl (pH 8.0) | 20 | mM |
| KCl | 10 | mM |
| MgSO4 | 8 | mM |
| (NH4)2SO4 | 10 | mM |
| Tween20 | 0.1% | |
| dNTPs | 1.4 | mM each |
| Bst polymerase | 8 | unit |

0.8 M betaine was added in a general LAMP reaction, but was not used since betaine inhibits a reaction in an electrochemical measurement. 10⁵ copy/μL plasmid (length: approximately 4 kbp) was used as the target nucleic acid used as the template, and the LAMP amplification reaction was performed at 63° C. The plasmid was obtained by inserting a Parvo virus-derived VP gene represented by SEQ ID NO: 9 shown in Table 11 (VP gene of Parvo virus, with a length of 1000 bp) into a pMA vector.

TABLE 11
Parvo virus-derived VP gene (SEQ ID NO: 9)

AAACGCTAATACGACTCACTATAGGGCGATCTACGGGTACTTTCA
ATAATCAGACGGAATTTAAATTTTTGGAAAACGGATGGGIGGAAA
TCACAGCAAACTCAAGCAGACTTGTACATTTAAATATGCCAGAAA
GTGAAAATTATAGAAGAGTGGTTGTAAATAATTTGGATAAAACTG
CAGTTAACGGAAACATGGCTTTAGATGATACTCATGCACAAATTG
TAACACCTTGGTCATTGGTTGATGCAAATGCTTGGGGAGTTTGGT
TTAATCCAGGAGATTGGCAACTAATTGTTAATACTATGAGTGAGT
TGCATTTAGTTAGTTTTGAACAAGAAATTTTTAATGTTGTTTTAA
AGACTGTTTCAGAATCTGCTACTCAGCCACCAACTAAAGTTTATA
ATAATGATTTAACTGCATCATTGATGGTTGCATTAGATAGTAATA
ATACTATGCCATTTACTCCAGCAGCTATGAGATCTGAGACATTGG
GTTTTTATCCATGGAAACCAACCATACCAACTCCATGGAGATATT
ATTUTCAATGOCATAGAACATTAATACCATCTCATACTGGAACTA
GTGGCACACCAACAAATATATACCATGGTACAGATCCAGATGATG
TTCAATTTTATACTATTGAAAATTCTGTGCCAGTACACTTACTAA
GAACAGGTGATGAATTTCCTACAGGAACATTTTTTTTTGATTGTA
AACCATCTAGACTAACACATACATGGCAAACAAATAGAGCATTGG
GCTTACCACCATTTCTAAATTCTTTGCCTCAAGCTGAAGGAGGTA
CTAACTTTGGTTATATAGGAGTTCAACAAGATAAAAGACGTGGTG
TAACTCAAATGGGAAATACAAACTATATTACTGAAGCTACTATTA
TGAGACCAGCTGAGGTTGGTTATAGTGCACCATATTATTCTTTTG
AGGCGTCTACACAAGGGCCATTTAAAACACCCTTCCCTTTAGTGA
GGGTTAATAA

Potassium ferricyanide was added to the LAMP reaction solution to have a concentration of 1 mM.

(6) LAMP Amplification Using Array-Type Nucleic Acid Probe Immobilized Substrate for Electrochemical Detection, and Detection of Target Nucleic Acid by Nucleic Acid Probe

The redox potential of the ferricyanide ion was measured while LAMP amplification was performed on the chip to which the nucleic acid probe was immobilized. Results thereof are shown in FIG. 27(b). Before the amplification, in the one chain nucleic acid probe (F) and the two-chain nucleic acid probe (EG), the redox potential of the ferricyanide ion was different. However, after the amplification, the same waveform of the redox potential was shown.

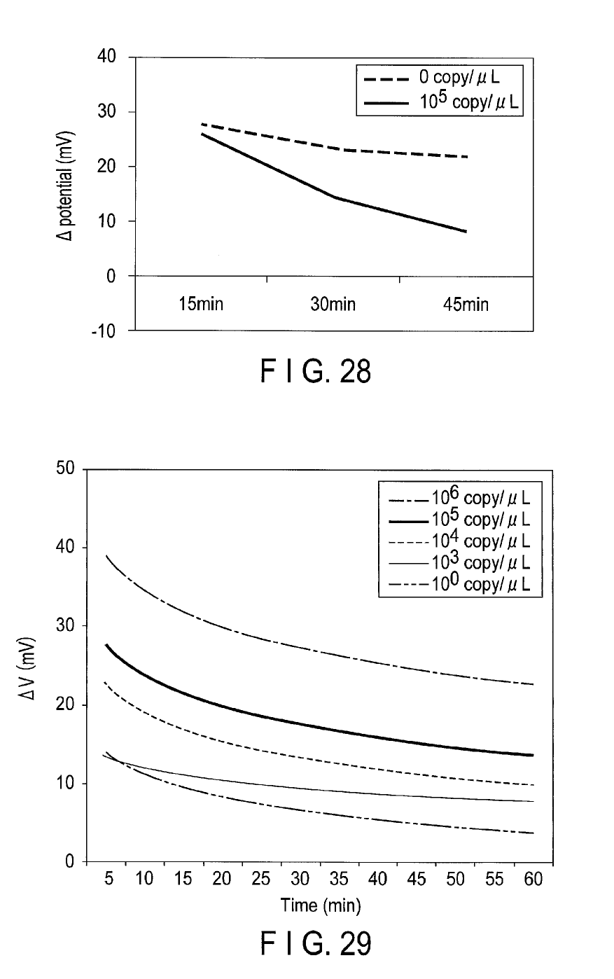

Next, while the LAMP amplification was performed on the chip to which the nucleic acid probe was immobilized, a change over time of an electric signal was measured by allowing the target nucleic acids with 0 copy/μL or 10⁻⁵ copy/μL to exist in reaction fields and monitoring respective electric signals. Results thereof are shown in FIG. 28. A graph of FIG. 28 shows plotting of Δ peak potential which is a potential difference between a control experimental group obtained for the one chain nucleic acid probe (F) and an experimental group obtained for the two-chain nucleic acid probe (EG) with regard to the above concentrations in two-levels. As a result, Δ potential value was decreased over time in both cases where the concentration of the target nucleic acid was 0 copy/μL and where the concentration of the target nucleic acid was 10⁵ copy/μL. However, a slope of the graph showing a decrease rate of Δ potential was larger in the case of 10⁵ copy/μL. Accordingly, it was found from these results that as the concentration of the target nucleic acid present in the reaction solution was lower, the decrease rate of the Δ potential value was slower, and as the concentration of the target nucleic acid was higher, the decrease rate of the Δ potential value was faster. From this, the concentration of the target nucleic acid is capable of being determined, for example, by measuring time required for reaching a specific Δ potential value. Alternatively, the concentration of the target nucleic acid is capable of being determined, for example, by measuring a magnitude of the Δ potential at a specific time. That is, the specific Δ potential value and the specific time may be set as threshold values. From the above, it has been found that the target nucleic acid is capable of being quantitatively detected by an electrochemical method in which the potential of the ferricyanide ion is monitored and the time at which the Δ peak potential is changed is monitored.

From the above, it has been proved that it is possible to provide a substrate, a kit, and a method in which a nucleic acid is capable of being measured simply at high sensitivity according to the present embodiment.

### Example 4

Hereinafter, an example of quantifying a target nucleic acid by the method for detecting the nucleic acid according to the fourth embodiment is described. This is an example as follows. An isothermal amplification reaction of the target nucleic acid is performed in a state in which a labeling substance is included in a reaction solution. When the amplification product is formed by the reaction, the amplification product is bonded to the covering nucleic acid chain in a state where the covering nucleic acid chain is bonded to the nucleic acid probe. Then, the covering nucleic acid chain is elongated using the amplification product bonded thereto as a template. Accordingly, the labeling substance is distant from the electrode than that of the initial reaction.

An example of a method for detecting a target nucleic acid for detecting a signal by elongation of a covering nucleic acid chain from a labeling substance is described.

Any array-type nucleic acid probe immobilized substrate was used.

The same sensor as Example 3 was used.

(1) Preparation of Chip Material

A chip was manufactured in the same manner as in Example 1.

(2) Preparation of Array-Type Nucleic Acid Probe Immobilized Substrate

First, a sequence (E) was prepared as a nucleic acid chain included in the nucleic acid probe. A 3′ end of this sequence (E) was labeled with thiol. A nucleic acid chain composed of the sequence (H) was prepared as a covering nucleic acid chain, and hybridized to a nucleic acid probe including the sequence (E) to prepare a two-chain nucleic acid probe (EH). Similarly, a sequence (F) was prepared as a nucleic acid chain included in the nucleic acid probe. A 3′ end of this sequence (F) was labeled with thiol. A one chain nucleic acid probe (F) was prepared without bonding the covering nucleic acid chain. The two-chain nucleic acid probe is possible to be prepared by adding the covering nucleic acid chain on a substrate to which the one chain nucleic acid probe is immobilized.

The two-chain nucleic acid probe (E) and the one chain nucleic acid probe (F) were immobilized on two working electrodes, respectively. Base sequences of the used nucleic acid chains are shown in Table 8.

Immobilization of these nucleic acid probes was performed by the same method as the immobilization of Example 1, i.e., by immobilizing the nucleic acid probes to two working electrodes of the chip material.

(3) Detection of Electrochemical Signal

The electrochemical signal was detected by the same method as Example 3.

(4) Preparation of Primer Set

The same primer set as Example 3 was prepared, and used at the same concentration as Example 3.

(5) Preparation of LAMP Reaction Solution

A composition of the LAMP reaction solution is shown in Table 12 below. GspSSD was used as an enzyme and a KCl concentration was adjusted to 60 mM. The same target nucleic acid and the same labeling substance as Example 3 were used.

TABLE 12

| Components | Concentration | |
|---|---|---|
| Tris-HCl (pH 8.0) | 20 | mM |
| KCl | 60 | mM |
| MgSO4 | 8 | mM |
| (NH4)2SO4 | 10 | mM |
| Tween20 | 0.1% | |
| dNTPs | 1.4 | mM each |
| GspSSD polymerase | 8 | unit |

(6) LAMP Amplification Using Array-Type Nucleic Acid Probe Immobilized Substrate for Electrochemical Detection, and Detection of Target Nucleic Acid by Nucleic Acid Probe

The LAMP amplification was performed on the chip to which the nucleic acid probe was immobilized, and a change over time of an electric signal was measured by allowing the target nucleic acids with 0¹, 10³, 10⁴, 10⁵, and 10⁶ copy/μL to exist in reaction fields and monitoring respective electric signals. Accordingly, a redox potential of ferricyanide ions was measured. Results thereof are shown in FIG. 29. A graph of FIG. 29 shows plotting of Δ peak potential which is a potential difference between a control experimental group obtained for the one chain nucleic acid probe (F) and an experimental group obtained for the two-chain nucleic acid probe (EH) with regard to the above concentrations in five levels. As a result, Δ potential value was decreased over time in all concentrations of the target nucleic acid. A decrease rate of potential was smaller as the concentrations of the target nucleic acid were higher. Accordingly, it was found from these results that as the concentration of the target nucleic acid present in the reaction solution was lower, the decrease rate of the potential value was faster, and as the concentration of the target nucleic acid was higher, the decrease rate of the Δ potential value was slower. From this, the concentration of the target nucleic acid is capable of being determined, for example, by measuring time required for reaching a specific Δ potential value. Alternatively, the concentration of the target nucleic acid is capable of being determined, for example, by measuring a magnitude of the Δ potential at a specific time. That is, the specific Δ potential value and the specific time may be set as threshold values. From the above, it has been found that the target nucleic acid is capable of being quantitatively detected by an electrochemical method in which the potential of the ferricyanide ion is monitored and the time at which the Δ peak potential is changed is monitored.

As set forth above, it has been proved that it is possible to provide a substrate, a kit, and a method in which a nucleic acid is capable of being measured simply at high sensitivity according to the present embodiment. It has been proved that it is possible to provide a substrate, a kit, and a method in which a redox potential is different depending on concentrations of a template, and a nucleic acid is capable of being measured simply at high sensitivity, according to the present embodiment.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A method for detecting a target nucleic acid,
the target nucleic acid including a first sequence and/or a complementary sequence thereof,
the method comprising:
(A) placing a reaction field formed by a reaction solution under an isothermal amplification reaction condition,
the reaction solution including
a sample which includes the target nucleic acid,
a nucleic acid probe immobilized to at least one surface of a substrate, the nucleic acid probe including a second sequence different from the first sequence,
a covering nucleic acid chain which includes a first sequence bonding region complementary to the first sequence and a second sequence bonding region complementary to the second sequence, the covering nucleic acid chain being bonded to the nucleic acid probe via hybridization to the second sequence in the second sequence bonding region,
a labeling substance which produces a detectable signal, and
a primer set for forming an amplification product including the first sequence,
(B) monitoring the signal from the nucleic acid probe or detecting the signal at two or more time points, under the isothermal amplification reaction condition; and
(C) obtaining a detection result for the target nucleic acid based on the signal for the sample obtained in (B),
wherein each of a sequence of the nucleic acid probe and a sequence of the covering nucleic acid chain is
(a) a sequence for obtaining competition of the amplification product and the nucleic acid probe to the covering nucleic acid chain, desorption of the covering nucleic acid chain from the nucleic acid probe by the competition, and bonding via hybridization between the first sequence bonding region of the covering nucleic acid chain and the first sequence of the amplification product, under the isothermal amplification reaction condition, or
(b) a sequence for obtaining bonding via hybridization between the first sequence bonding region of the covering nucleic acid chain and the first sequence of the amplification product, and elongation of the covering nucleic acid chain using the amplification product as a template, with the bonding of the nucleic acid probe and the covering nucleic acid chain being maintained, under the isothermal amplification reaction condition, and
the detection of the detectable signal produced by the labeling substance is inhibited by a presence of the nucleic acid which is bonded to the nucleic acid probe.

2. The method of claim 1, wherein
each of the sequence of the nucleic acid probe and the covering nucleic acid chain are the sequences of (a), and
lengths and Tm values of the base sequence of the nucleic acid probe and the covering nucleic acid chain are set such that, under the isothermal amplification reaction condition, the bonding via hybridization between the nucleic acid probe and the covering nucleic acid chain is maintained if the target nucleic acid is not present in the reaction field, and that the bonding is eliminated if the target nucleic acid is present in the reaction field and if the target nucleic acid and the nucleic acid probe are competitive to the covering nucleic acid chain.

3. The method of claim 1, wherein
each of the sequence of the nucleic acid probe and the covering nucleic acid chain are the sequences of (b), and
lengths and Tm values of the base sequence of the nucleic acid probe and the covering nucleic acid chain are set such that, under the isothermal amplification reaction condition, the bonding via hybridization between the nucleic acid probe and the covering nucleic acid chain is maintained regardless of both cases where the target nucleic acid is present in the reaction field, and where the target nucleic acid is not present in the reaction field.

4. The method of claim 1, wherein
the condition of the isothermal amplification reaction includes a temperature condition of the reaction field in a range from 25 to 70° C.

5. The method of claim 1, wherein
the condition of the isothermal amplification reaction includes a salt concentration condition of the reaction field in a range from 10 to 120 mM.

6. The method of claim 1, wherein
a base length of the covering nucleic acid chain is longer than a base length of the nucleic acid probe.

7. The method of claim 1, wherein
the first sequence bonding region and the second sequence bonding region are arranged independently on the covering nucleic acid chain or mutually superposed partially or entirely on the covering nucleic acid chain.

8. The method of claim 1, wherein
the labeling substance is an electrochemically active substance or an optically active substance.

9. The method of claim 1, wherein
the labeling substance bind to the nucleic acid chain of the nucleic acid probe, and is an electrochemically active substance selected from a group consisting of anthraquinone, ferrocene, and methylene blue or an optically active substance selected from a group consisting of Alexa flour, BODIPY, Cy3, Cy5, FAM, Fluorescein, HEX, JOE, Marina Blue (trademark), Oregon Green, Pacific Blue (trademark), Rhodamine, Rhodol Green, ROX, TEMRA, TET and Texas Red (registered trademark).

10. The method of claim 1, wherein
the labeling substance is an electrochemically active substance which is dispersed in the reaction solution and is selected from the group consisting of ferricyanide ions, ferrocyanide ions, iron complex ions, ruthenium complex ions, and cobalt complex ions.

11. The method of claim 1, wherein
the primer set is releasably immobilized to the surface of the substrate to which the nucleic acid probe is immobilized before the reaction field is formed, and
bringing of the primer set into the reaction field is achieved by release from the substrate to the reaction solution.

12. The method of claim 1, which comprises:
(D) placing the reaction field comprising a control probe and the labeling substance which produces the detectable signal under the same condition as the isothermal amplification reaction condition described in (A),
(E) monitoring the signal from the nucleic acid probe or detecting the signal at two or more time points, under the isothermal amplification reaction condition, and
(F) obtaining a detection result for the target nucleic acid by comparing the signal for the sample obtained in (B) and the signal from the control probe obtained in (E), while simultaneously performing (A) and (B) on the sample.

13. The method of claim 1, wherein detection of the target nucleic acid is quantitative detection.

14. An assay kit for detecting a target nucleic acid,
the assay kit comprising:
a primer set for amplifying the target nucleic acid,
a probe immobilized substrate for performing an isothermal amplification reaction and detecting an amplification product obtained by the isothermal amplification reaction,
a labeling substance which produces a detectable electrochemical signal and,
the target nucleic acid including a first sequence and/or a complementary sequence thereof,
the primer set including a primer for amplifying the first sequence and/or the complementary sequence,
the probe immobilized substrate including:
a substrate configured to support a reaction field in which the isothermal amplification reaction is performed;
a probe immobilization region arranged on at least one surface of the substrate in contact with the reaction field when the reaction field is formed;
a nucleic acid probe including a second sequence immobilized to the probe immobilization region; and
a covering nucleic acid chain which includes a first sequence bonding region complementary to the first sequence and a second sequence bonding region complementary to the second sequence, the covering nucleic acid chain being bonded to the nucleic acid probe via hybridization to the second sequence in the second sequence bonding region,
wherein each of a sequence of the nucleic acid probe and a sequence of the covering nucleic acid chain is
(a) a sequence for obtaining competition of the amplification product and the nucleic acid probe to the covering nucleic acid chain, desorption of the covering nucleic acid chain from the nucleic acid probe by the competition, and bonding via hybridization between the first sequence bonding region of the covering nucleic acid chain and the first sequence of the amplification product, under the isothermal amplification reaction condition in the formed reaction field, or
(b) a sequence for obtaining the bonding via hybridization between the first sequence bonding region of the covering nucleic acid chain and the first sequence of the amplification product, and elongation of the covering nucleic acid chain using the amplification product as a template, with the bonding between the nucleic acid probe and the covering nucleic acid chain being maintained, under the isothermal amplification reaction condition in the formed reaction field,
the detection of the detectable signal produced by the labeling substance is inhibited by a presence of the nucleic acid which is bonded to the nucleic acid probe, and
the labeling substance being independent from the probe immobilized substrate, or being indirectly immobilized or being releasably and directly immobilized at a position corresponding to the nucleic acid probe on the at least one surface.

15. The assay kit of claim 14, wherein
each of the sequence of the nucleic acid probe and the covering nucleic acid chain are the sequences of (a), and
lengths and Tm values of the base sequence of the nucleic acid probe and the covering nucleic acid chain are set such that, under the isothermal amplification reaction condition, the bonding via hybridization between the nucleic acid probe and the covering nucleic acid chain is maintained if the target nucleic acid is not present in the reaction field, and that the bonding is eliminated if the target nucleic acid is present in the reaction field and if the target nucleic acid and the nucleic acid probe are competitive to the covering nucleic acid chain.

16. The assay kit of claim 14, wherein
each of the sequence of the nucleic acid probe and the covering nucleic acid chain are the sequences of (b),
the labeling substance is dispersed in the reaction solution, and
lengths and Tm values of the base sequence of the nucleic acid probe and the covering nucleic acid chain are set such that, under the isothermal amplification reaction condition, the bonding via hybridization between the nucleic acid probe and the covering nucleic acid chain is maintained regardless of both cases where the target nucleic acid is present in the reaction field, and where the target nucleic acid is not present in the reaction field.

17. The assay kit of claim 14, wherein
the labeling substance is an electrochemically active substance or an optically active substance.

18. The assay kit of claim 14, wherein
the labeling substance is bonded to the nucleic acid chain of the nucleic acid probe, and is an electrochemically active substance selected from the group consisting of anthraquinone, ferrocene, and methylene blue, or an optically active substance selected from the group consisting of Alexa flour, BODIPY, Cy3, Cy5, FAM, Fluorescein, HEX, JOE, Marina Blue (trademark), Oregon Green, Pacific Blue (trademark), Rhodamine, Rhodol Green, ROX, TEMRA, TET and Texas Red (registered trademark).

19. The assay kit of claim 14, wherein
the labeling substance is an electrochemically active substance which is dispersed in the reaction solution and is selected from the group consisting of ferricyanide ions, ferrocyanide ions, iron complex ions, ruthenium complex ions, and cobalt complex ions.

20. The assay kit of claim 14, wherein
the primer set is releasably immobilized to the primer-immobilized region arranged on at least one surface of the substrate in contact with the reaction field when the reaction field is formed.

21. The assay kit of claim 14, further comprising:
at least one control probe immobilization region arranged independently on at least one surface of the substrate in contact with the reaction field when the reaction field is formed, and
a positive control probe and/or a negative control probe immobilized to the control probe immobilization region.

22. The assay kit of claim 14, wherein
the target nucleic acid is first to n-th target nucleic acids, and the first to n-th target nucleic acids include 1₁-th to 1ₙ-th sequences and/or first to n-th complementary sequences thereof, respectively,
the primer set includes a plurality of primer groups respectively including primers configured to amplify the 1₁-th to 1ₙ-th sequences, respectively, and
in the probe immobilized substrate,
the substrate is configured to support a reaction field in which an isothermal amplification reaction using first to n-th primer sets produces first to n-th amplification products including the 1₁-th to mutually different 1ₙ-th sequences, respectively, using the first to n-th target nucleic acids as templates, respectively,
first to n-th probe immobilization regions arranged independently on at least one surface of the substrate in contact with the reaction field when the reaction field is formed, are included as the probe immobilization region,
a nucleic acid probe group respectively including first to n-th nucleic acid chains respectively including 2₁-th to 2ₙ-th sequences immobilized respectively to the respective first to n-th probe immobilization regions, is included as the nucleic acid probe, and
first to n-th covering nucleic acid chains which respectively include 1₁-th to 1ₙ-th sequence bonding regions that are respectively complementary to the respective 1₁-th to 1ₙ-th sequences, and 2₁-th to 2ₙ-th sequence bonding regions that are respectively complementary to the respective 2₁-th to 2ₙ-th sequences, the first to n-th covering nucleic acid chains being bonded to respective first to n-th nucleic acid probes via hybridization with the respective 2₁-th to 2ₙ-th sequences in the respective 2₁-th to 2ₙ-th sequence bonding regions, are included as the covering nucleic acid chain, and
wherein each of a sequence of the first to n-th nucleic acid probes and the first to n-th covering nucleic acid chains are
(a) a sequence for obtaining respective competitions of the first to n-th amplification products and the first to n-th nucleic acid probe sequences corresponding to the respective first to n-th nucleic acid chains, desorption of the first to n-th covering nucleic acid chains from the respective first to n-th nucleic acid probes by the competitions, and respective bondings via respective hybridizations between the 1₁-th to 1ₙ-th sequence bonding regions of the first to n-th covering nucleic acid chains and the 1₁-th to 1ₙ-th sequences of the first to n-th amplification products, under an isothermal amplification reaction condition in the formed reaction field, or
(b) a sequence for obtaining respective bondings via respective hybridizations between the 1₁-th to 1ₙ-th sequence bonding regions of the respective first to n-th covering nucleic acid chains and the 1₁-th to 1ₙ-th sequences of the first to n-th amplification products, and respective elongations of the first to n-th covering nucleic acid chains using the respective first to n-th amplification products as templates, under the isothermal amplification reaction condition in the formed reaction field.

23. A probe immobilized substrate for detecting a target nucleic acid,
the target nucleic acid including a first sequence and/or a complementary sequence thereof,
the probe immobilized substrate comprising:
a substrate configured to support a reaction field in which an isothermal amplification reaction for amplifying the first sequence and/or the complementary sequence thereof using a primer set to obtain an amplification product is performed;
a probe immobilization region arranged on at least one surface of the substrate in contact with the reaction field when the reaction field is formed,
a nucleic acid probe including a second sequence immobilized to the probe immobilization region,
a covering nucleic acid chain which includes a first sequence bonding region complementary to the first sequence and a second sequence bonding region complementary to the second sequence, the covering nucleic acid chain being bonded to the nucleic acid probe via hybridization to the second sequence in the second sequence bonding region, and
a labeling substance which produces a detectable signal that is indirectly immobilized or releasably and directly immobilized at a position corresponding to the nucleic acid probe on the at least one surface,
wherein each of a sequence of the nucleic acid probe and a sequence of the covering nucleic acid chain is
(a) a sequence for obtaining competition of the amplification product and the nucleic acid probe to the covering nucleic acid chain, desorption of the covering nucleic acid chain from the nucleic acid probe by the competition, and bonding via hybridization between the first sequence bonding region of the covering nucleic acid chain and the first sequence of the amplification product, under the isothermal amplification reaction condition in the formed reaction field, or
(b) a sequence for obtaining the bonding via hybridization between the first sequence bonding region of the covering nucleic acid chain and the first sequence of the amplification product, and elongation of the covering nucleic acid chain using the amplification product as a template, with the bonding between the nucleic acid probe and the covering nucleic acid chain being maintained, under the isothermal amplification reaction condition in the formed reaction field, and
the detection of the detectable signal produced by the labeling substance is inhibited by a presence of the nucleic acid which is bonded to the nucleic acid probe.

24. The probe immobilized substrate of claim 23, wherein
each of the sequence of the nucleic acid probe and the covering nucleic acid chain are the sequences of (a), and
lengths and Tm values of the base sequence of the nucleic acid probe and the covering nucleic acid chain are set such that, under the isothermal amplification reaction condition in the formed reaction field, the bonding via hybridization between the nucleic acid probe and the covering nucleic acid chain is maintained if the target nucleic acid is not present in the reaction field, and that the bonding is eliminated if the target nucleic acid is present in the reaction field and if the target nucleic acid and the nucleic acid probe are competitive to the covering nucleic acid chain.

25. The probe immobilized substrate of claim 23, wherein
each of the sequence of the nucleic acid probe and the covering nucleic acid chain are the sequences of (b),
lengths and Tm values of the base sequence of the nucleic acid probe and the covering nucleic acid chain are set such that, under the isothermal amplification reaction condition in the formed reaction field, the bonding via hybridization between the nucleic acid probe and the covering nucleic acid chain is maintained regardless of both cases where the target nucleic acid is present in the reaction field, and where the target nucleic acid is not present in the reaction field.

26. The probe immobilized substrate of claim 23, wherein
the labeling substance is an electrochemically active substance or an optically active substance.

27. The probe immobilized substrate of claim 23, wherein
the labeling substance is bonded to the nucleic acid chain of the nucleic acid probe, and is an electrochemically active substance selected from the group consisting of anthraquinone, ferrocene, and methylene blue, or an optically active substance selected from the group consisting of Alexa flour, BODIPY, Cy3, Cy5, FAM, Fluorescein, HEX, JOE, Marina Blue (trademark), Oregon Green, Pacific Blue (trademark), Rhodamine, Rhodol Green, ROX, TEMRA, TET and Texas Red (registered trademark).

28. The probe immobilized substrate of claim 23, wherein
the labeling substance is an electrochemically active substance which is dispersed in the reaction solution and is selected from the group consisting of ferricyanide ions, ferrocyanide ions, iron complex ions, ruthenium complex ions, and cobalt complex ions.

29. The probe immobilized substrate of claim 23, wherein
the primer set is releasably immobilized to a primer-immobilization region arranged on at least one surface of the substrate in contact with the reaction field when the reaction field is formed.

30. The probe immobilized substrate of claim 23, which comprises:
at least one control probe immobilization region arranged independently on at least one surface of the substrate in contact with the reaction field when the reaction field is formed, and
a positive control probe and/or a negative control probe immobilized to the control probe immobilization region.

31. The probe immobilized substrate of claim 23, wherein
the target nucleic acid is first to n-th target nucleic acids, and the first to n-th target nucleic acids include 1₁-th to 1ₙ-th sequences and/or first to n-th complementary sequences thereof, respectively,
the primer set includes a plurality of primer groups respectively including primers configured to amplify the 1₁-th to 1ₙ-th sequences, respectively, and
in the probe immobilized substrate,
the substrate is configured to support a reaction field in which an isothermal amplification reaction using first to n-th primer sets produces first to n-th amplification products including the 1₁-th to mutually different 1ₙ-th sequences, respectively, using the first to n-th target nucleic acids as templates, respectively,
first to n-th probe immobilization regions arranged independently on at least one surface of the substrate in contact with the reaction field when the reaction field is formed, are included as the probe immobilization region,
a nucleic acid probe group respectively including first to n-th nucleic acid chains respectively including 2₁-th to 2ₙ-th sequences immobilized respectively to the respective first to n-th probe immobilization regions, is included as the nucleic acid probe, and
first to n-th covering nucleic acid chains which respectively include 1₁-th to 1ₙ-th sequence bonding regions that are respectively complementary to the respective 1₁-th to 1ₙ-th sequences, and 2₁-th to 2ₙ-th sequence bonding regions that are respectively complementary to the respective 2₁-th to 2ₙ-th sequences, the first to n-th covering nucleic acid chains being bonded to respective first to n-th nucleic acid probes via hybridization with the respective 2₁-th to 2ₙ-th sequences in the respective 2₁-th to 2ₙ-th sequence bonding regions, are included as the covering nucleic acid chain, and
wherein each of a sequence of the first to n-th nucleic acid probes and the first to n-th covering nucleic acid chains are
(a) a sequence for obtaining respective competitions of the first to n-th amplification products and the first to n-th nucleic acid probe sequences corresponding to the respective first to n-th nucleic acid chains, desorption of the first to n-th covering nucleic acid chains from the respective first to n-th nucleic acid probes by the competitions, and respective bondings via respective hybridizations between the 1₁-th to 1ₙ-th sequence bonding regions of the first to n-th covering nucleic acid chains and the 1₁-th to 1ₙ-th sequences of the first to n-th amplification products, under an isothermal amplification reaction condition in the formed reaction field, or
(b) a sequence for obtaining respective bondings via respective hybridizations between the 1₁-th to 1ₙ-th sequence bonding regions of the respective first to n-th covering nucleic acid chains and the 1₁-th to 1ₙ-th sequences of the first to n-th amplification products, and respective elongations of the first to n-th covering nucleic acid chains using the respective first to n-th amplification products as templates, under the isothermal amplification reaction condition in the formed reaction field.
